SEQUENCE LISTING (46 sequences)

SEQ ID NO: 1 - DNA, 28 bp, Artificial Sequence
  Description of Artificial Sequence Synthetic primer
tctagactaatgaaaccctgatatcaac

SEQ ID NO: 2 - DNA, 29 bp, Artificial Sequence
  Description of Artificial Sequence Synthetic primer
tctagaactgctaccctgaggcacagcct

SEQ ID NO: 3 - DNA, 30 bp, Artificial Sequence
  Description of Artificial Sequence Synthetic primer
tctagactggatgggactacctttctgttc

SEQ ID NO: 4 - DNA, 29 bp, Artificial Sequence
  Description of Artificial Sequence Synthetic primer
tctagacataatcctctgagaataggccg

SEQ ID NO: 5 - DNA, 34 bp, Artificial Sequence
  Description of Artificial Sequence Synthetic primer
tctagagtgacatgcaatggcaactcatgtggac

SEQ ID NO: 6 - DNA, 34 bp, Artificial Sequence
  Description of Artificial Sequence Synthetic primer
tctagaacaaagaatccccaacacacccccccat

SEQ ID NO: 7 - DNA, 32 bp, Artificial Sequence
  Description of Artificial Sequence Synthetic primer
tctagatgatgccctgagagcccactgcagtt

SEQ ID NO: 8 - DNA, 31 bp, Artificial Sequence
  Description of Artificial Sequence Synthetic primer
tctagattgcttcactgccaggagcccctga

SEQ ID NO: 9 - DNA, 40 bp, Artificial Sequence
  Description of Artificial Sequence Synthetic primer
gctctagagcgcagcacaaggccttgcctggcctgatgat

SEQ ID NO: 10 - DNA, 40 bp, Artificial Sequence
  Description of Artificial Sequence Synthetic primer
gctctagagccatggcagtgggtaacacgtcctctttcac

SEQ ID NO: 11 - DNA, 43 bp, Artificial Sequence
  Description of Artificial Sequence Synthetic primer
gctctagatccctgtgtgtgtgtatgtgtgtgcatgtgtgcgt

SEQ ID NO: 12 - DNA, 40 bp, Artificial Sequence
  Description of Artificial Sequence Synthetic primer
gctctagagcggagagggatttgagagctcgctggggtga

SEQ ID NO: 13 - DNA, 35 bp, Artificial Sequence
  Description of Artificial Sequence Synthetic primer
gtggttggatgaataatattaatctcctttttccc

SEQ ID NO: 14 - DNA, 35 bp, Artificial Sequence
  Description of Artificial Sequence Synthetic primer
gggaaaaaggagattaatattattcatccaaccac

SEQ ID NO: 15 - DNA, 32 bp, Artificial Sequence
  Description of Artificial Sequence Synthetic primer
gtgtaagaatggtgcagtgtgttttccccctc

SEQ ID NO: 16 - DNA, 32 bp, Artificial Sequence
  Description of Artificial Sequence Synthetic primer
gagggggaaaacacactgcaccattcttacac

SEQ ID NO: 17 - DNA, 41 bp, Artificial Sequence
  Description of Artificial Sequence Synthetic primer
gagatttttgtatatagtgttaggcctgtggaattaattcg

SEQ ID NO: 18 - DNA, 41 bp, Artificial Sequence
  Description of Artificial Sequence Synthetic primer
cgaattaattccacaggcctaacactatatacaaaaatctc

SEQ ID NO: 19 - DNA, 42 bp, Artificial Sequence
  Description of Artificial Sequence Synthetic primer
cgttgcatatagaggtatcaatgttcaggcatattataaaac

SEQ ID NO: 20 - DNA, 42 bp, Artificial Sequence
  Description of Artificial Sequence Synthetic primer
gttttataatatgcctgaacattgatacctctatatgcaacg

SEQ ID NO: 21 - DNA, 34 bp, Artificial Sequence
  Description of Artificial Sequence Synthetic primer
ccaaacatgttgtaccatggccccctcatcatag

SEQ ID NO: 22 - DNA, 34 bp, Artificial Sequence
  Description of Artificial Sequence Synthetic primer
ctatgatgagggggccatggtacaacatgtttgg

SEQ ID NO: 23 - DNA, 40 bp, Artificial Sequence
  Description of Artificial Sequence Synthetic primer
ggccaagcagtgcctgcctatgaacttttcctttcatacg

SEQ ID NO: 24 - DNA, 40 bp, Artificial Sequence
  Description of Artificial Sequence Synthetic primer
cgtatgaaaggaaaagttcataggcaggcactgcttggcc

SEQ ID NO: 25 - DNA, 23 bp, Artificial Sequence
  Description of Artificial Sequence Synthetic probe
tcatagccctgtacaatgctgct

SEQ ID NO: 26 - DNA, 22 bp, Artificial Sequence
  Description of Artificial Sequence Synthetic probe
ctagtggtcctaaacatttcac

SEQ ID NO: 27 - DNA, 22 bp, Artificial Sequence
  Description of Artificial Sequence Synthetic probe
agctgagtgtaggatgtttaca

SEQ ID NO: 28 - DNA, 23 bp, Artificial Sequence
  Description of Artificial Sequence Synthetic probe
gctgagagtgtaggatgtttaca

SEQ ID NO: 29 - DNA, 23 bp, Artificial Sequence
  Description of Artificial Sequence Synthetic probe
gaaacccagcagacaatgtagct

SEQ ID NO: 30 - DNA, 24 bp, Artificial Sequence
  Description of Artificial Sequence Synthetic probe
acccagtagccagatgtagtagct

SEQ ID NO: 31 - DNA, 21 bp, Artificial Sequence
  Description of Artificial Sequence Synthetic probe
acccagtagccagatgtagct

SEQ ID NO: 32 - DNA, 23 bp, Artificial Sequence
  Description of Artificial Sequence Synthetic probe
agcagcattgtacagggctatga

SEQ ID NO: 33 - DNA, 22 bp, Artificial Sequence
  Description of Artificial Sequence Synthetic probe
ctagtggtcctaaacatttcac

SEQ ID NO: 34 - DNA, 30 bp, Artificial Sequence
  Description of Artificial Sequence Synthetic primer
ggccggccctaatgaaaccctgatatcaac

SEQ ID NO: 35 - DNA, 31 bp, Artificial Sequence
  Description of Artificial Sequence Synthetic primer
ggccggccactgctaccctgaggcacagcct

SEQ ID NO: 36 - DNA, 32 bp, Artificial Sequence
  Description of Artificial Sequence Synthetic primer
ggccggccctggatgggactacctttctgttc

SEQ ID NO: 37 - DNA, 31 bp, Artificial Sequence
  Description of Artificial Sequence Synthetic primer
ggccggcccataatcctctgagaataggccg

SEQ ID NO: 38 - DNA, 22 bp, Artificial Sequence
  Description of Artificial Sequence Synthetic oligonucleotide
gatgaataatattaatgtagct

SEQ ID NO: 39 - DNA, 23 bp, Artificial Sequence
  Description of Artificial Sequence Synthetic oligonucleotide
agttggactttgccgatgctgca

SEQ ID NO: 40 - DNA, 23 bp, Artificial Sequence
  Description of Artificial Sequence Synthetic oligonucleotide
tttgtatatagtgtttgctgcaa

SEQ ID NO: 41 - DNA, 23 bp, Artificial Sequence
  Description of Artificial Sequence Synthetic oligonucleotide
tatagaggtatcaactgctgcat

SEQ ID NO: 42 - DNA, 21 bp, Artificial Sequence
  Description of Artificial Sequence Synthetic oligonucleotide
tgtgtaagaatggtgtttaca

SEQ ID NO: 43 - DNA, 23 bp, Artificial Sequence
  Description of Artificial Sequence Synthetic oligonucleotide
tcacttccttgcccccatttcac

SEQ ID NO: 44 - DNA, 23 bp, Artificial Sequence
  Description of Artificial Sequence Synthetic oligonucleotide
tactttgtcccgtggcatttcaa

SEQ ID NO: 45 - DNA, 21 bp, Artificial Sequence
  Description of Artificial Sequence Synthetic oligonucleotide
caaacatgttgtacatttcac

SEQ ID NO: 46 - DNA, 23 bp, Artificial Sequence
  Description of Artificial Sequence Synthetic oligonucleotide
gtgcctgcctatgaaatttcaac

(19) United States
(12) Patent Application Publication
(10) Pub. No.: US 2014/0351963 A1
(43) Pub. Date: Nov. 27, 2014

(54) MiRNAs Useful to Reduce Lung Cancer Tumorigenesis and Chemotherapy Resistance and Related Compositions and Methods

(71) Applicant: Ohio State Innovation Foundation, Columbus, OH (US)

(72) Inventors: Carlo M. Croce, Columbus, OH (US); Michela Garofalo, Columbus, OH (US)

(73) Assignee: OHIO STATE INNOVATION FOUNDATION, Columbus, OH (US)

(21) Appl. No.: 14/364,163

(22) PCT Filed: Dec. 10, 2012
(86) PCT No.: PCT/US12/68736

§ 371 Date: Jun. 10, 2014

Related U.S. Application Data
(60) Provisional application No. 61/569,237, filed on Dec. 10, 2011.

(51) Int. Cl. C12N 15/113; A61K 31/5377; A61K 31/496; A61K 45/06

(52) U.S. Cl. CPC C12N 15/1135; A61K 45/06; A61K 31/5377; A61K 31/496; C12N 2310/141; C12N 2320/31
USPC 800/8; 536/24.5; 514/44 A; 435/375

(57) ABSTRACT

Disclosed are compositions, such as nucleic acids, vectors, cells, animal models and the like, useful to reduce tumor growth, cancer cell migration and various other cancer pathologies associated with EGFR (epidermal growth factor receptor) and MET (the receptor tyrosine kinase for hepatocyte growth factors) dyregulation, particularly in non-small cell lung carcinoma.

### CROSS-REFERENCE TO RELATED APPLICATIONS

This application claims the benefit of U.S. Provisional Patent Application No. 61/569,237, filed Dec. 10, 2011, the disclosure of which is incorporated herein by reference for all purposes.

### STATEMENT REGARDING FEDERALLY SPONSORED RESEARCH

This invention was made with government support under Grant No. CA113001 awarded by the National Institutes of Health. The U.S. government has certain rights in the invention.

### BACKGROUND OF THE INVENTION

There is no admission that the background art disclosed in this section legally constitutes prior art.

MiRNAs repress gene expression by inhibiting mRNA translation or by promoting mRNA degradation and are considered to be master regulators of various processes, ranging from proliferation to apoptosis. Both loss and gain of miRNA function contribute to cancer development through the upregulation and silencing, respectively, of different target genes.

Non small cell lung cancers (NSCLCs) account for approximately 85% of all cases of lung cancer Although NSCLC is a remarkably heterogeneous disease that includes distinct morphological and molecular subtypes, activation of epidermal growth factor receptor (EGFR) and MET (the receptor tyrosine kinase (RTK) for hepatocyte growth factors) is common and is associated with stimulation of the rat sarcoma (RAS)-mitogen-activated protein kinase 1 (ERK) and the phosphoinositide-3-kinase (PI3K)-v-akt murine thymoma viral oncogene homolog 1 (AKT) axes, which leads to NSCLC cell proliferation, survival and invasion.

The tyrosine-kinase inhibitors (TKIs) gefitinib and erlotinib effectively target EGFR in individuals with NSCLC, but these therapeutic agents are ultimately limited by the emergence of mutations and other molecular mechanisms conferring drug resistance. MET protein expression and phosphorylation have been associated with both primary and acquired resistance to EGFR TKI therapy in NSCLC patients. There is a need for compositions and methods, such as the control of MET expression, as an effective therapeutic target to overcome resistance to this important class of drugs in lung cancer.

### SEQUENCE LISTING

The instant application contains a Sequence Listing which has been submitted via EFS-web and is hereby incorporated by reference in its entirety. The ASCII copy, created on Dec. 7, 2012, is named 604_{—}53534_SEQ_LIST_{—}2012-111.txt, and is 10,800 bytes in size.

### SUMMARY OF THE INVENTION

The present invention provides compositions comprising at least one nucleic acid selected from the group consisting of: isolated nucleotides 154 through 160 of the miR-221/222 binding site of APAF-1 (5′-ATGTAGC-3′); isolated nucleotides 288 through 294 of the miR-30b binding site of BIM (5′-TGTTTACA-3′); isolated nucleotides complementary to the 27 through 33 of the miR-103 binding site of PKC-ε (complement: 3′-ACGACG-5′); isolated nucleotides complementary to nucleotides 1517 through 1523 of the miR-103 binding site of PKC-ε (complement: 3′-ACGACG); isolated nucleotides complementary to nucleotides 1564 through 1570 of the miR-103 binding site of PKC-ε (complement: 3′-ACGACG-5′); isolated nucleotides complementary to nucleotides 656 through 662 of the miR-203 binding site of SRC (complement: 3′-UAAAGU-5′); isolated nucleotides complementary to nucleotides 1116 through 1122 of the miR-203 binding site of SRC (complement: 3′-UAAAGU-5′); isolated nucleotides complementary to nucleotides 1595 through 1601 of the miR-203 binding site of SRC (complement: 3′-UAAAGU-5′); and isolated nucleotides complementary to nucleotides 1706 through 1712 of the miR-203 binding site of SRC (complement: 3′-UAAAGU-5′).

Also provided are isolated nucleic acids comprising at least one nucleic acid selected from the group consisting of: 5′-ATGTAGC-3′; 5′-TGTTTACA-3′; 3′-ACGACG-5′; and 3′-UAAAGU-5′.

Also provided are isolated nucleic acids or compositions herein, which further comprise an element selected from the group consisting of: promoter; enhancer; repeat; marker; and reporter.

Also provided are isolated nucleic acids herein, which is a probe, primer, miRNA, plasmid, vector, virus, cell, or organism.

Also provided are compositions of matter herein, comprising at least one miR selected from the group consisting of: miR-103; miR-203; anti-miR-30; and anti-miR-221.

Also provided are compositions of matter herein, comprising at least two miR selected from the group consisting of: miR-103; miR-203; anti-miR-30; and anti-miR-221.

Also provided are compositions of matter herein, comprising at least three miR selected from the group consisting of: miR-103; miR-203; anti-miR-30; and anti-miR-221.

Also provided are compositions of matter herein, comprising miR-103; miR-203; anti-miR-30; and anti-miR-221.

Also provided are compositions of matter herein, which further comprise a chemotherapeutic treatment.

Also provided are composition of matter herein, which further comprise a lung cancer chemotherapeutic treatment.

Also provided are composition of matter herein, which further comprise an epidermal growth factor receptor (EGFR) inhibitor.

Also provided are composition of matter herein, which further comprise a tyrosine kinase inhibitor (TKI).

Also provided are composition of matter herein, which further comprise a monoclonal antibody selected from the group consisting of: cetuximab; panitumumab; zalutumumab; nimotuzmab; and matuzumab.

Also provided are composition of matter herein, which further comprise a small molecule selected from the group consisting of: gefitinib; erlotinib; lapatinib; AP26113; and potato carboxypeptidase inhibitor.

Also provided are composition of matter herein, which further comprise getifitinib.

Also provided are composition of matter herein, which further comprise a PKC-ε expression agonist.

Also provided are composition of matter herein, which further comprise a MET inhibitor.

Also provided are composition of matter herein, which further comprise SU11274.

Also provided are composition of matter herein, which further comprise a DICER inhibitor.

Also provided are composition of matter herein, which further comprise a E-cadherin expression agonist.

Also provided are composition of matter herein, which further comprise an adjuvant, excipient, and/or other pharmaceutically-acceptable compositions.

Also provided are composition of matter herein, formulated for injection, transfusion, ingestion or transmembrane conveyance.

The present invention provides methods downregulate DICER in a mammalian cell, comprising increasing miR-103 and/or miR-203 availability in a mammalian cell, and downregulating DICER in the mammalian cell.

The present invention provides methods to decrease migration in a mammalian cancer cell, comprising increasing miR-103 and/or miR-203 availability in a mammalian cancer cell, and decreasing migration of the mammalian cancer cell.

The present invention provides methods to decrease EGFR chemotherapy resistance of a mammalian cancer cell, comprising increasing miR-103 and/or miR-203 availability in a mammalian cancer cell, and decreasing EGFR chemotherapy resistance of the mammalian cancer cell.

The present invention provides methods to decrease gefitinib resistance a mammalian cancer cell, comprising increasing miR-103 and/or miR-203 availability in a mammalian cancer cell, and decreasing gefitinib resistance of the mammalian cancer cell.

The present invention provides methods to decrease expression of mesenchymal markers in a mammalian cancer cell, comprising increasing miR-103 and/or miR-203 availability in a mammalian cancer cell, and decreasing expression of mesenchymal markers of the mammalian cancer cell.

The present invention provides methods to increase expression of E-cadherin expression in a mammalian cancer cell, comprising increasing miR-103 and/or miR-203 availability in a mammalian cancer cell, and increasing expression of E-cadherin expression of the mammalian cancer cell.

The present invention provides methods to induce mesenchymal-epithelial transition in a mammalian cancer cell, comprising increasing miR-103 and/or miR-203 availability in a mammalian cancer cell, and inducing mesenchymal-epithelial transition of the mammalian cancer cell. Such methods wherein the mesenchymal-epithelial transition is induced through PKC-ε and/or DICER are provided.

The present invention provides methods to induce programmed cell death in a mammalian cancer cell, comprising increasing miR-103 and/or miR-203 availability in a mammalian cancer cell, and inducing programmed cell death of the mammalian cancer cell.

The present invention provides methods to downregulate AKT/ERK in a mammalian cancer cell, comprising increasing miR-103 and/or miR-203 availability in a mammalian cancer cell, and inducing mesenchymal-epithelial transition of the mammalian cancer cell.

The present invention provides methods to increase gefitinib sensitivity in a mammalian cancer cell, comprising increasing miR-103 and/or miR-203 availability in a mammalian cancer cell, and increasing gefitinib sensitivity of the mammalian cancer cell.

Such methods wherein the cancer cell is a lung cancer cell are provided.

Such methods wherein the cancer cell is a non-small cell lung adenocarcinoma cell are provided.

Such methods wherein the cancer cell is an epidermal carcinoma cell are provided.

The present invention provides methods to inhibit tumor growth in a mammal in need of tumor growth inhibition, comprising administering tumor growth-inhibiting amount of at least one nucleic acid selected from the group consisting of: isolated nucleotides 154 through 160 of the miR-221/222 binding site of APAF-1 (5′-ATGTAGC-3′); isolated nucleotides 288 through 294 of the miR-30b binding site of BIM (5′-TGTTTACA-3′); isolated nucleotides complementary to the 27 through 33 of the miR-103 binding site of PKC-ε (complement: 3′-ACGACG-5′); isolated nucleotides complementary to nucleotides 1517 through 1523 of the miR-103 binding site of PKC-ε (complement: 3′-ACGACG); isolated nucleotides complementary to nucleotides 1564 through 1570 of the miR-103 binding site of PKC-ε (complement: 3′-ACGACG-5′); isolated nucleotides complementary to nucleotides 656 through 662 of the miR-203 binding site of SRC (complement: 3′-UAAAGU-5′); isolated nucleotides complementary to nucleotides 1116 through 1122 of the miR-203 binding site of SRC (complement: 3′-UAAAGU-5′); isolated nucleotides complementary to nucleotides 1595 through 1601 of the miR-203 binding site of SRC (complement: 3′-UAAAGU-5′); and isolated nucleotides complementary to nucleotides 1706 through 1712 of the miR-203 binding site of SRC (complement: 3′-UAAAGU-5′).

The present invention provides methods to inhibit tumor growth in a mammal in need of tumor growth inhibition, comprising administering a tumor growth-inhibiting amount of a nucleic acid selected from the group consisting of: 5′-ATGTAGC-3′; 5′-TGTTTACA-3′; 3′-ACGACG-5′; and 3′-UAAAGU-5′.

The present invention provides methods to inhibit tumor growth in a mammal in need of tumor growth inhibition, comprising administering tumor growth-inhibiting amount of at least one miR selected from the group consisting of: miR-103; miR-203; anti-miR-30; and anti-miR-221.

The present invention provides methods to inhibit tumor growth in a mammal in need of tumor growth inhibition, comprising administering tumor growth-inhibiting amount of at least one miR selected from the group consisting of: miR-103; miR-203; anti-miR-30; and anti-miR-221.

The present invention provides methods to inhibit tumor growth in a mammal in need of tumor growth inhibition, comprising administering tumor growth-inhibiting amount of at least one miR selected from the group consisting of: miR-103; miR-203; anti-miR-30; and anti-miR-221.

The present invention provides methods to inhibit tumor growth in a mammal in need of tumor growth inhibition, comprising administering tumor growth-inhibiting amount of: miR-103; miR-203; anti-miR-30; and anti-miR-221.

The present invention provides such methods which further comprise administering a chemotherapeutic treatment.

The present invention provides such methods which further comprise comprises administering lung cancer chemotherapeutic treatment.

The present invention provides such methods which further comprise administering an epidermal growth factor receptor (EGFR) inhibitor.

The present invention provides such methods which further comprise administering a tyrosine kinase inhibitor (TKI).

The present invention provides such methods which further comprise administering a monoclonal antibody selected from the group consisting of: cetuximab; panitumumab; zalutumumab; nimotuzmab; and matuzumab.

The present invention provides such methods which further comprise administering a small molecule selected from the group consisting of: gefitinib; erlotinib; lapatinib; AP26113; and potato carboxypeptidase inhibitor.

The present invention provides such methods which further comprise administering gefitinib.

The present invention provides such methods which further comprise administering a PKC-ε expression agonist.

The present invention provides such methods which further comprise administering a MET inhibitor.

The present invention provides such methods which further comprise administering SU11274.

The present invention provides such methods which further comprise administering a DICER inhibitor.

The present invention provides such methods which further comprise administering an E-cadherin expression agonist.

The present invention provides such methods which further comprise administering an adjuvant, excipient, and/or other pharmaceutically-acceptable compositions.

The present invention provides such methods wherein administration is via injection, transfusion, ingestion or transmembrane conveyance.

The present invention provides such methods wherein the tumor is a lung tumor.

The present invention provides such methods wherein the tumor is a lung carcinoma.

The present invention provides such methods wherein the tumor is a lung adenocarcinoma.

The present invention provides such methods wherein the tumor is non-small cell lung carcinoma.

The present invention provides such methods wherein the tumor growth is reduced by at least 10%, at least 20% at least 30%, at least 40%, at least 50% and at least 60% compared to control.

The present invention provides methods to promote wound healing in a mammal in need of wound healing promotion, comprising administering a wound healing-promoting amount of a composition herein.

The present invention provides kits comprising a composition herein.

The present invention provides cells comprising a composition herein.

The present invention provides a mouse comprising a composition herein.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1J. TKI-regulated miRNA targets.

FIG. 1A. Downregulation of EGFR and MET proteins and mRNAs after EGFR and MET silencing.

FIG. 1B. Unsupervised hierarchical clustering in Calu-1 cells after knockdown of EGFR (shEGFR), MET (shMET) or shCtr (scrambled RNA). CTR, control. P<0.05.

FIG. 1C. Intersection of miRNAs regulated by shEGFR and shMET.

FIG. 1D. Northern blots showing deregulated miRNAs after shMET. snRNA U6, loading control.

FIG. 1E. Luciferase report assays indicated direct interactions between the miRNAs and PRKCE (PKC-ε), SRC, APAF1 and BCL2L11 (BIM) 3′ UTRs. In SRC only, the site at 1,595-1,601 nt is implicated in binding with miR-203; deletion of the site at 1,706-1,712 nt did not rescue luciferase activity (FIG. 8). WT, wild type; MUT, mutated; scr, scrambled.

FIG. 1F. Inverse correlation between miR-103, miR-203, miR-221, miR-222, miR-30b and miR-30c and target proteins in a panel of NSCLC cells.

FIG. 1G. miR-221, miR-222, miR-30b and miR-30c overexpression decreased the concentration of APAF-1 and BIM proteins.

FIG. 1H. miR-103 and miR-203 overexpression decreased the concentration of PKC-ε and SRC proteins.

FIG. 1I. Inhibitors of miR-221, miR-222, miR-30b and miR-30c increased APAF-1 and BIM expression.

FIG. 1J. shMET induced upregulation of APAF-1 and BIM and downregulation of SRC and PKC-ε. Results are representative of at least three independent experiments. Error bars, ±s.d * P<0.001, ** P<0.05 by two-tailed Student's t test.

FIGS. 2A-2D. MET-miRNA coexpression analysis.

FIG. 2A. One hundred ten lung cancer tissues were analyzed for miR-103, miR-222, miR-203 and miR-30c expression by ISH and then for MET by IHC. The top row shows the miR-103 signal (blue), the MET signal (red) and the mixed signal, in which fluorescent yellow indicates miRNA and protein co-expression; there is a lack of miR-103 in the presence of MET expression. In the serial section of the same cancer (in the second row), miR-222, the MET image and the co-expression of miR-222 and MET are shown. Many cancer cells positive for miR-222 also express MET (yellow). The arrows in the left panel (in the third row) point to benign stromal cells that express miR-203 (blue) and not MET. The other images in the third row show the MET signal (red) and the mixed signal. The arrow in the left image in the fourth row points to cancer cells positive for miR-30c. The right images in each row show the RGB image of the ISH or IHC reaction.

FIG. 2B. Box plots showing miRNA expression in 40 individuals with lung cancer. Real-time PCR was used to classify tumors into two groups, EGFR-MET low and EGFR-MET high, using a round function with a cutoff of 0.5 (2^{(-ΔCT)}). *P<0.0001 by Student's t test.

FIG. 2C. XY scatter plots showing inverse correlation between MET and miR-103 and MET and miR-203.

FIG. 2D. MET and EGFR IHC on 40 lung tumor tissues. One representative case from 17 metastatic tumors expressing both MET and EGFR is shown. The large green arrows point to the tumor cells, and the small black arrows point to the stroma. Scale bars, 100 μm.

FIGS. 3A-3D. Gefitinib downregulates miR-221, miR-222, miR-30b and miR-30c.

FIG. 3A. Calu-1, A549, PC9 and HCC827 cells were treated with increasing concentrations of gefitinib. Cell viability, relative to untreated controls, was measured after 24 h. Each data point represents the mean±s.d. of five wells.

FIG. 3B. qRT-PCR showing miR-30b, miR-30c, miR-221 and miR-222 downregulation only in PC9 and HCC827 gefitinib-sensitive cells and not in Calu-1 and A549 gefitinib-resistant cells after treatment with 5 μM or 10 μM gefitinib. NT, non-treated cells.

FIG. 3C. PC9, Calu-1 and HCC827 cells were treated for 24 h with 5 μM or 10 μM gefitinib. An increase of BIM and APAF-1 expression and a decrease of the phosphorylation of the ERKs were observed only in the HCC827 and PC9 gefitinib-sensitive cells but not in the Calu-1 gefitinib-resistant cells. β-actin was used as a loading control.

FIG. 3D. qRT-PCR showing that miR-221, miR-222, miR-30b and miR-30c expression did not decrease in HCC827 GR and PC9 GR cells (cells with acquired gefitinib resistance) exposed to 10 μM gefitinib for 24 h. All quantitative data were generated from a minimum of three replicates. Error bars, ±s.d. A two tailed Student's t test was used to determine the P values. *P<0.001, **P<0.05.

FIGS. 4A-4F. miR-30b, miR-30c, miR-221, miR-222, miR-103 and miR-203 regulate gefitinib sensitivity.

FIG. 4A. Parental and their resistant HCC827 GR Q27Q16 and PC9 GR and Calu-1 clone cells treated with increasing concentrations of gefitinib. Each data point represents the mean±s.d of six wells.

FIG. 4B. A western blot showing an increase in gefitinib-induced cleaved PARP fragments after overexpression of BIM and APAF-1 and after treatment with gefitinib (15 μM) in A549 cells. EV, empty virus.

FIG. 4C. Silencing of BIM (siBAM) and APAF-1 (siAPAF-1) in HCC827 and PC9 cells reduces the response to gefitinib.

FIG. 4D. Overexpression of BIM and APAF-1 complementary DNAs insensitive to miR-30b, miR-30c, miR-221 and miR-222 induces gefitinib sensitivity in A549 cells. SiScr, SRC siRNA.

FIGS. 4E-4F. Overexpression of miR-103 and miR-203 and silencing of miR-30c and miR-222 increase gefitinib sensitivity in vivo. Growth curve of engrafted tumors (FIG. 4D) and comparison of engrafted tumors (FIG. 4F) in nude mice injected with A549 cells stably infected with inhibitors of control miRNAs (ctr), miR-30c or miR-221 and with miR-103 and miR-203 or an empty virus as a control. The images show average-sized tumors from among five tumors from each category. In FIG. 4A and FIGS. 4A-4D, error bars, ±s.d. *P<0.001, **P<0.05 by two-tailed Student's t test. Gef, gefitinib.

FIGS. 5A-5C. MiR-103 and miR-203 inhibit the migration and proliferation of NSCLCs.

FIG. 5A. Representative images of cells that migrated through the filter and that were stained with crystal violet. Scale bar, 40 μm. The results are means±s.d. n=3 experiments. *P<0.001.

FIG. 5B. Representative photographs of scratched areas of the confluent monolayer of A549 cells transfected with miR-103, miR-203 or control miRNA (Scr miR) at 0 h and 24 h after wounding with a pipet tip. Scale bar, 500 *P<0.00001, **P<0.001, relative to miRNA scrambled transfected cells.

FIG. 5C. Flow cytometric distributions of Calu-1 and A549 cells transfected with control miRNAs, miR-103 (103), miR-203 (203), control siRNA (Scr siRNA) and siRNAs of PKC-ε (siPKC-ε) and SRC (siSRC). The effect of miR-203 on cell cycle is slightly stronger than that of miR-103, as assessed by the ratio between the G0-G1 and S phases. All quantitative values show mean±s.d. n=5. A two tailed Student's t test was used to determine the P values for the G0-G1:S ratios. *P<0.00001 and **P<0.005, compared to scrambled miRNA.

FIGS. 6A-6H. MET induces epithelial-mesenchymal transition.

FIG. 6A. Morphological changes of Calu-1 cells after MET knockdown. Scale bar, 20 μm.

FIG. 6B. Immunofluorescence of Snail, vimentin and N-cadherin in Calu-1 shCtr cells and Calu-1 shMET cells. Snail expression is strong and nuclear in Calu-1 shCtr cells and is weaker and cytoplasmic in Calu-1 shMET cells. Scale bars, 20 μm.

FIG. 6C. Western blots showing the fibronectin, vimentin and Snail downregulation and the upregulation of E-cadherin after MET knockdown in Calu-1 cells. Loading control, GAPDH.

FIG. 6D. qRT-PCR showing the expression of epithelial and mesenchymal markers in Calu-1 shCtr cells and Calu-1 shMET cells.

FIG. 6E. Immunofluorescence showing that fibronectin, Snail and vimentin expression decreases after miR-103 or miR-203 overexpression in Calu-1 cells. Scale bar, 20 μm. Ctr miR, control miRNA.

FIG. 6F. Immunofluorescence showing the increased E-cadherin signal after miR-103- or miR-203-enforced expression in Calu-1 cells. Scale bar, 40 μm.

FIG. 6G. Immunoblot showing the downregulation of mesenchymal markers after miR-103 or miR-203 overexpression.

FIG. 611. Model in which MET downregulates miR-103 and miR-203, which in turn, upreggate PKC-ε, Dicer and SRC, inducing gefitinib resistance and epithelial-mesenchymal transition. MET also induces miR-30b, miR-30c, miR-221, miR-222 and miR-21 upregulation and the consequent gefitinib resistance through BIM, APAF-1 and PTEN downregulation. EGFR increases miR-221, miR-222, miR-30b and miR-30c expression. Shown in red are the upregulated miRNAs, and shown in green are the downregulated miRNAs. Results are representative of at least four independent experiments. P values, two-tailed Student's t test. Error bars, ±s.d.

FIGS. 7A-7B. MicroRNAs deregulated after stable EGFR and MET silencing.

FIG. 7A. MicroRNAs deregulated after EGFR (Table 1) and MET (Table 2) silencing, with 1.5- (EGFR) and with 1.7 (MET)-fold changes are shown (P<0.05). Green=downregulated miRs; Red=upregulated miRs.

FIG. 7B. qRT-PCR showing miR-221/miR-222 and -30b/c downregulation after MET and EGFR silencing and miR-103 and -203 upregulation after MET silencing. Data are means±s.d. of three independent experiments. *P<0.001, **P<0.0001.

FIGS. 8A-8D. miR-221/miR-222, miR-30b-c, miR-103 and miR-203 predicted targets.

FIG. 8A. APAF-1 3′UTR presents one miR-221/miR-222 binding site (nucleotides 154-160 (SEQ ID NO: 38)); BIM presents one miR-30b/c binding site (nt 288-294 (SEQ ID NO: 42)); PKC-ε presents three miR-103 binding sites (nt 27-33 (SEQ ID NO: 39), 1517-1523 (SEQ ID NO: 40), 1564-1570 (SEQ ID NO: 41)); SRC 3′UTR presents four miR-203 binding sites (nt 656-662 (SEQ ID NO: 43), 1116-1122 (SEQ ID NO: 44), 1595-1601 (SEQ ID NO: 45), 1706-1712 (SEQ ID NO: 46)). In the figure the alignment of the seed regions of miR-221 and miR-222 with APAF-1, miR-30b/c with BIM, miR-103 with PKC-ε and miR-203 with SRC 11 3′UTRs is shown. The sites of target mutagenesis are indicated in green: =deleted nucleotides. 370 and 342 bp of the 3′ UTRs for APAF-1 and BIM were amplified, respectively. Three different constructs for PKC-ε (27-33 (bp=385), 1517-1570 (bp=496), 27-1570 (bp=1720)) and two for SRC (656-1122 (bp=705), 1595-1712 (bp=805)) were generated. BS=binding site.

FIG. 8B. Western blots in a panel of 7 NSCLC cells. Protein abundance is reported as western blotting densitometry normalized to β-actin expression.

FIG. 8C. qRT-PCR showing low expression of miR-103, miR-203, as compared with miR-221/miR-222, miR-30b/c relative expression levels, in a panel of NSCLC cells.

FIG. 8D. The association between miR-103, miR-203, miR-30b/c, miR-221/miR-222 and PKC-ε, SRC, BIM and APAF-1 mRNAs in the 7 NSCLC cells was calculated statistically by using the Pearson Correlation Coefficient (r) and the respective p-values, all significant at P<0.01. The Pearson correlation indicated an inverse relation between miR-103, miR-203, miR-30c, miR-222 and PKC-s, SRC, BIM and APAF-1 mRNAs in all the cells analyzed. Results are representative of at least, three independent experiments. Error bars depict ±s.d.

FIGS. 9A-9B. miR-221/miR-222, miR-30b/c, miR-103, miR-203 target APAF-1, BIM, PKC-H and SRC.

FIG. 9A. Calu-1 MET-KD cells, transfected with miR-221/miR-222 and miR-30b/c, present a decrease in APAF-1 and BIM protein levels.

FIG. 9B. Conversely, anti-miR-103 and miR-203 increase PKC-ε and SRC expression, respectively, Scr=scrambled. Results are representative of at least three, independent experiments.

FIGS. 10A-10B. miR-103-PKC-ε, miR-222-APAF-1, miR-203-SRC and miR-30c-BIM co-expression analyses.

FIG. 10A. 110 lung cancer tissues were analyzed for miR-103, miR-222, miR-203, miR-30c expression by ISH and then for PKC-ε, APAF-1, SRC and BIM by IHC. Upper row, from the left miR-103 (blue) and PKC-H (red) results show a weak signal for the miRNA and a strong signal for the protein in this lung cancer. Mixing of the images (third panel) shows no co-expression of the two targets, which would appear as yellow; note the localization of the PKC-ε signal (red) to the nests of cancer cells (arrows). Second row, left panel is a strong miR-222 signal and a weak signal for the putative target APAF-1 in the cancer cells (large arrow, second panel), but not the surrounding benign stromal cells (small arrow). Third panel shows no detectable co-expression. Third row, left panel is miR-203 (blue), next is the SRC signal (red) and the mixed signal; note the lack of miR-203 and SRC co-expression. In the right panel the counterstain hematoxylin is added as fluorescent turquoise. This allows one to see that the cancer cells (large arrow) are expressing SRC and not the benign desmoplastic cells (small arrow). Last row, left panel is miR-30c signal (blue), next BIM (red) and the merged image where the lack of yellow indicates no co-expression of the two targets. Right panels show the regular color-based image (RGB=Red, Green, Blue). Scale bar indicates 100 Pm.

FIG. 10B. Tables showing the inverse relation between microRNAs and protein targets expression in 110 lung tumors.

FIG. 11A-11E. MET is overexpressed in metastatic lung tumor tissues.

FIG. 11A. Table reporting the percentage of MET and miR-30c, miR-103, miR-203, miR-222 expression observed in the 110 tumor samples analyzed. miR-103 and miR-203 are inversely correlated and miR30c and -222 directly correlated to MET expression in the majority of the tumor specimens.

FIG. 11B. Percentage of metastatic and non-metastatic lung tumor samples expressing MET. MET is overexpressed in the metastatic tumors compared to the lung non-metastatic tissues. P=0.021 by Fisher's exact test.

FIG. 11C. 40 lung tumors were divided in “high” and “low” EGFR and MET expression by qRT-PCR by round function with the cutoff at 0.5 (2^{(-DeltaCt)}).

FIG. 11D. 2×2 contingency table showing the association between IHC analysis and qRT-PCR results for EGFR and MET. P<0.0001 by Fisher exact test.

FIG. 11E. Tables showing the number of metastatic tumors expressing MET and EGFR in the 40 lung cancers. Note the direct relation between metastases and MET but not EGFR expression levels. MET, P=0.026; EGFR, P=not significant by Fisher's exact test.

FIGS. 12A-12B. APAF-1 and BIM expression in PC9GR and HCC827GR cells. HCC827GR cells (FIG. 12A) and PC9GR cells (FIG. 12B) were treated with 5 or 10 μM gefitinib for 24 h. APAF-1 and BIM expression and ERKs phosphorylation did not change after gefitinib treatment, as a consequence of miR-221/miR-222 and miR-30b/c unchanged expression. B-actin was used a loading control.

FIGS. 13A-13C. miR-30b, miR-30c, miR-221, miR-222 are involved in gefitinib-induced apoptosis.

FIG. 13A. Enforced expression of miR-30b, miR-30c, miR-221, miR-222 increases resistance to gefitinib induced apoptosis in HCC827 and PC9 sensitive cells as assessed by caspase 3/7 assay.

FIG. 13B. miR-30b, miR-30c, miR-221, miR-222 knockdown increases gefitinib sensitivity in NSCLC cells with de novo (Calu-1) and acquired (HCC827GR and PC9GR) resistance to TKIs.

FIG. 13C. A549 were cotransfected with miR-30b/c, miR-221/miR-222 and APAF-1 and BIM cDNAs followed by their 3′UTRs, containing the WT or mutated miRNA binding sites. Overexpression of miR-30b/c- and miR-221/miR-222-insensitive BIM and APAF-1 cDNAs, induces gefitinib sensitivity in A549 cells by MTS assay. All experiments were performed at least three times with essentially identical results. One representative of three independent experiments is shown. Two tailed student's t test was used to determine P values. Error bars depict ±s.d. *P<0.001, **P<0.05.

FIGS. 14A-14D. MET inhibition induces down-regulation of miR-30b-c and miR-221/miR-222.

FIG. 14A. qRT-PCR showing miR-30b/c and miR-221/miR-222 down-regulation after treatment of Calu-1 cells with SU11274. Cells were treated with the MET inhibitor for 24, 48 and 72 h at a concentration of 1 and 3 μM. RNA extraction and qRT-PCR were performed, as described herein. Results from three different experiments are shown.

FIG. 14B. Northern blots showing miR-30c and miR-222 down-regulation in A549 cells after MET KD. SnRNA U6 was used as loading control.

FIG. 14C. Calu-1 cells were treated with the MET inhibitor SU11274. After 24 h cells were exposed to gefitinib (5-10-10-20) μM) for 24 h. MET inhibition increased Caluu-1 sensitivity to the drug as assessed by MTS assay.

FIG. 14D. Calu-1-MET knockdown cells (Calu-MET-KD) treated with gefitinib (5-10-15-20 μM) for 24 h were more sensitive to gefitnib as assessed by caspase 3/7 assay. Experiments were performed three times in triplicate. Error bars represent standard deviation. Two-tailed t test was used to determine all P values. *P<0.001.

FIGS. 15A-15B. EGFR and MET regulated miRNAs involved in gefitinib resistance. qRT_PCT showing miR-21, miR-29a, miR-29c and miR-100 dowregulation in HCC827 and PC9, but not in HCC827GR and PC9GR cells after treatment with 5 and 10 μM gefitinib. Relative values are shown as mean and ±s.d. Two tailed student's t test was used to determine P values. *P<0.005, **P<0.001.

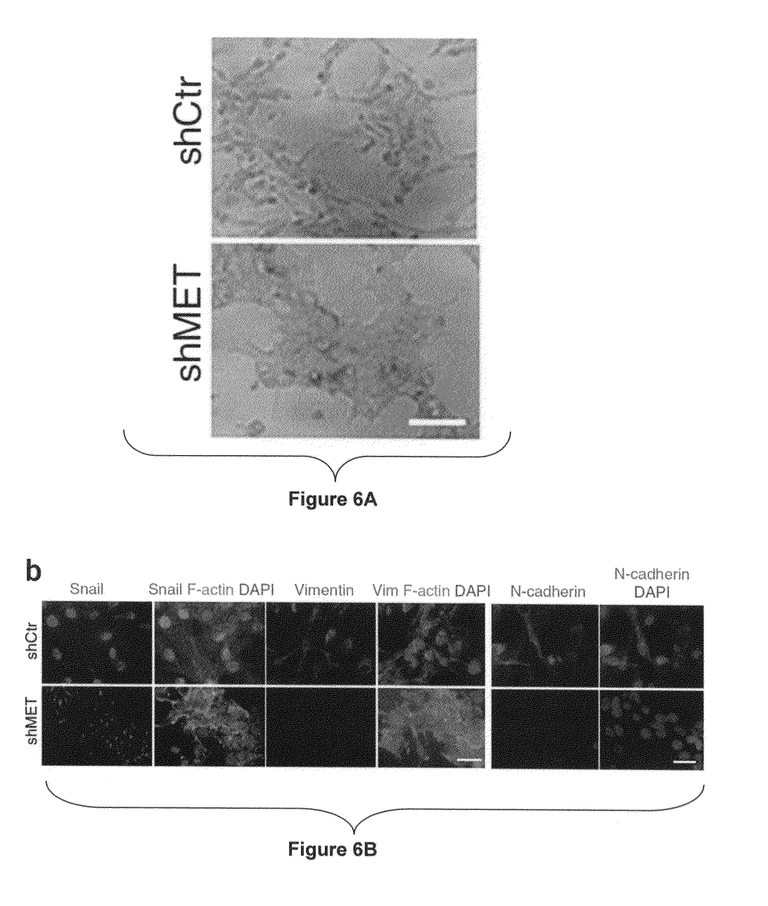

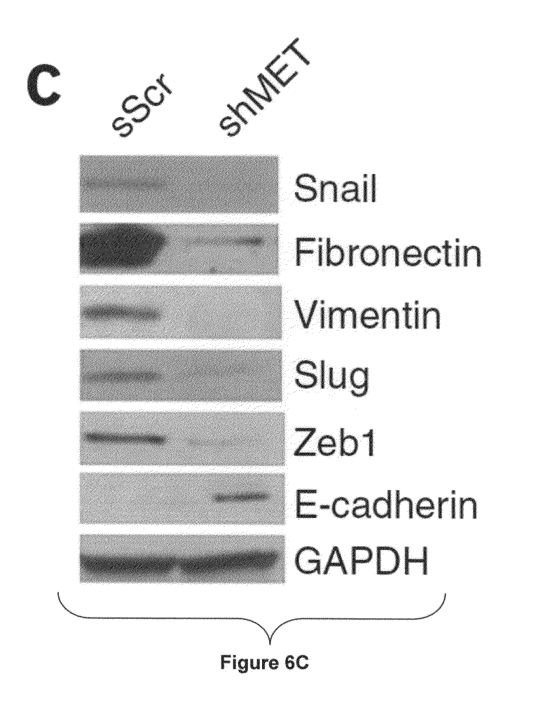

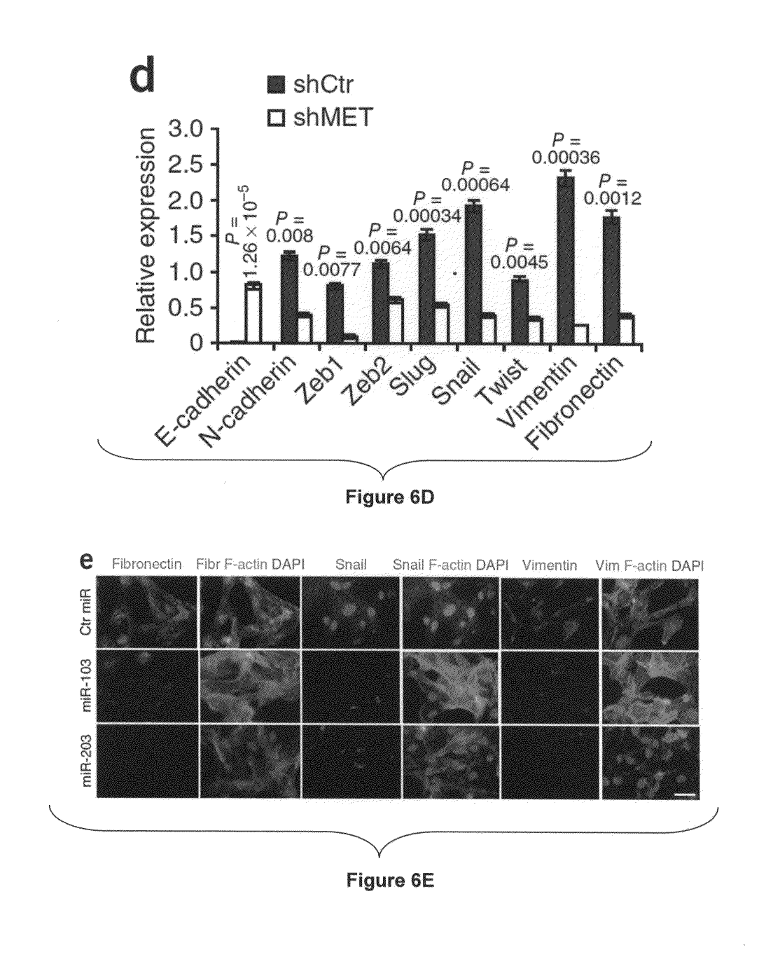

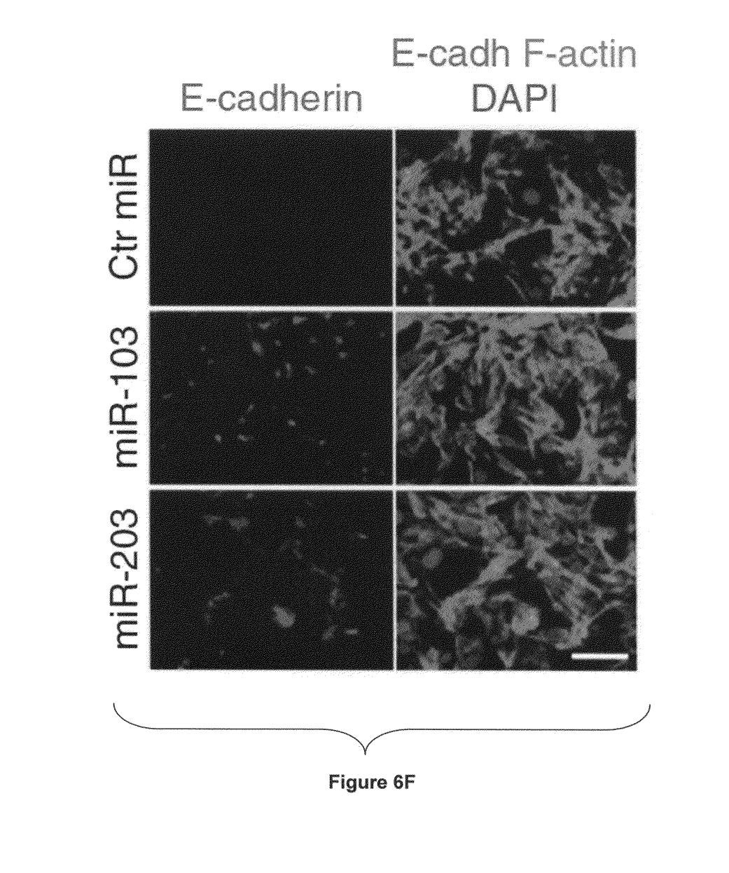

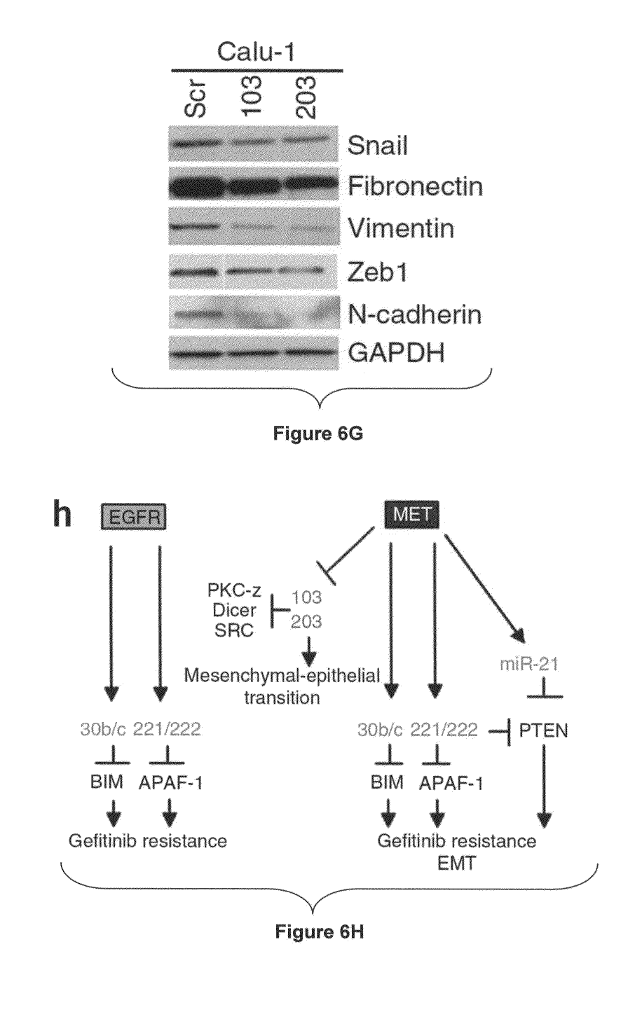

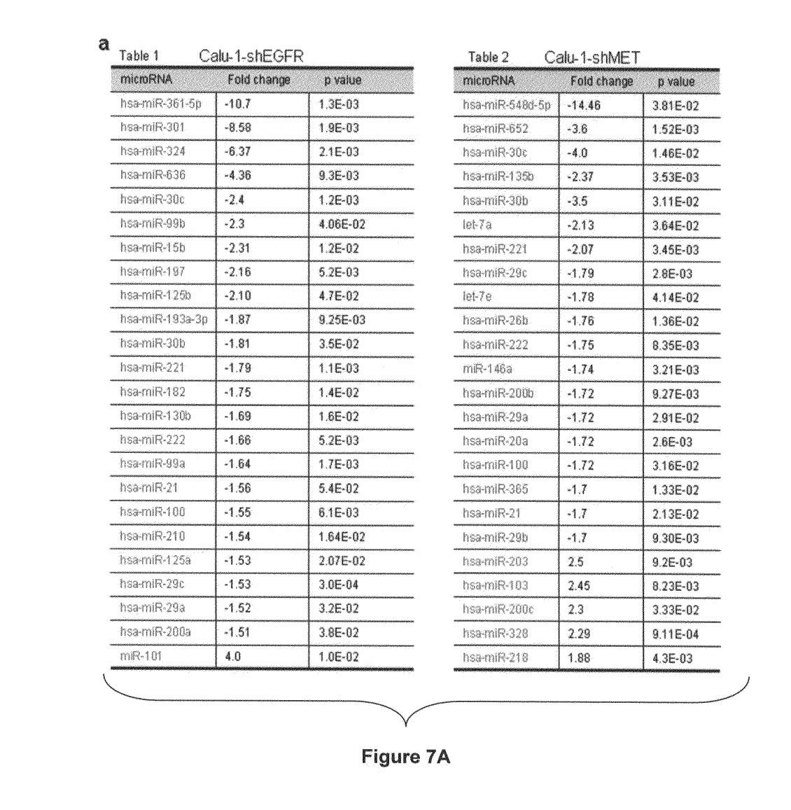

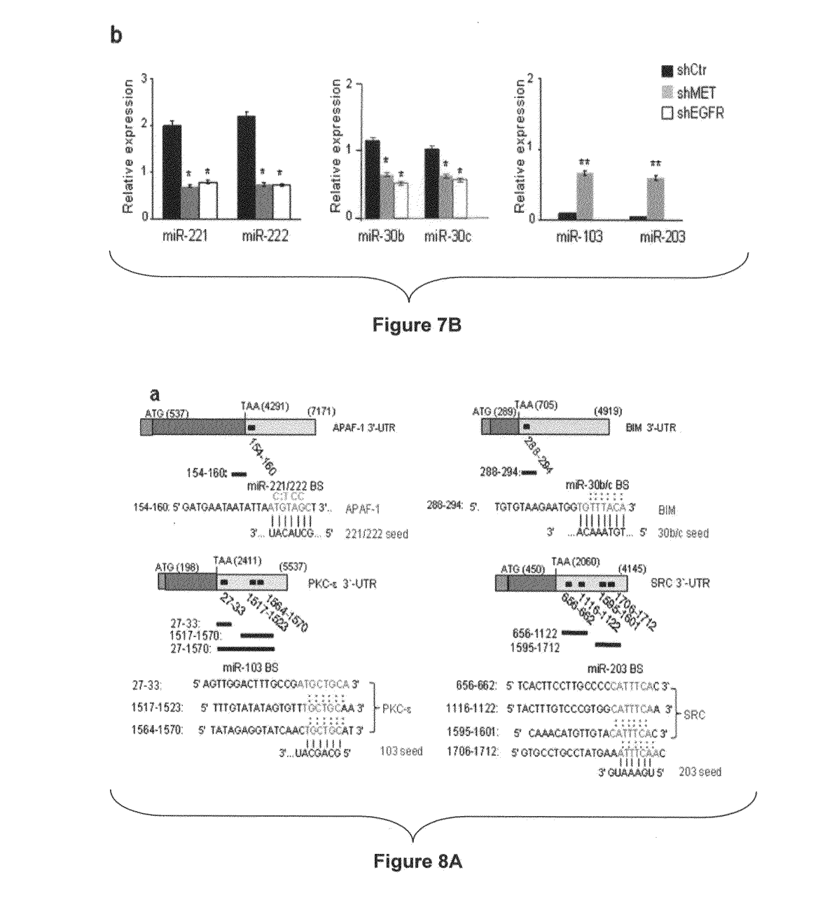

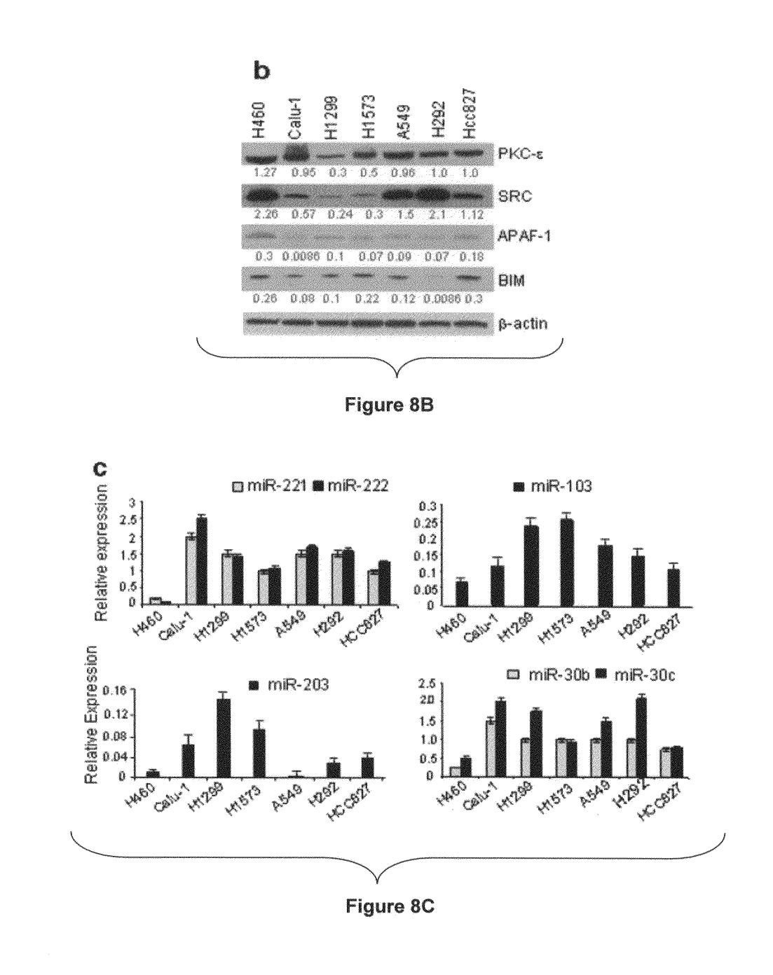

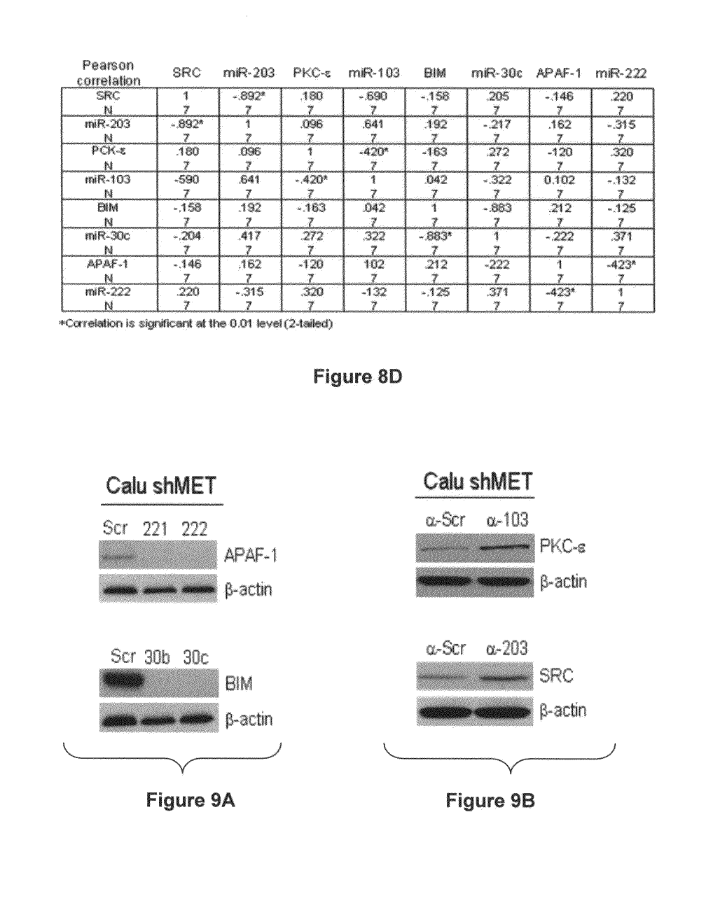

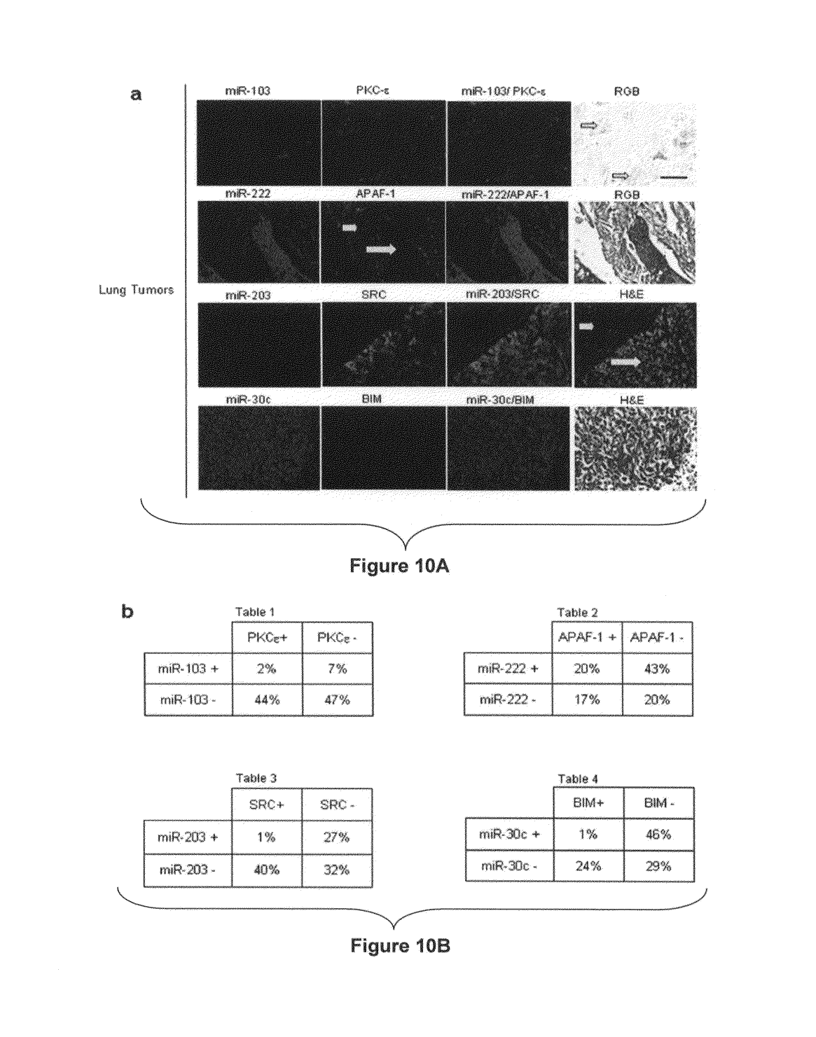

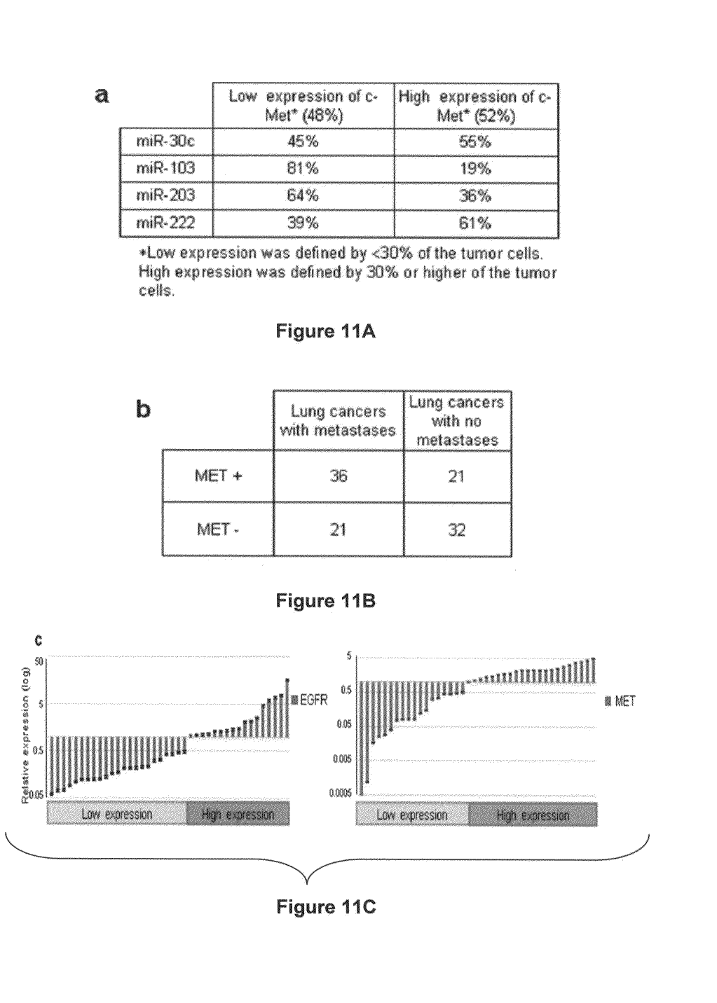

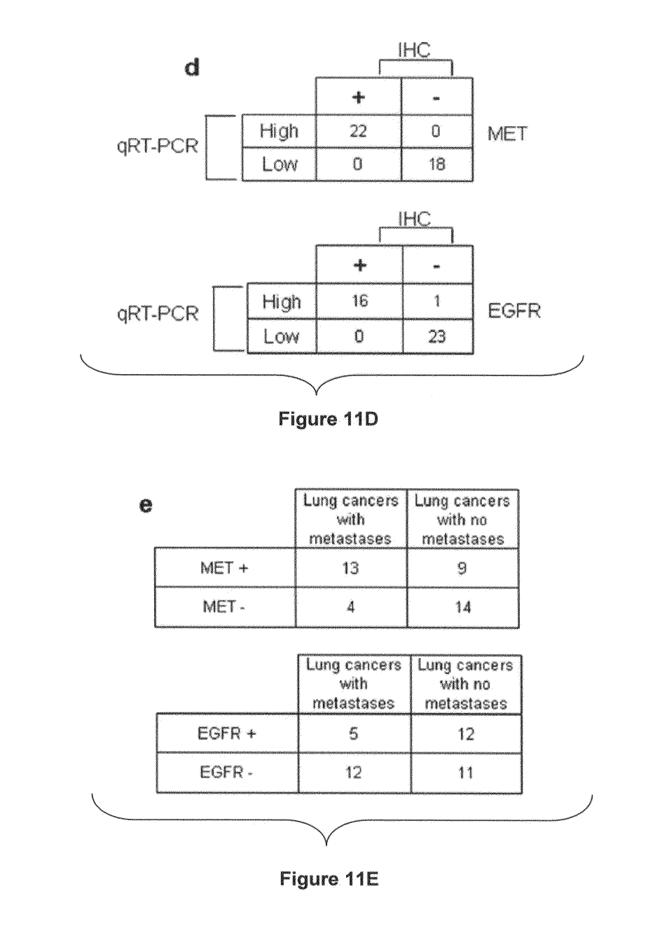

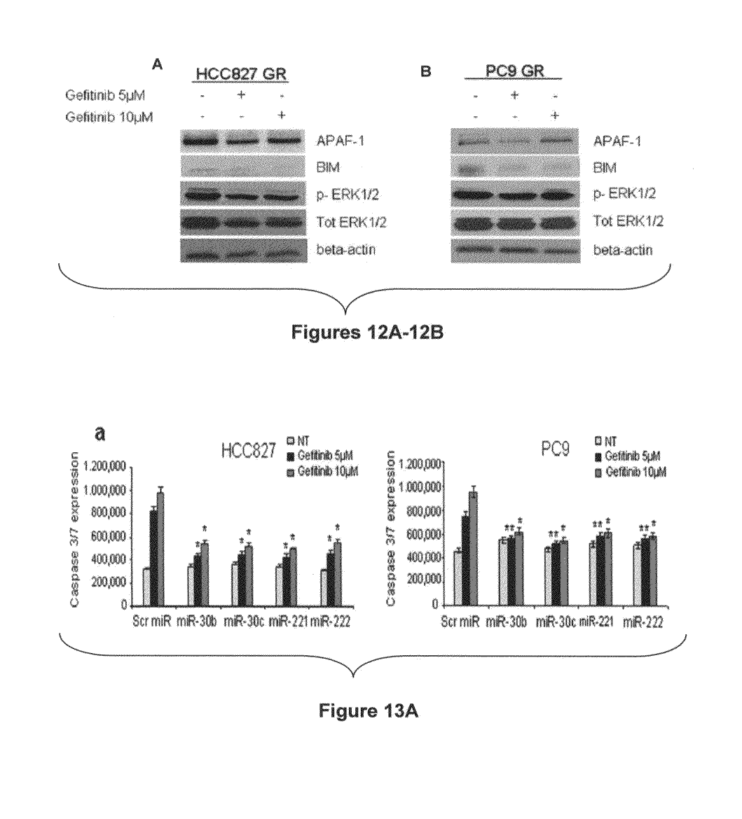

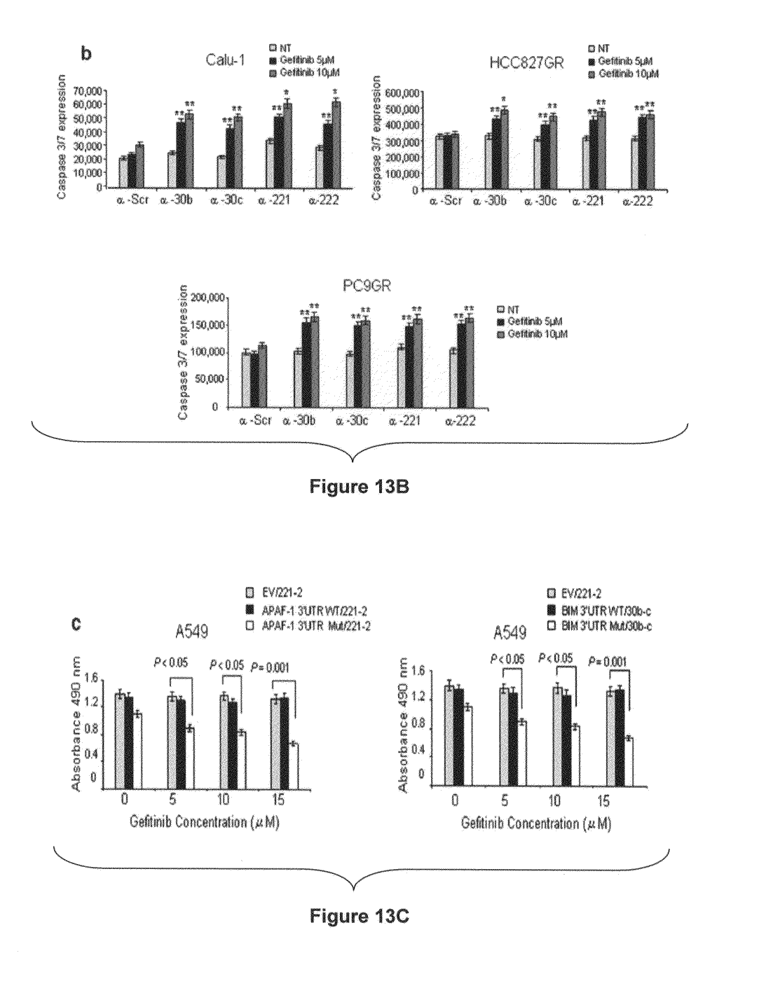

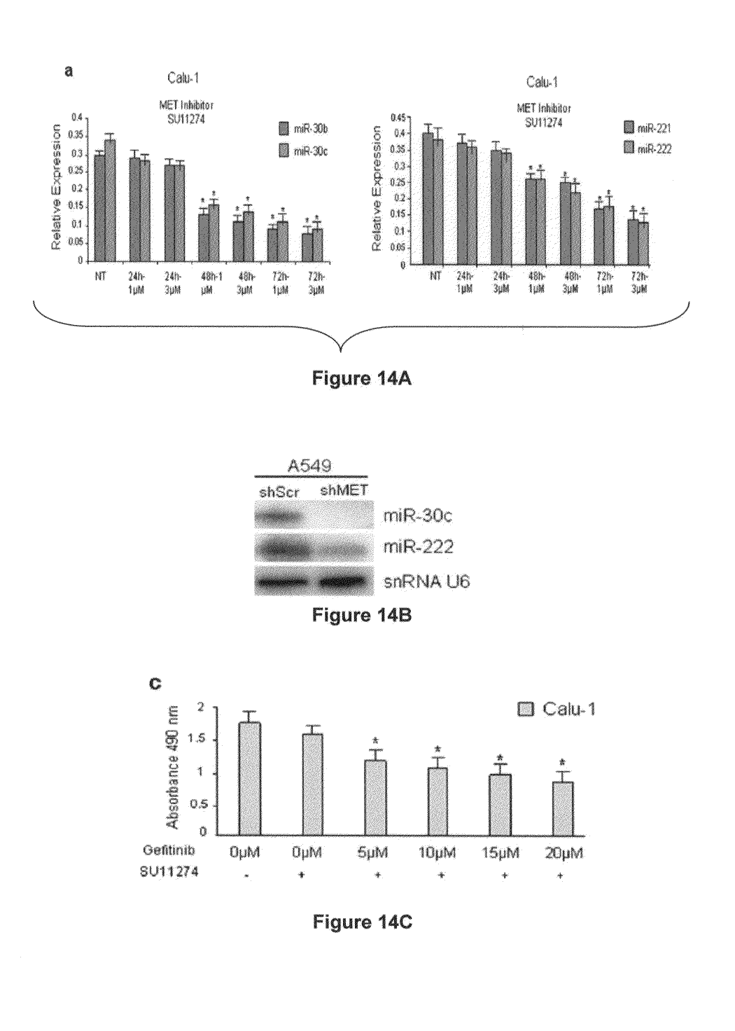

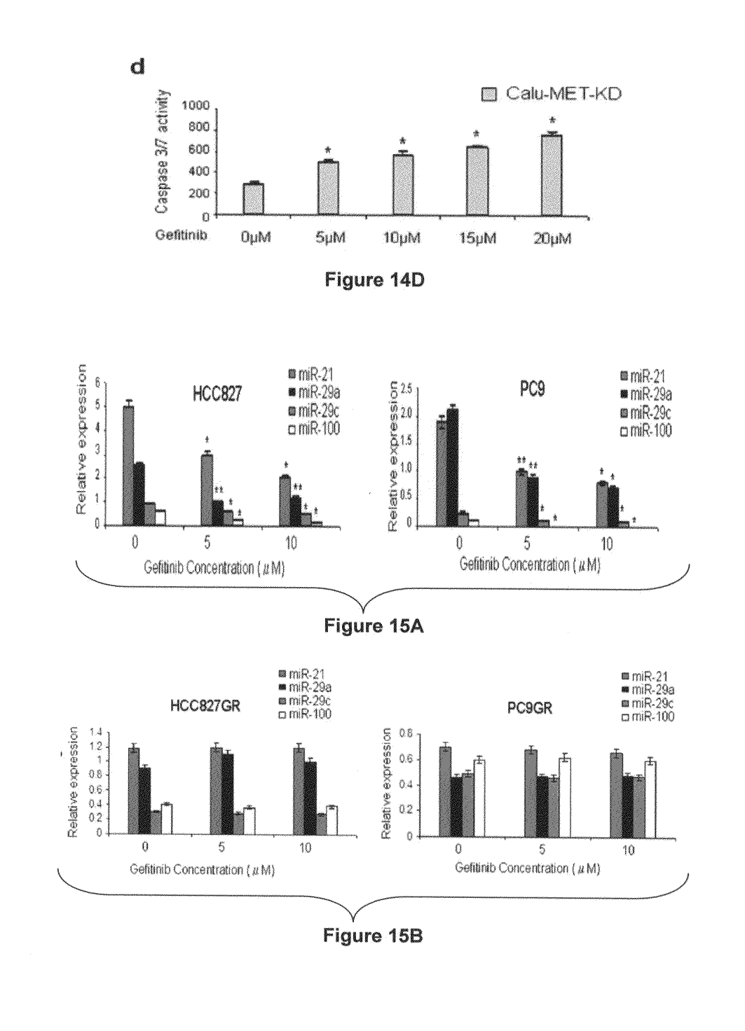

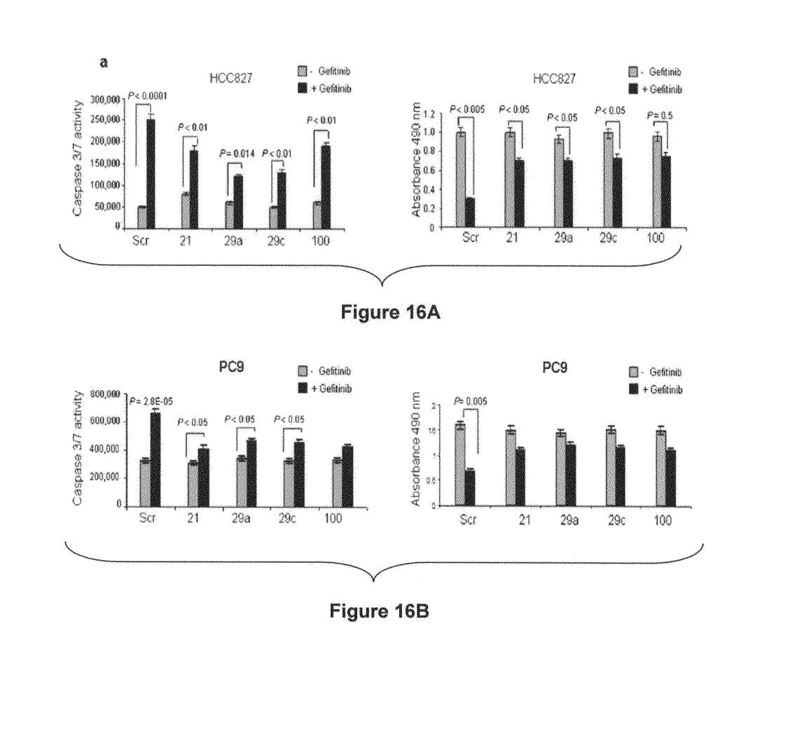

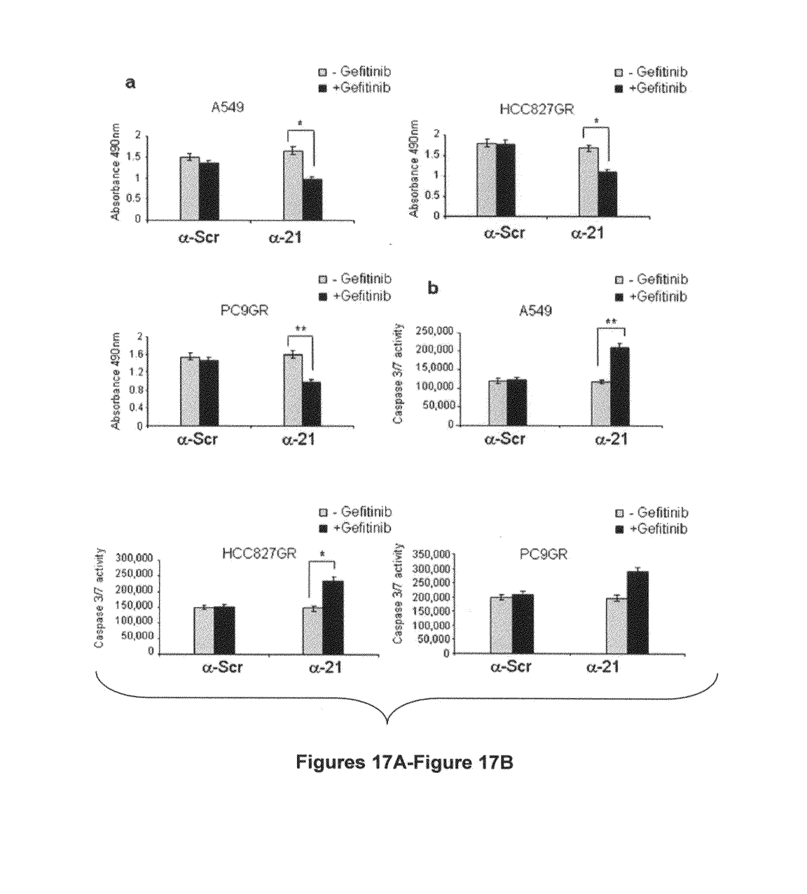

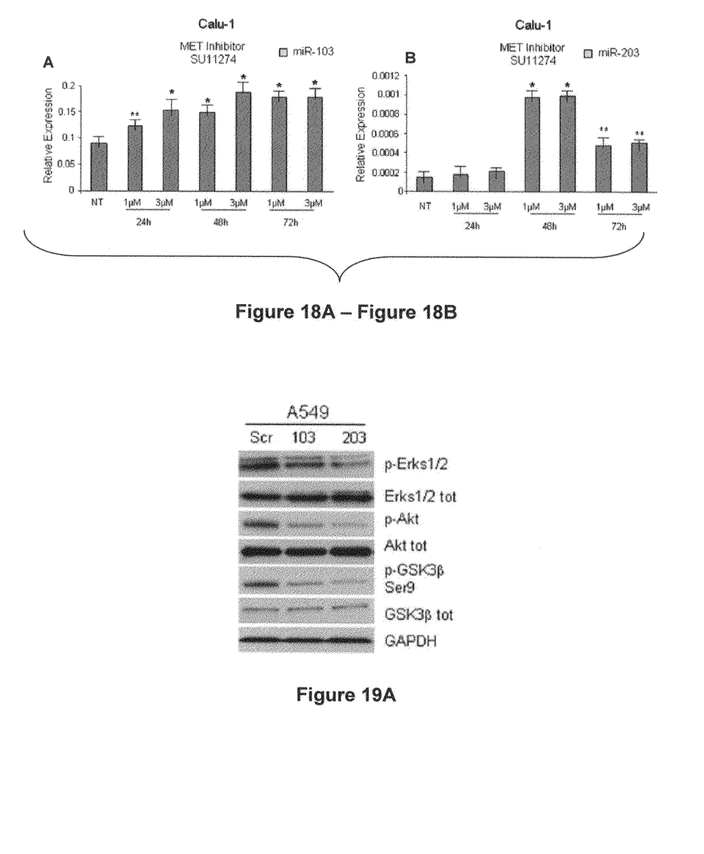

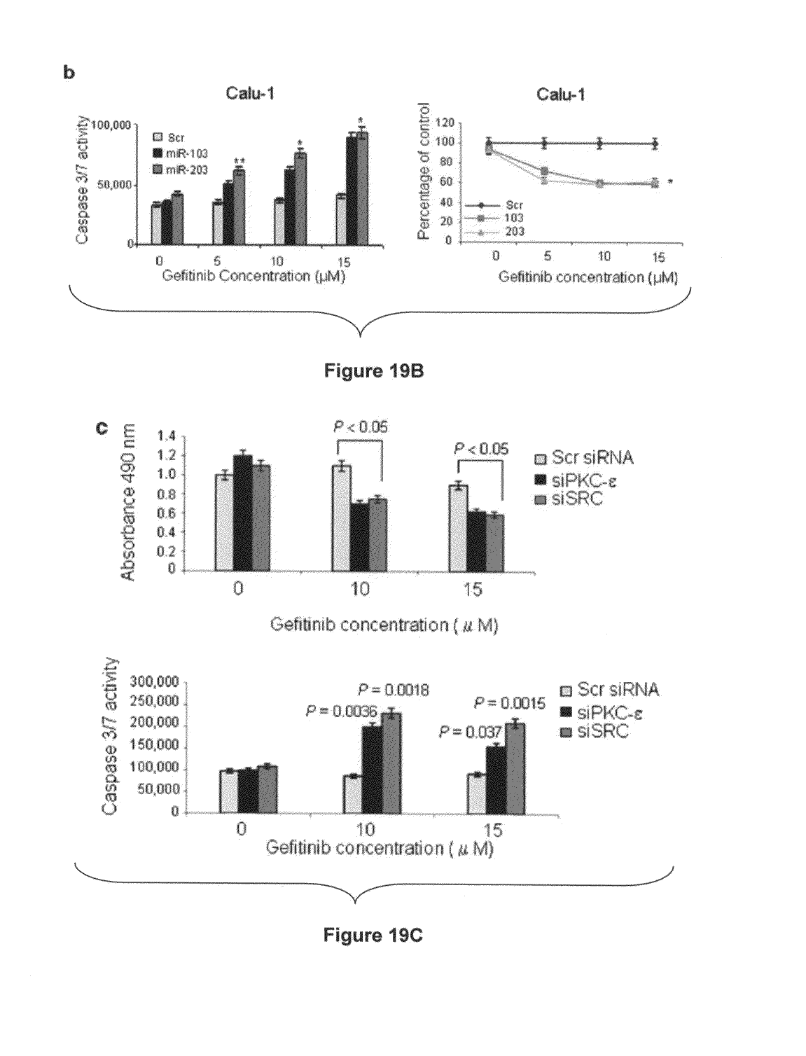

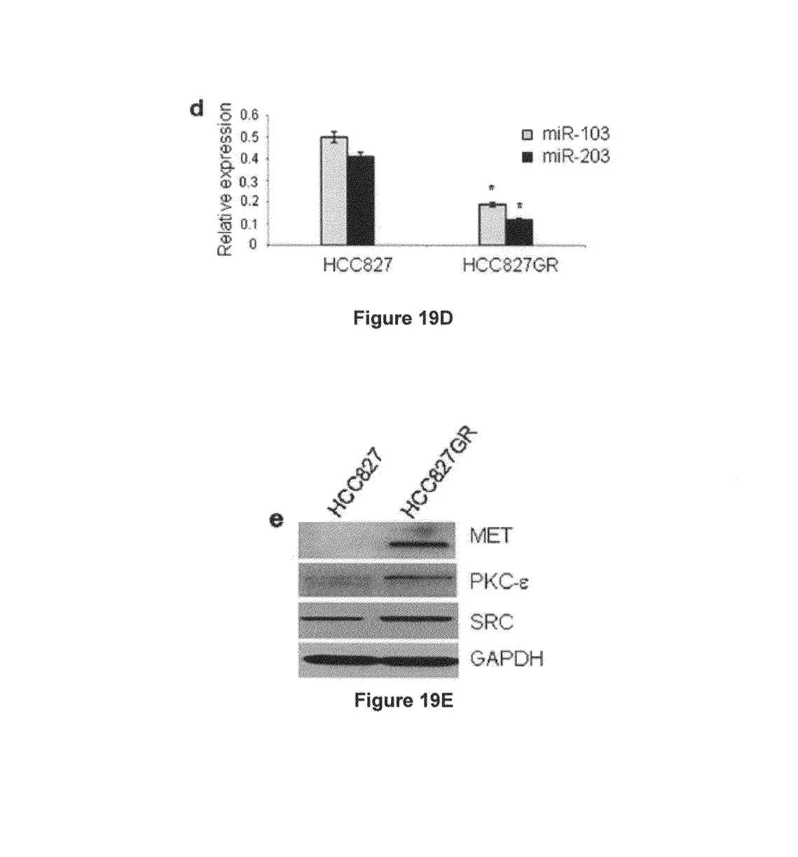

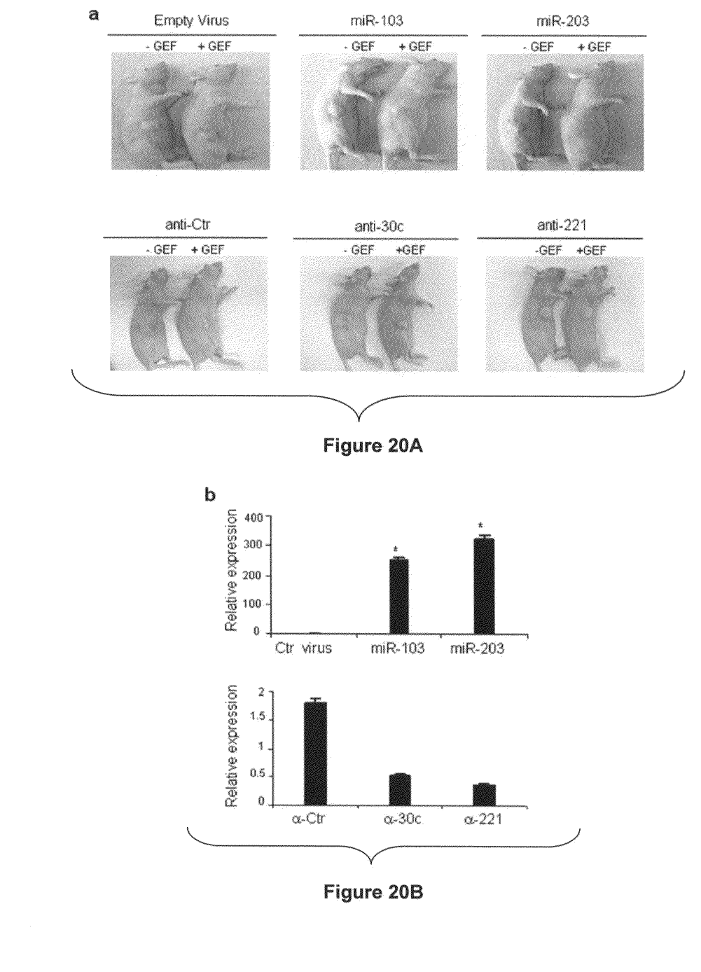

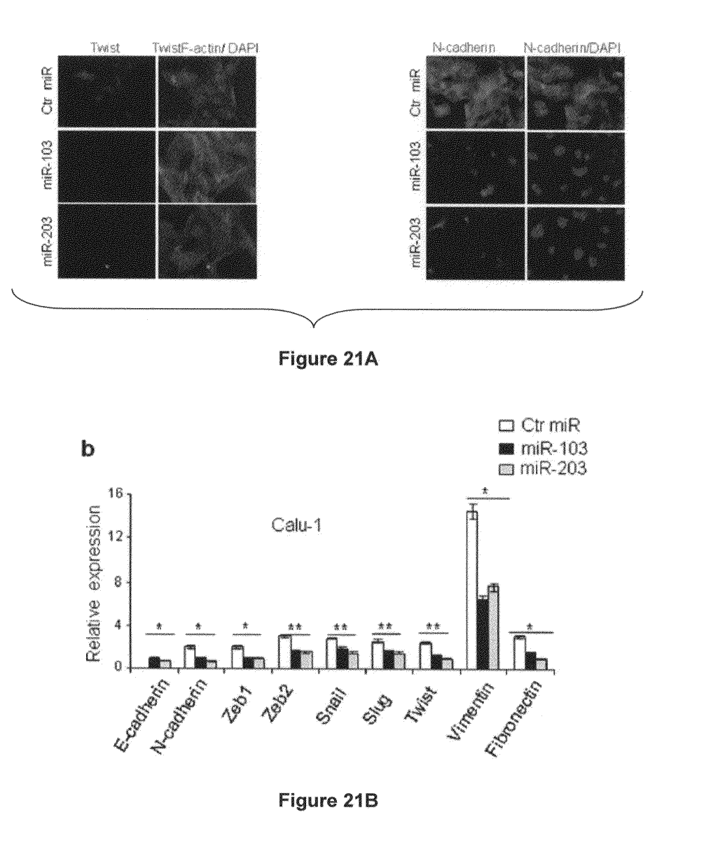

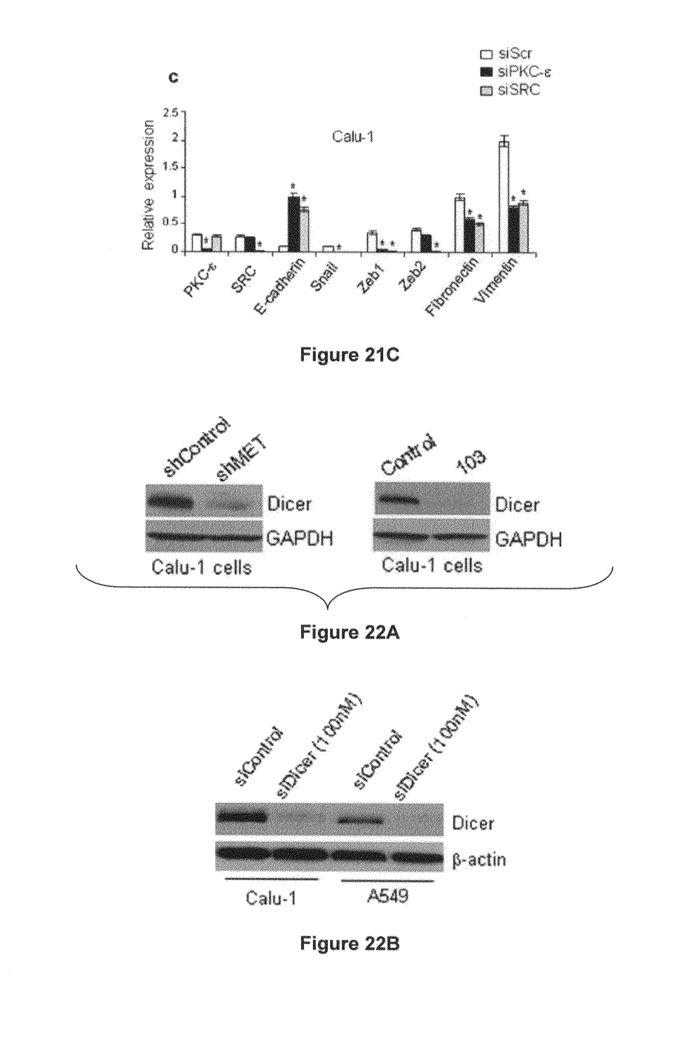

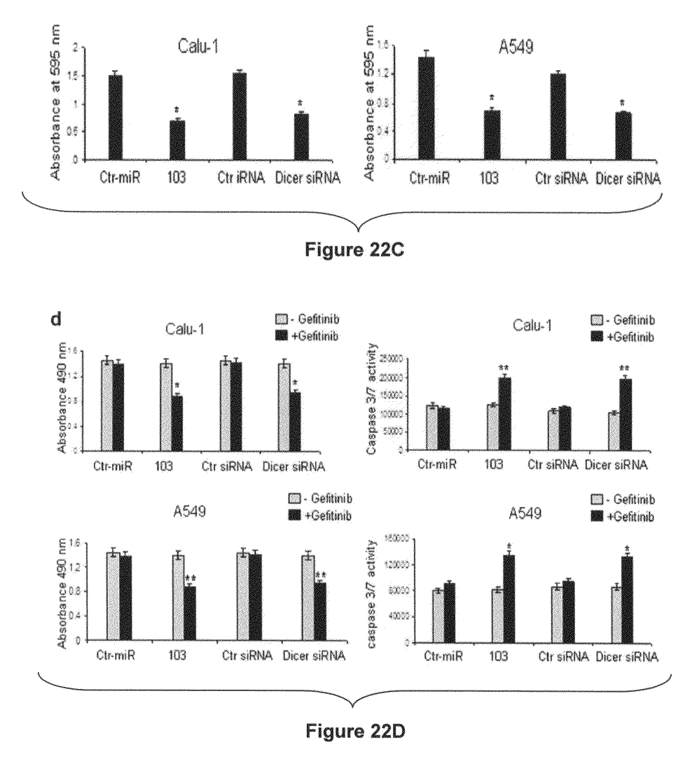

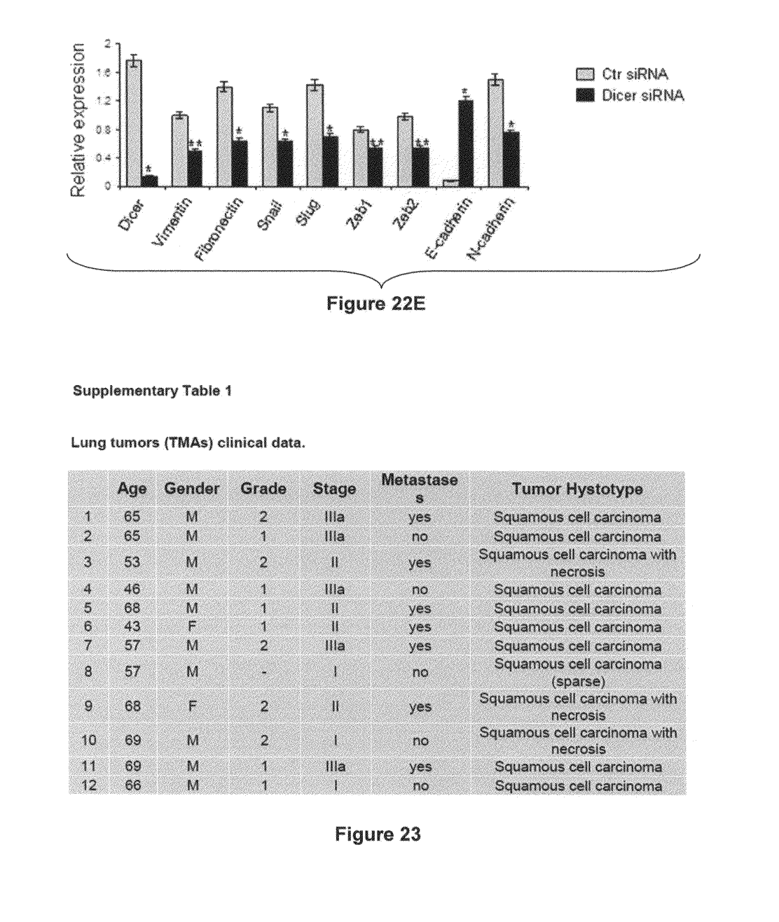

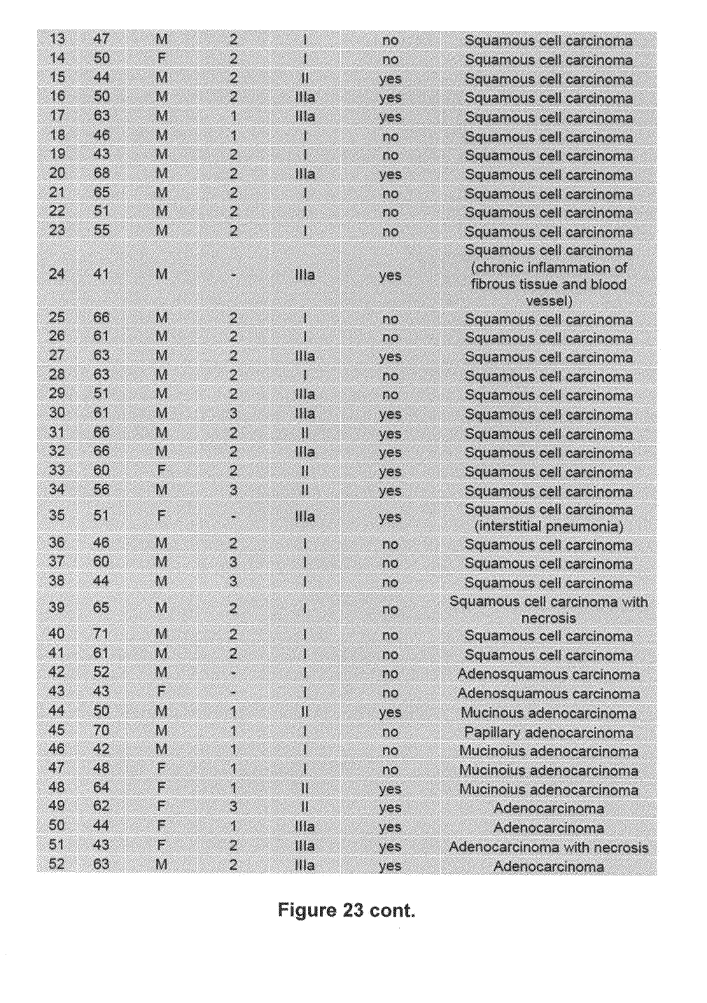

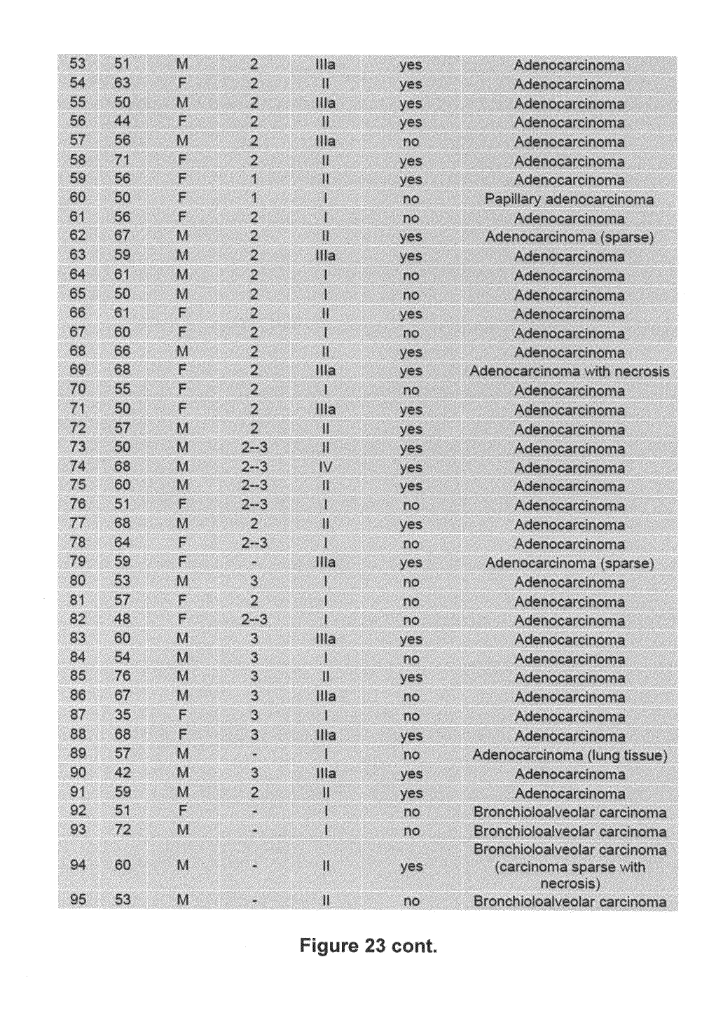

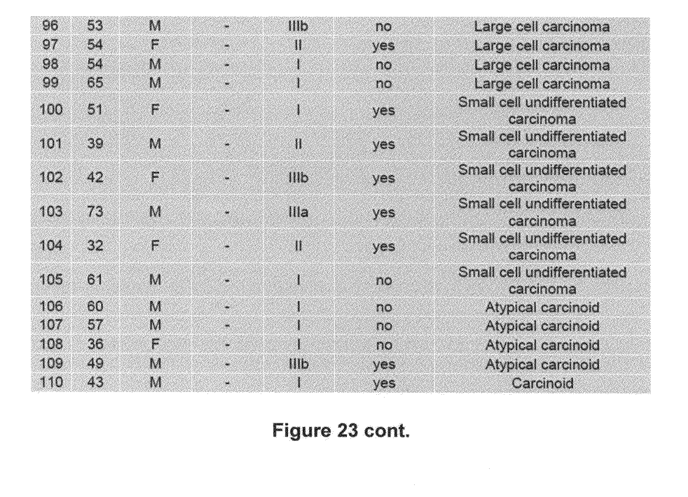

FIGS. 16A-6B. miR-21, miR-29a/c, miR-100 are involved in gefitinib-induced apoptosis. Enforced expression of miR-21, miR-29a/c, miR-100 increases cell viability and reduces caspase 3/7 activity in HCC827 cells (FIG. 16A) and PC9 cells (FIG. 16B) exposed to 10 μM gefitinib for 24 h. One representative of three independent experiments is shown. Relative values are shown as mean and ±s.d. Two tailed student's t test was used to determine P values.

FIGS. 17A-17B. miR-21 knockdown increases gefitinib sensitivity.

FIG. 17A. miR-21 silencing by anti-miR oligonucleotides in A549, HCC827GR and PC9GR cells decreases cell viability as assessed by MTS assay, and FIG. 17B increases cell death, by caspase 3/7 assay, after gefitinib treatment (10 μM) for 24 h. Error bars depict s.d. Results from at least three independent experiments are reported. *P<0.05, **P<0.001 by tow tailed student t test.

FIGS. 18A-18B. MET inhibitor SU11274 induces miR-103 and miR-203 upregulation. Calu-1 cells were exposed to different SU11274 concentrations (1 and 3 μM) 1 for 24, 48 and 72 h. miR-103 and miR-203 expression levels were assessed by qRT-PCR, as described herein. Results are representative of at least three independent experiments. Error bars depict ±s.d. *P<0.001,**P<0.05.

FIG. 19A-19E. PKC-ε and SRC knockdown induces gefitinib sensitivity.

FIG. 19A. miR-103, miR203 inforced expression in A549 cells inhibits AKT/ERKs pathways. β-actin levels were used as loading control. One representative of three independent experiments is shown.

FIG. 19B. miR-103, miR-203 overexpression in Calu-1 cells induces gefitinib sensitivity as assessed by caspase 3/7 and MTT assays. Results are representative of at least four independent experiments.

FIG. 19C. Viability and caspase 3/7 assays in Calu-1 cells after PKC-H and SRC knockdown followed by gefitinib treatment (10 μM, 15 μM) for 24 h.

FIG. 19D. qRT-PCT showing miR-103 and miR-203 deceased expression in HCC827GR cells, with MET amplification, compared to the parental HCC827 cells.

FIG. 19E. Western blot showing increased expression of PKC-ε and SRC in HCC827GR with MET amplification, compared to the parental HCC837 gefitinib-sensitive cells. Experiments were performed three times in triplicate. Error bars represent ±s.d. P values were determined by student's t test. *P<0.005.

FIGS. 20A-20B. miR-103, miR-203, miR-221, miR-30c effects in vivo.

FIG. 20A. Comparison of tumor engraftments in nude mice injected with A549 cells stable infected with Empty virus, miR-103, miR-203 and with anti-Ctr, anti-221, anti-30c. 35 days from the injection and after treatment with vehicle (0.1% tween 80) or gefitinib (200 mg/kg) mice were sacrificed. The images show one mouse from among five of each category.

FIG. 20B. qRT-PCR showing miR-103, miR-203 upregulation and miR-30c, miR-221 downregulation in tumor xenografts. Data are presented as ±s.d. *P<0.001.

FIGS. 21A-21C. miR-103 and miR-203 overexpression induces MET.

FIG. 21A. Immunofluorescence showing Twist and N-cadherin downregulation after miR-103 and miR-203 enforced expression.

FIGS. 21B-21C. qRT-PCRs after miR-103 and miR-203 enforced expression and PKC-ε and SRC silencing in Calu-1 cells. miR-103, miR-203 overexpression and PKC-ε, SRC knockdown induces a decrease in mesenchymal markers and an increase in E-cadherin mRNAs expression levels. Error bars depict s.d. Results from at least three independent experiments are reported. *P<0.001, **P<0.05.

FIGS. 22A-22E. Dicer silencing promotes gefitinib sensitivity and MET in NSCLC.

FIG. 22A. Dicer down-regulation after MET stable knockdown and after miR-103 enforced expression in Calu-1 cells.

FIG. 22B. Dicer downregulation after transfection of Calu-1 and A549 cells with 100 nM of Dicer siRNA.

FIG. 22C. Dicer knockdown reduces cell migration in Calu-1 and A549 cells. Graphs show the absolute number of cells migrating through the transwell quantified by measuring the absorbance at 595 nm.

FIG. 22D. MTS and caspase 3/7 assays showing how Dicer silencing increases sensitivity to gefitinib-induced apoptosis. Results are representative of at least, three independent experiments.

FIG. 22E. qRT-PCR showing that Dicer depletion influences mesenchymal-epithelial transition (MET) by regulating the expression of mesenchymal and epithelial markers. Error bars depict ±s.d. of four independent experiments in c and d. * P<0.005, ** P<0.05.

FIG. 23. Clinical Table 1—Detection of PKC-ε, SRC, APAF-1 and BIM proteins in vivo in 110 lung cancer specimens.

FIG. 24. Clinical Table 2—Forty independent lung tumors with an annotated clinical history.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention in based, at least in part, on research findings that EGF and MET receptors, by modulating specific miRNAs, control gefitinib-induced apoptosis and NSCLC tumorigenesis. Identified herein are EGF- and MET-receptor-regulated miRNAs representing oncogenic signaling networks in NSCLCs.

As used herein interchangeably, a “miR gene product,” “microRNA,” “miR,” or “miRNA” refers to the unprocessed or processed RNA transcript from a miR gene. As the miR gene products are not translated into protein, the term “miR gene products” does not include proteins. The unprocessed miR gene transcript is also called a “miR precursor,” and typically comprises an RNA transcript of about 70-100 nucleotides in length. The miR precursor can be processed by digestion with an RNAse (for example, Dicer, Argonaut, RNAse III (e.g., E. coli RNAse III)) into an active 19-25 nucleotide RNA molecule. This active 19-25 nucleotide RNA molecule is also called the “processed” miR gene transcript or “mature” miRNA.

The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAse III). It is understood that the active 19-25 nucleotide RNA molecule can also be produced directly by biological or chemical synthesis, without having to be processed from the miR precursor. When a microRNA is referred to herein by name, the name corresponds to both the precursor and mature forms, unless otherwise indicated.

As used herein, a “subject” can be any mammal that has, or is suspected of having, cancer. In a preferred embodiment, the subject is a human who has, or is suspected of having, cancer.

The level of at least one miR gene product can be measured in cells of a biological sample obtained from the subject. For example, a tissue sample can be removed from a subject suspected of having cancer, by conventional biopsy techniques. In another embodiment, a blood sample can be removed from the subject, and white blood cells can be isolated for DNA extraction by standard techniques. The blood or tissue sample is preferably obtained from the subject prior to initiation of radiotherapy, chemotherapy or other therapeutic treatment. A corresponding control tissue or blood sample, or a control reference sample, can be obtained from unaffected tissues of the subject, from a normal human individual or population of normal individuals, or from cultured cells corresponding to the majority of cells in the subject's sample. The control tissue or blood sample is then processed along with the sample from the subject, so that the levels of miR gene product produced from a given miR gene in cells from the subject's sample can be compared to the corresponding miR gene product levels from cells of the control sample. Alternatively, a reference sample can be obtained and processed separately (e.g., at a different time) from the test sample and the level of a miR gene product produced from a given miR gene in cells from the test sample can be compared to the corresponding miR gene product level from the reference sample.

The level of a miR gene product in a sample can be measured using any technique that is suitable for detecting RNA expression levels in a biological sample. Suitable techniques (e.g., Northern blot analysis, RT-PCR, in situ hybridization) for determining RNA expression levels in a biological sample (e.g., cells, tissues) are well known to those of skill in the art. In a particular embodiment, the level of at least one miR gene product is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes (e.g., DNA probes, RNA probes) for Northern blot hybridization of a given miR gene product can be produced from the nucleic acid sequences provided herein and include, but are not limited to, probes having at least about 70%, 75%, 80%, 85%, 90%, 95%, 98% or 99% complementarity to a miR gene product of interest, as well as probes that have complete complementarity to a miR gene product of interest. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are incorporated herein by reference.

For example, the nucleic acid probe can be labeled with, e.g., a radionuclide, such as 3H, 32P, 33P, 14C, or 35S; a heavy metal; a ligand capable of functioning as a specific binding pair member for a labeled ligand (e.g., biotin, avidin or an antibody); a fluorescent molecule; a chemiluminescent molecule; an enzyme or the like.

Probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are incorporated herein by reference. The latter is the method of choice for synthesizing 32P-labeled probes of high specific activity from single-stranded DNA or from RNA templates. For example, by replacing preexisting nucleotides with highly radioactive nucleotides according to the nick translation method, it is possible to prepare 32P-labeled nucleic acid probes with a specific activity well in excess of 108 cpm/microgram.

Autoradiographic detection of hybridization can then be performed by exposing hybridized filters to photographic film. Densitometric scanning of the photographic films exposed by the hybridized filters provides an accurate measurement of miR gene transcript levels. Using another approach, miR gene transcript levels can be quantified by computerized imaging systems, such as the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin and antibodies (e.g., anti-biotin antibodies) coupled to fluorescent dyes or enzymes that produce color reactions.

In addition to Northern and other RNA hybridization techniques, determining the levels of RNA transcripts can be accomplished using the technique of in situ hybridization. This technique requires fewer cells than the Northern blotting technique and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given miR gene product can be produced from the nucleic acid sequences provided herein, and include, but are not limited to, probes having at least about 70%, 75%, 80%, 85%, 90%, 95%, 98% or 99% complementarity to a miR gene product of interest, as well as probes that have complete complementarity to a miR gene product of interest, as described above.

The relative number of miR gene transcripts in cells can also be determined by reverse transcription of miR gene transcripts, followed by amplification of the reverse-transcribed transcripts by polymerase chain reaction (RT-PCR). The levels of miR gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). Methods for performing quantitative and semi-quantitative RT-PCR, and variations thereof, are well known to those of skill in the art.

In some instances, it may be desirable to simultaneously determine the expression level of a plurality of different miR gene products in a sample. In other instances, it may be desirable to determine the expression level of the transcripts of all known miR genes correlated with a cancer. Assessing cancer-specific expression levels for hundreds of miR genes or gene products is time consuming and requires a large amount of total RNA (e.g., at least 20 μg for each Northern blot) and autoradiographic techniques that require radioactive isotopes.

To overcome these limitations, an oligolibrary, in microchip format (i.e., a microarray), may be constructed containing a set of oligonucleotide (e.g., oligodeoxynucleotide) probes that are specific for a set of miR genes. Using such a microarray, the expression level of multiple microRNAs in a biological sample can be determined by reverse transcribing the RNAs to generate a set of target oligodeoxynucleotides, and hybridizing them to probe the oligonucleotides on the microarray to generate a hybridization, or expression, profile. The hybridization profile of the test sample can then be compared to that of a control sample to determine which microRNAs have an altered expression level in lung cancer metastasis and/or recurrence cells. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “miR-specific probe oligonucleotide” or “probe oligonucleotide specific for a miR” is meant a probe oligonucleotide that has a sequence selected to hybridize to a specific miR gene product, or to a reverse transcript of the specific miR gene product.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal tissue may be distinguished from cancer cells, and within cancer cell types, different prognosis states (for example, good or poor long term survival prospects) may be determined. By comparing expression profiles of cells in different states, information regarding which genes are important (including both up- and down-regulation of genes) in each of these states is obtained. The identification of sequences that are differentially expressed in cancer cells or normal cells, as well as differential expression resulting in different prognostic outcomes, allows the use of this information in a number of ways. For example, a particular treatment regime may be evaluated (e.g., to determine whether a chemotherapeutic drug acts to improve the long-term prognosis in a particular patient). Similarly, diagnosis may be done or confirmed by comparing patient samples with known expression profiles. Furthermore, these gene expression profiles (or individual genes) allow screening of drug candidates that suppress the miR or disease expression profile or convert a poor prognosis profile to a better prognosis profile.

A microarray can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs and other RNAs (e.g., rRNAs, mRNAs) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length, e.g., 40 nucleotides, are 5′-amine modified at position C6 and printed using commercially available microarray systems, e.g., the GeneMachine OmniGrid™ 100 Microarrayer and Amersham CodeLink™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRs, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding miR in the patient sample.

The use of the array has several advantages for miRNA expression detection. First, the global expression of several hundred genes can be identified in the same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) allows the construction of a common microarray for several species, with distinct oligonucleotide probes for each. Such a tool would allow for analysis of trans-species expression for each known miR under various conditions.

In addition to use for quantitative expression level assays of specific miRs, a microchip containing miRNA-specific probe oligonucleotides corresponding to a substantial portion of the miRNome, preferably the entire miRNome, may be employed to carry out miR gene expression profiling, for analysis of miR expression patterns. Distinct miR signatures can be associated with established disease markers, or directly with a disease state.

According to the expression profiling methods described herein, total RNA from a sample from a subject suspected of having a cancer profile (eg. metastasis or recurrence) is quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal, e.g., noncancerous, control sample. The signal is indicative of the presence of, or propensity to develop, the cancer profile in the subject.

Other techniques for measuring miR gene expression are also within the skill in the art, and include various techniques for measuring rates of RNA transcription and degradation.

The invention also provides methods of determining the prognosis. Examples of an adverse prognosis include, but are not limited to, low survival rate and rapid disease progression.

In certain embodiments, the level of the at least one miR gene product is measured by reverse transcribing RNA from a test sample obtained from the subject to provide a set of target oligodeoxynucleotides, hybridizing the target oligodeoxynucleotides to a microarray that comprises miRNA-specific probe oligonucleotides to provide a hybridization profile for the test sample, and comparing the test sample hybridization profile to a hybridization profile generated from a control sample.

Accordingly, the present invention encompasses methods of treating cancer in a subject. The method comprises administering an effective amount of the at least one isolated antisense miR gene product, or an isolated variant or biologically-active fragment thereof, such that metastasis, recurrence or proliferation of cancer cells in the subject is inhibited. The isolated antisense miR gene product that is administered to the subject can be complementary to an identical to an endogenous wild-type miR gene product or it can be complementary a variant or biologically-active fragment thereof.

As defined herein, a “variant” of a miR gene product refers to a miRNA that has less than 100% identity to a corresponding wild-type miR gene product and possesses one or more biological activities of the corresponding wild-type miR gene product. Examples of such biological activities include, but are not limited to, inhibition of a cellular process associated with lung metastasis or recurrence (e.g., cell differentiation, cell growth, cell death). These variants include species variants and variants that are the consequence of one or more mutations (e.g., a substitution, a deletion, an insertion) in a miR gene. In certain embodiments, the variant is at least about 70%, 75%, 80%, 85%, 90%, 95%, 98%, or 99% identical to a corresponding wild-type miR gene product.

As defined herein, a “biologically-active fragment” of a miR gene product refers to an RNA fragment of a miR gene product that possesses one or more biological activities of a corresponding wild-type miR gene product. As described above, examples of such biological activities include, but are not limited to, inhibition of a cellular process associated with lung cancer metastasis or recurrence. In certain embodiments, the biologically-active fragment is at least about 5, 7, 10, 12, 15, or 17 nucleotides in length. In a particular embodiment, an isolated miR gene product can be administered to a subject in combination with one or more additional anti-cancer treatments. Suitable anti-cancer treatments include, but are not limited to, chemotherapy, radiation therapy and combinations thereof (e.g., chemoradiation).

The terms “treat”, “treating” and “treatment”, as used herein, refer to ameliorating symptoms associated with a disease or condition, for example, lung cancer metastasis and/or recurrence, including preventing or delaying the onset of the disease symptoms, and/or lessening the severity or frequency of symptoms of the disease or condition. The terms “subject” and “individual” are defined herein to include animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “effective amount” of an isolated miR gene product is an amount sufficient to inhibit proliferation of a cancer cell in a subject suffering from lung cancer metastasis and/or recurrence. One skilled in the art can readily determine an effective amount of a miR gene product to be administered to a given subject, by taking into account factors, such as the size and weight of the subject; the extent of disease penetration; the age, health and sex of the subject; the route of administration; and whether the administration is regional or systemic.

For example, an effective amount of an isolated miR gene product can be based on the approximate weight of a tumor mass to be treated. The approximate weight of a tumor mass can be determined by calculating the approximate volume of the mass, wherein one cubic centimeter of volume is roughly equivalent to one gram. An effective amount of the isolated miR gene product based on the weight of a tumor mass can be in the range of about 10-500 micrograms/gram of tumor mass. In certain embodiments, the tumor mass can be at least about 10 micrograms/gram of tumor mass, at least about 60 micrograms/gram of tumor mass or at least about 100 micrograms/gram of tumor mass.

An effective amount of an isolated miR gene product can also be based on the approximate or estimated body weight of a subject to be treated. Preferably, such effective amounts are administered parenterally or enterally, as described herein. For example, an effective amount of the isolated miR gene product that is administered to a subject can range from about 5-3000 micrograms/kg of body weight, from about 700-1000 micrograms/kg of body weight, or greater than about 1000 micrograms/kg of body weight.

One skilled in the art can also readily determine an appropriate dosage regimen for the administration of an isolated miR gene product to a given subject. For example, a miR gene product can be administered to the subject once (e.g., as a single injection or deposition). Alternatively, a miR gene product can be administered once or twice daily to a subject for a period of from about three to about twenty-eight days, more particularly from about seven to about ten days. In a particular dosage regimen, a miR gene product is administered once a day for seven days. Where a dosage regimen comprises multiple administrations, it is understood that the effective amount of the miR gene product administered to the subject can comprise the total amount of gene product administered over the entire dosage regimen.

As used herein, an “isolated” miR gene product is one that is synthesized, or altered or removed from the natural state through human intervention. For example, a synthetic miR gene product, or a miR gene product partially or completely separated from the coexisting materials of its natural state, is considered to be “isolated.” An isolated miR gene product can exist in a substantially-purified form, or can exist in a cell into which the miR gene product has been delivered. Thus, a miR gene product that is deliberately delivered to, or expressed in, a cell is considered an “isolated” miR gene product. A miR gene product produced inside a cell from a miR precursor molecule is also considered to be an “isolated” molecule. According to the invention, the isolated miR gene products described herein can be used for the manufacture of a medicament for treating lung cancer metastasis and/or recurrence in a subject (e.g., a human).

Isolated miR gene products can be obtained using a number of standard techniques. For example, the miR gene products can be chemically synthesized or recombinantly produced using methods known in the art. In one embodiment, miR gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the miR gene products can be expressed from recombinant circular or linear DNA plasmids using any suitable promoter. Suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the miR gene products in cancer cells.

The miR gene products that are expressed from recombinant plasmids can be isolated from cultured cell expression systems by standard techniques. The miR gene products that are expressed from recombinant plasmids can also be delivered to, and expressed directly in, the cancer cells. The use of recombinant plasmids to deliver the miR gene products to cancer cells is discussed in more detail below.

The miR gene products can be expressed from a separate recombinant plasmid, or they can be expressed from the same recombinant plasmid. In one embodiment, the miR gene products are expressed as RNA precursor molecules from a single plasmid, and the precursor molecules are processed into the functional miR gene product by a suitable processing system, including, but not limited to, processing systems extant within a cancer cell. Other suitable processing systems include, e.g., the in vitro Drosophila cell lysate system (e.g., as described in U.S. Published Patent Application No. 2002/0086356 to Tuschl et al., the entire disclosure of which is incorporated herein by reference) and the E. coli RNAse III system (e.g., as described in U.S. Published Patent Application No. 2004/0014113 to Yang et al., the entire disclosure of which is incorporated herein by reference).

Selection of plasmids suitable for expressing the miR gene products, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are incorporated herein by reference.

In one embodiment, a plasmid expressing the miR gene products comprises a sequence encoding a miR precursor RNA under the control of the CMV intermediate-early promoter. As used herein, “under the control” of a promoter means that the nucleic acid sequences encoding the miR gene product are located 3′ of the promoter, so that the promoter can initiate transcription of the miR gene product coding sequences.

The miR gene products can also be expressed from recombinant viral vectors. It is contemplated that the miR gene products can be expressed from two separate recombinant viral vectors, or from the same viral vector. The RNA expressed from the recombinant viral vectors can either be isolated from cultured cell expression systems by standard techniques, or can be expressed directly in cancer cells. The use of recombinant viral vectors to deliver the miR gene products to cancer cells is discussed in more detail below.

The recombinant viral vectors of the invention comprise sequences encoding the miR gene products and any suitable promoter for expressing the RNA sequences. Suitable promoters include, but are not limited to, the U6 or H1 RNA pol III promoter sequences, or the cytomegalovirus promoters. Selection of other suitable promoters is within the skill in the art. The recombinant viral vectors of the invention can also comprise inducible or regulatable promoters for expression of the miR gene products in a cancer cell.

Any viral vector capable of accepting the coding sequences for the miR gene products can be used; for example, vectors derived from adenovirus (AV); adeno-associated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors that express different capsid protein serotypes are within the skill in the art; see, e.g., Rabinowitz, J. E., et al. (2002), J. Virol. 76:791-801, the entire disclosure of which is incorporated herein by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are incorporated herein by reference.

Particularly suitable viral vectors are those derived from AV and AAV. A suitable AV vector for expressing the miR gene products, a method for constructing the recombinant AV vector, and a method for delivering the vector into target cells, are described in Xia et al. (2002), Nat. Biotech. 20:1006-1010, the entire disclosure of which is incorporated herein by reference. Suitable AAV vectors for expressing the miR gene products, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are incorporated herein by reference. In one embodiment, the miR gene products are expressed from a single recombinant AAV vector comprising the CMV intermediate early promoter.

In a certain embodiment, a recombinant AAV viral vector of the invention comprises a nucleic acid sequence encoding a miR precursor RNA in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the miR sequences from the vector, the polyT termination signals act to terminate transcription.

The number of cancer cells in the body of a subject can be determined by direct measurement, or by estimation from the size of primary or metastatic tumor masses. For example, the number of cancer cells in a subject can be measured by immunohistological methods, flow cytometry, or other techniques designed to detect characteristic surface markers of cancer cells.

A miR gene product can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Particularly suitable administration routes are injection, infusion and direct injection into the tumor.

In the present methods, a miR gene product can be administered to the subject either as naked RNA, in combination with a delivery reagent, or as a nucleic acid (e.g., a recombinant plasmid or viral vector) comprising sequences that express the miR gene product or miR gene expression-inhibiting compound. Suitable delivery reagents include, e.g., the Minis Transit TKO lipophilic reagent; LIPOFECTIN; lipofectamine; cellfectin; polycations (e.g., polylysine) and liposomes.

Recombinant plasmids and viral vectors comprising sequences that express the miR gene products and techniques for delivering such plasmids and vectors to cancer cells, are discussed herein and/or are well known in the art.

In a particular embodiment, liposomes are used to deliver a miR gene product (or nucleic acids comprising sequences encoding them) to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of factors, such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are incorporated herein by reference.

The liposomes for use in the present methods can comprise a ligand molecule that targets the liposome to cancer cells. Ligands that bind to receptors prevalent in cancer cells, such as monoclonal antibodies that bind to tumor cell antigens, are preferred.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both an opsonization-inhibition moiety and a ligand.

Opsonization-inhibiting moieties for use in preparing the liposomes of the invention are typically large hydrophilic polymers that are bound to the liposome membrane. As used herein, an opsonization-inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is incorporated herein by reference.

Opsonization-inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) or derivatives thereof; e.g., methoxy PEG or PPG, and PEG or PPG stearate; synthetic polymers, such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization-inhibiting polymer can be a block copolymer of PEG and either a polyamino acid, polysaccharide, polyamidoamine, polyethyleneamine, or polynucleotide. The opsonization-inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or a derivative thereof. Liposomes modified with PEG or PEG-derivatives are sometimes called “PEGylated liposomes.”

The opsonization-inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH3 and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example, solid tumors (e.g., lung cancer metastasis and/or recurrences), will efficiently accumulate these liposomes; see Gabizon, et al. (1988), Proc. Natl. Acad. Sci., U.S.A., 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the miR gene products (or nucleic acids comprising sequences encoding them) to tumor cells.

The miR gene products can be formulated as pharmaceutical compositions, sometimes called “medicaments,” prior to administering them to a subject, according to techniques known in the art. Accordingly, the invention encompasses pharmaceutical compositions for treating lung cancer metastasis and/or recurrence. In one embodiment, the pharmaceutical composition comprises at least one isolated miR gene product, or an isolated variant or biologically-active fragment thereof, and a pharmaceutically-acceptable carrier. In a particular embodiment, the at least one miR gene product corresponds to a miR gene product that has a decreased level of expression in cancer cells relative to suitable control cells.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

MiRNAs Modulated by Both EGFR and MET

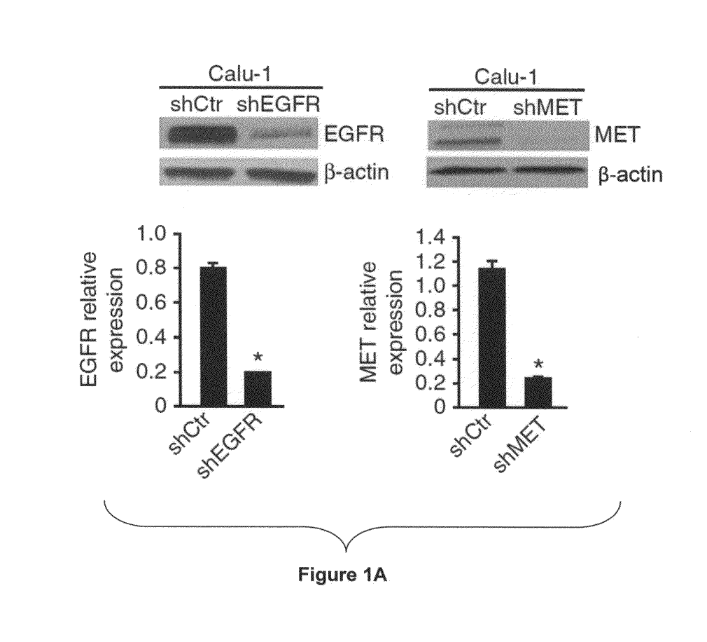

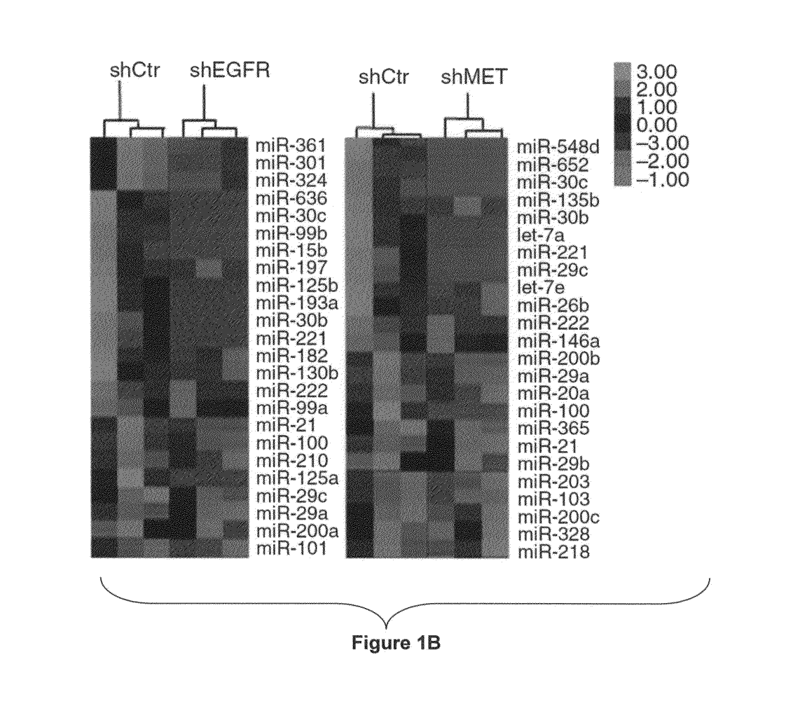

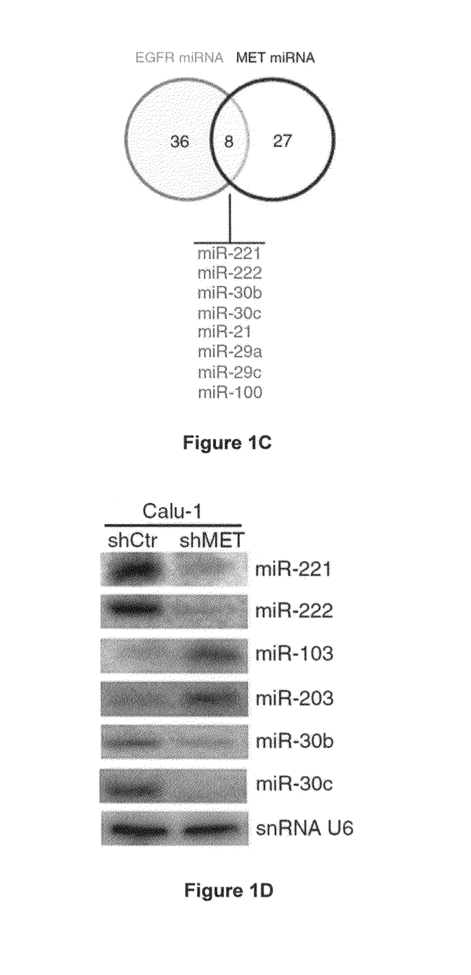

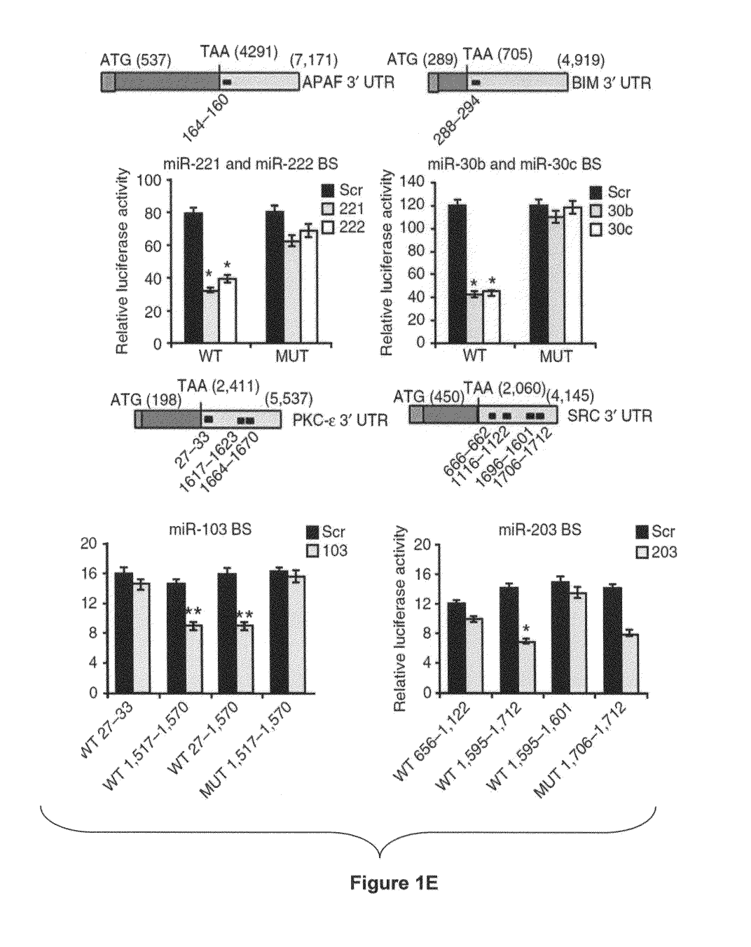

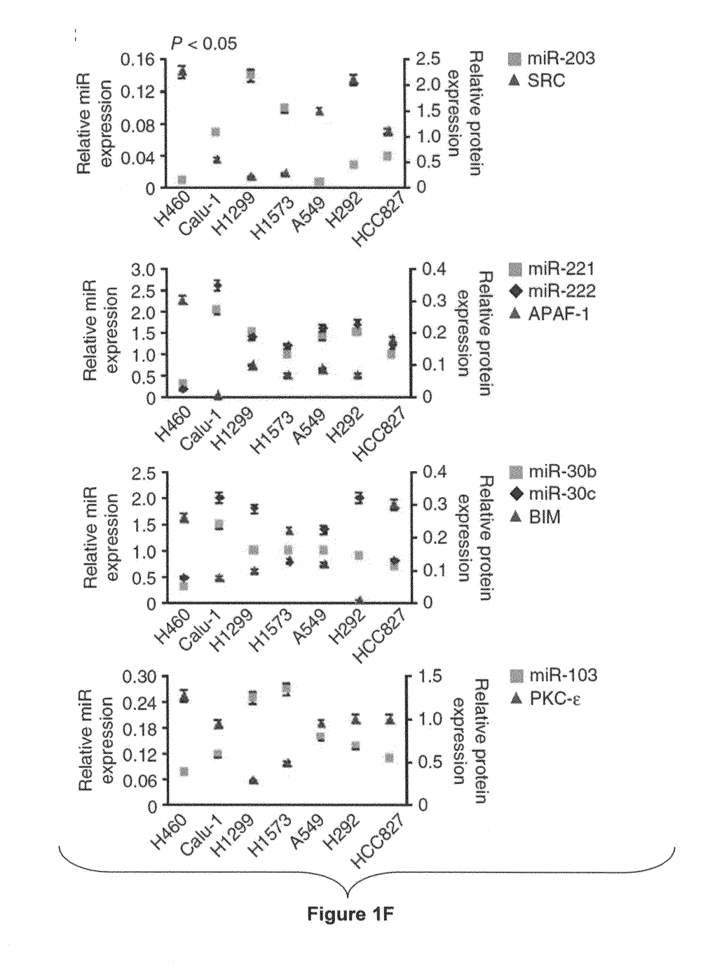

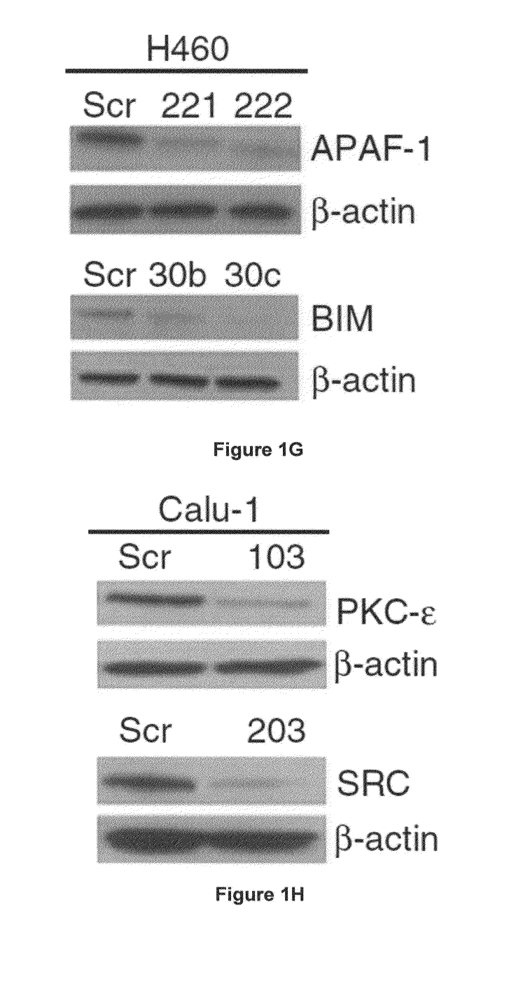

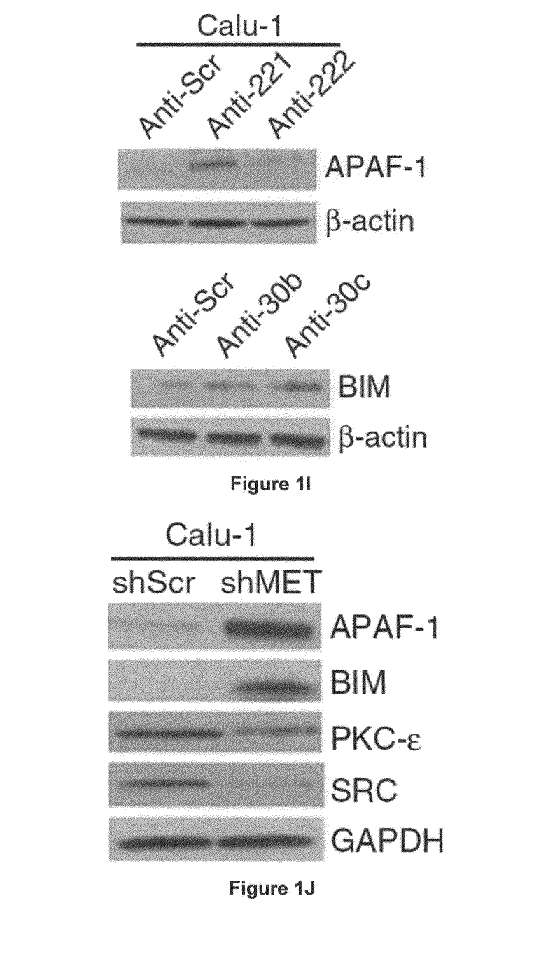

To identify EGFR- and MET-regulated miRNAs, we stably silenced EGFR and MET in Calu-1 cells from the American Type Culture Collection (ATCCC) using shRNA lentiviral particles (FIG. 1A) and examined the global miRNA expression profiles. In EGFR- and MET-knockdown (EGFR-KD and MET-KD) Calu-1 cells, we identified 35 and 44 significantly (P<0.05) dysregulated miRNAs, respectively (FIG. 1B and FIG. 7A).

MiRNAs with a greater than 1.5-fold (for EGFR) or a greater than 1.7-fold (for MET) change are shown. After comparing these two lists of miRNAs, it was found only eight that were regulated by both EGFR and MET (FIG. 1C): miR-21, miR-221 and miR-222, miR-30b and miR-30c, miR-29a and miR-29c and miR-100.

miR-30b, miR-30c, miR-221 and miR-222 were downregulated after both MET and EGFR silencing, and showed the highest fold changes in expression. Also investigated were the two miRNAs that were most differentially induced after MET silencing, miR-103 and miR-203, based on evidence indicating MET overexpression in de novo and acquired resistance to TKIs. The expression of these six miRNAs in EGFR-KD and MET-KD Calu-1 cells were evaluated using quantitative RT-PCR (qRT-PCR) (FIG. 7B) and northern blot (FIG. 1D) analyses.

Tyrosine-Kinase-Modulated miRNA Targets

MET and EGFR RTKs have a key role in lung cancer tumorigenesis and progression. Research analysis shows that miR-103 and miR-203 (which are increased after MET knockdown) are tumor suppressors and that miR-221, miR-222, miR-30b and miR-30c (which are decreased after MET and EGFR silencing) are oncogenic.

The 3′ untranslated regions (3′ UTRs) of human APAF1, BCL2L11 (also known as BIM), PRKCE (also known as PKC-ε) and SRC contain evolutionarily conserved binding sites specific for miR-221 and miR-222, miR-30b and miR-30c, miR-103 and miR-203, respectively (FIG. 8A). These genes were investigated based, in part, on their role in TKI sensitivity (BCL2L11 (BIM) and AP³ or TKI resistance (SRC) or in the negative allosteric modulation of EGFR signaling (PRKCE (PKC-ε)). To determine whether the miRNAs directly interact with these four putative target genes, we cotransfected pGL3 3′ UTR luciferase reporter vectors with synthetic miR-103, miR-203, miR-221, miR-222, miR-30b and miR-30c.

A decrease in luciferase activity indicated direct interactions between the miRNAs and the PRKCE (PKC-ε), SRC, APAF1 and BCL2L11 (BIM) 3′ UTRs (FIG. 1E), and target gene repression was rescued by mutations or deletions in the complementary seed sites (FIG. 1E and FIG. 8A).

A western blot analysis showed an inverse correlation (P<0.05) between miR-221, miR222, miR-103, miR-203, miR-30b and miR-30c expression and the amount of target protein in an NSCLC cell panel (FIGS. 8B, 8C), which was confirmed by determining the Pearson correlation coefficients (FIG. 1F and FIG. 8D).

Results from the immunoblot analysis fully agreed with data obtained using reporter gene assays. Ectopic expression of miR-221, miR-222, miR-30b and miR-30c in H460 cells markedly decreased BIM and APAF-1 expression, and enforced expression of miR-103 and miR-203 clearly reduced the concentrations of PKC-ε and SRC protein (FIG. 1G, FIG. 1H). Conversely, knockdown of miR-221, miR-222, miR-30b and miR-30c increased the concentrations of APAF-1 and BIM protein (FIG. 1-I). As MET knockdown Calu-1 cells showed an increase of APAF-1 and BIM concentrations and a decrease of PKC-ε and SRC concentrations (FIG. 1J), enforced expression of miR-221, miR-222, miR-30b and miR-30c in MET knockdown Calu-1 cells strongly reduced APAF-1 and BIM expression (FIG. 9A), whereas miR-103 and miR-203 knockdown increased SRC and PKC-ε expression (FIG. 9B).

Collectively, these data show a direct correlation between change in expression of PKC-ε, SRC, APAF-1 and BIM proteins and these specific miRNAs after MET silencing in NSCLC cells (FIGS. 1G-1J and FIGS. 9A, 9B). Detection of PKC-ε, SRC, APAF-1 and BIM proteins in vivo in 110 lung cancer specimens (FIG. 23—Clinical Table 1) using miRNA in situ hybridization (ISH) followed by immunohistochemistry (IHC) showed a more significant negative correlation between these proteins and miR-103, miR-203, miR-221, miR-222, miR-30b and miR-30c in human tumors (FIG. 10B).

There was an inverse correlation between miR-203 and SRC expression, miR-30c and BIM expression, miR-103 and PKC-ε expression and miR-222 and APAF-1 expression in the majority of the lung cancer tissues (FIG. 10A, 10B).

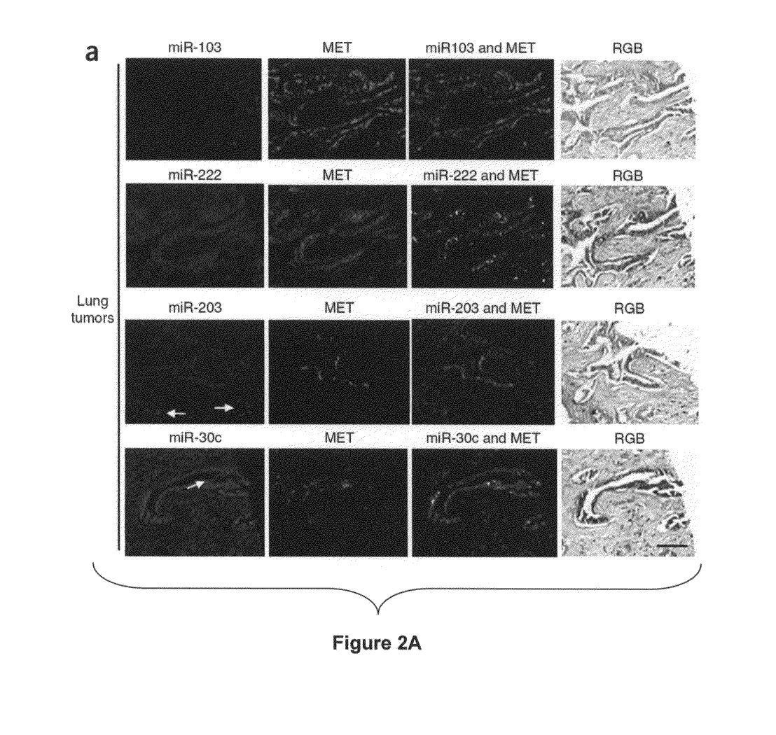

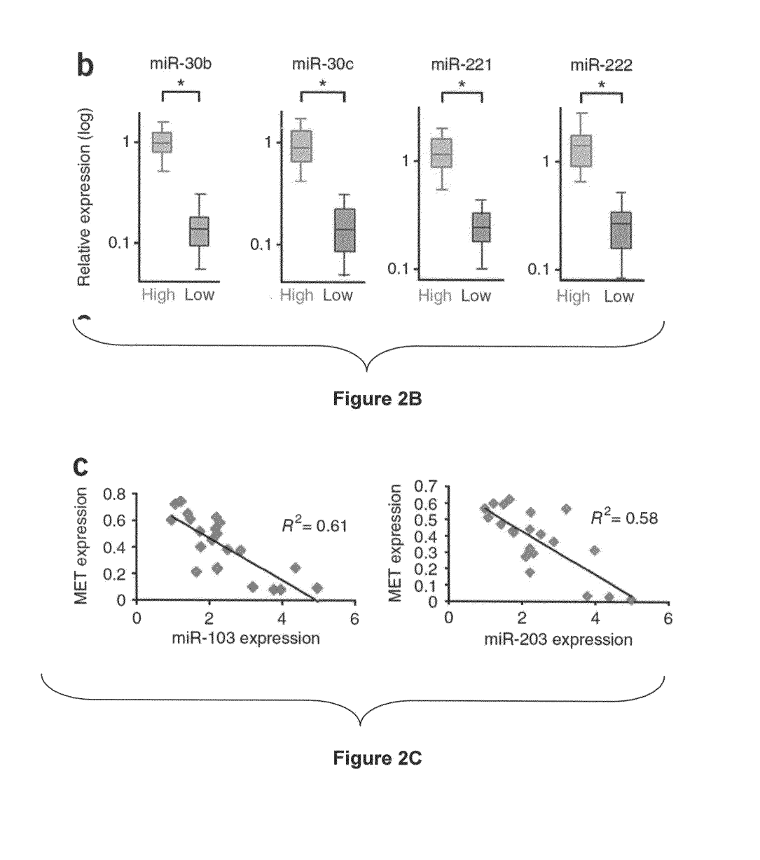

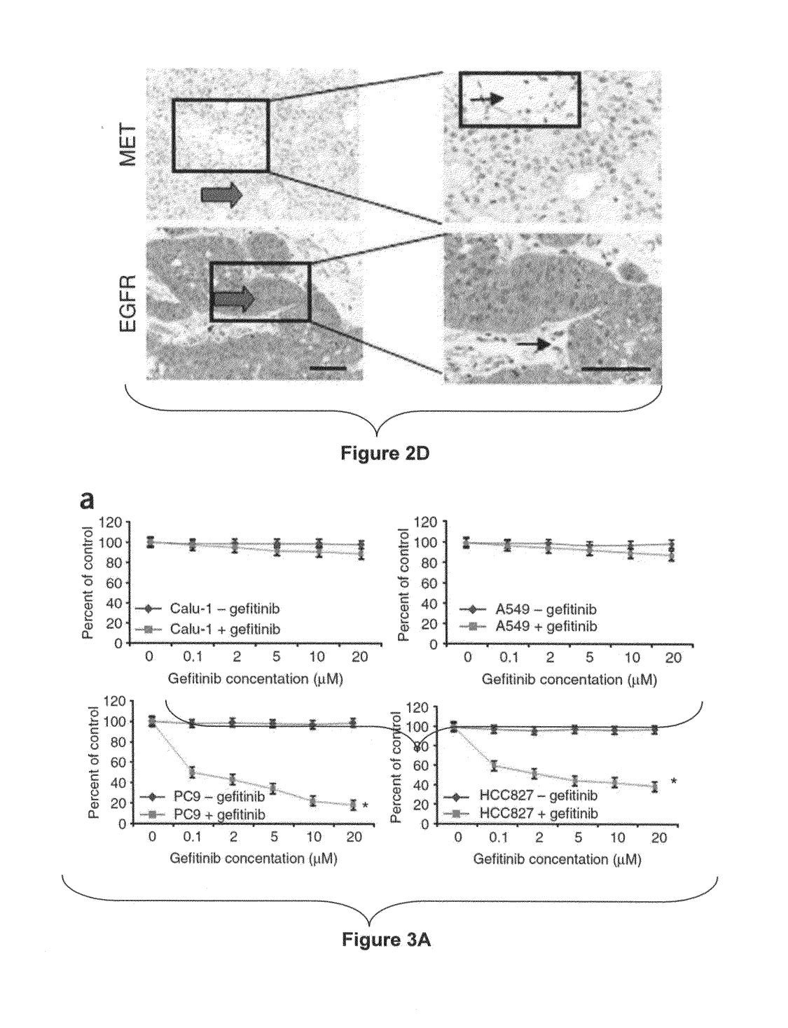

In addition, there was MET overexpression in 52% (57/110) of the same 110 lung tumor samples (shown using miRNA ISH and MET IHC; FIG. 11A), and there was low miR-103 and miR-203 expression and high miR-222 and miR-30c expression in tumors overexpressing MET (FIG. 2A and FIG. 11A); conversely, there was high miR-103 and miR-203 and low miR-222 and miR-30c expression in tumors without MET expression. Notably, the majority of tumors overexpressing MET had accompanying metastases (FIG. 11B), showing that MET-regulated miRNAs have a role in the metastatic spread of lung cancer cells.

The analysis was extended to 40 independent lung tumors with an annotated clinical history (FIG. 24—Clinical Table 2), which were divided into two groups of ‘low’ and ‘high’ MET and EGFR expression based on qRT-PCR analyses (FIG. 2 and FIG. 11C).

An analysis of variance confirmed that the miRNAs (miR-30b and miR-30c and miR-221 and miR-222) were differentially expressed between the low and high groups, whereas using a Pearson coefficient, an inverse correlation was identified between MET and miR-103 and MET and miR-203 (FIGS. 2B, 2C).

The qRT-PCR results were confirmed using an IHC analysis for MET and EGFR (FIG. 11D). In addition, MET overexpression was observed in tumors that had distant metastases compared to non-metastatic tumors, but there was no correlation between metastases and EGFR expression in these 40 lung cancers (FIG. 2D and FIG. 11E).

Tyrosine-Kinase—Regulated miRNAs Control Gefitinib Sensitivity

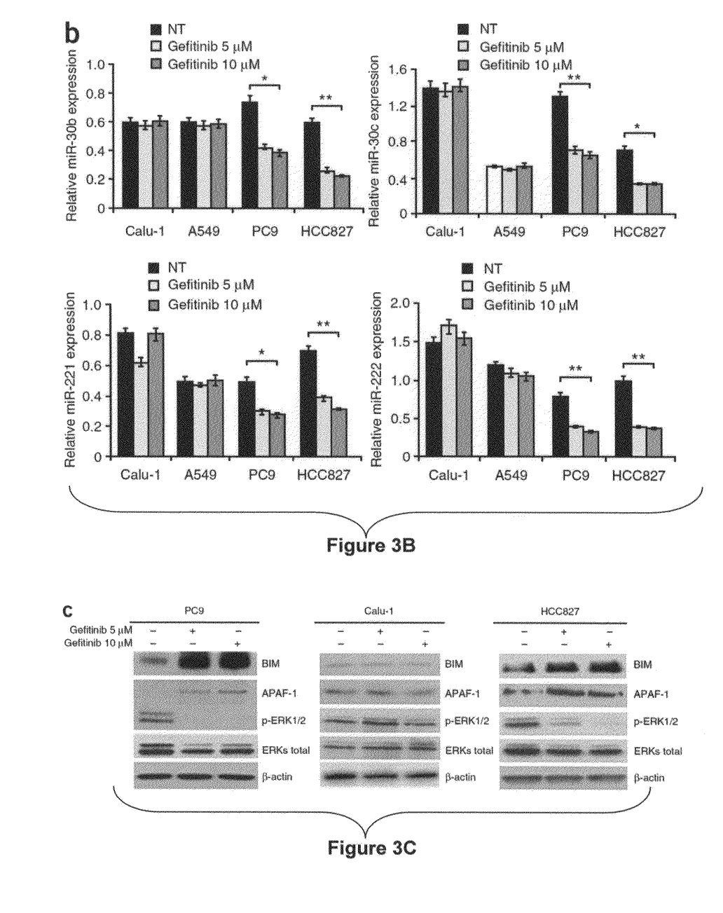

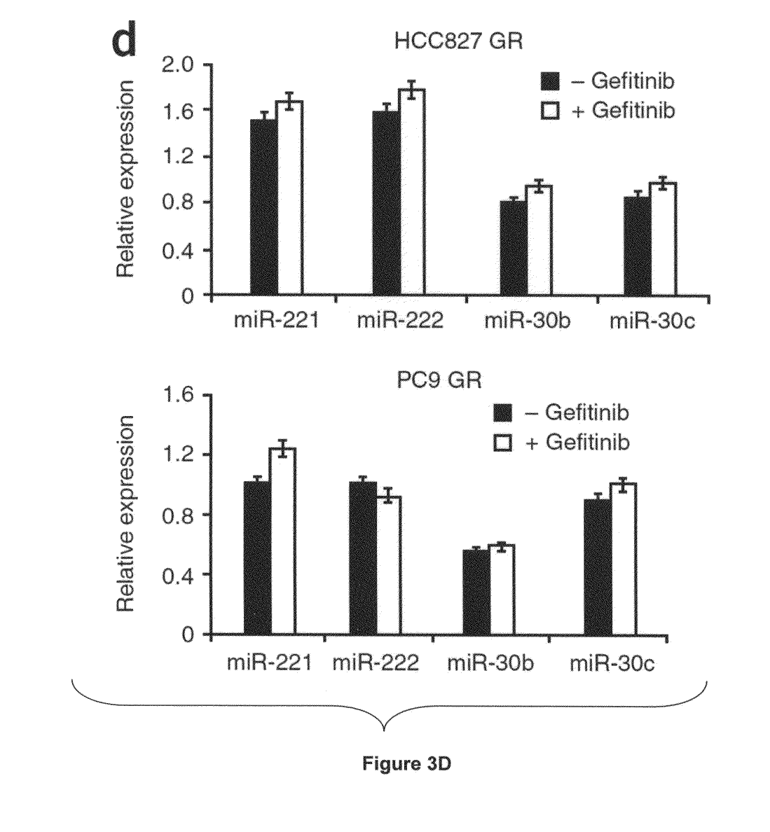

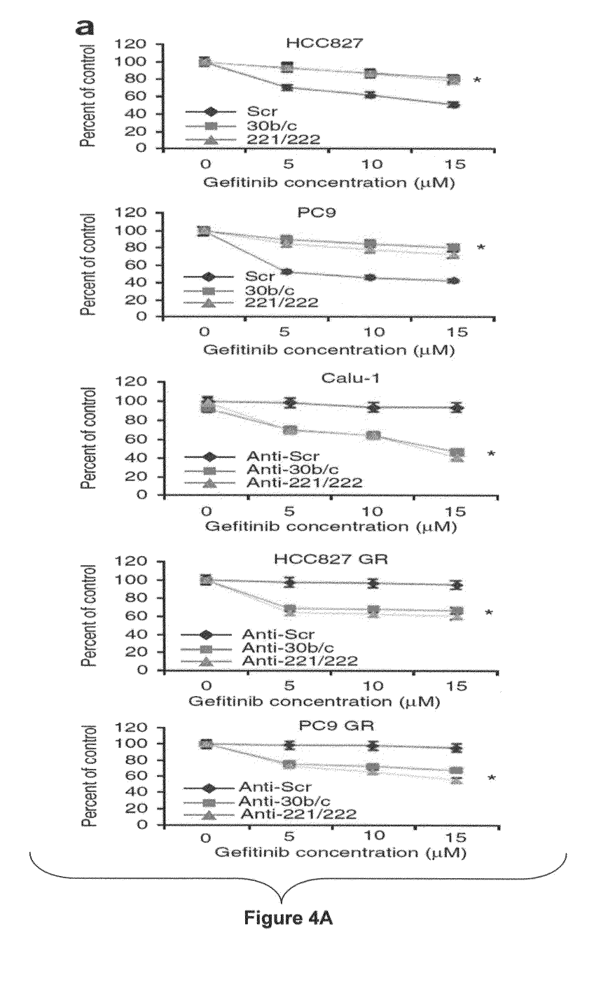

Having now found that EGFR regulates miR-221, miR-222, miR-30b and miR-30c, a role for these miRNAs in gefitinib-induced apoptosis in NSCLCs with wild-type EGFR (Calu-1 and A549 cells) compared to those with EGFR that has exon 19 deletions (PC9 and HCC827 cells) was determined Calu-1 and A549 cells were completely resistant to all concentrations of gefitinib tested (up to 20 μM); in contrast, the growth of PC9 and HCC827 EGFR mutant cells was significantly inhibited, even at low doses (0.1 μM) of gefitinib (FIG. 3A). Notably, after gefitinib treatment, there was marked miR-30b, miR-30c, miR-221 and miR-222 downregulation and increased amounts of BIM and APAF-1 protein, only in PC9 and HCC827 gefitinib-sensitive cells (FIGS. 3B, 3C). The concentration of phosphorylated ERKs was markedly lower in HCC827 and PC9 cells, but not in Calu-1 cells, compared to untreated cells (FIG. 3C).

To directly assess the relevance of miR-30b, miR-30c, miR-221 and miR-222 in gefitinib-induced apoptosis, the expression of these miRNAs in NSCLC cells was analyzed with acquired gefitinib resistance, obtained after long-term exposure to increasing drug concentrations: PC9 gefitinib-resistant (PC9 GR) cells with an EGFR Thr790 alteration and HCC827 gefitinib-resistant (HCC827 GR) cells with MET amplification. In contrast to the gefitinib-responsive parental cells, we did not observe lower expression of miR-30b, miR-30c, miR-221 and miR-222 or modulation of their relative targets after treatment with gefitinib (FIG. 3D and FIG. 12A).

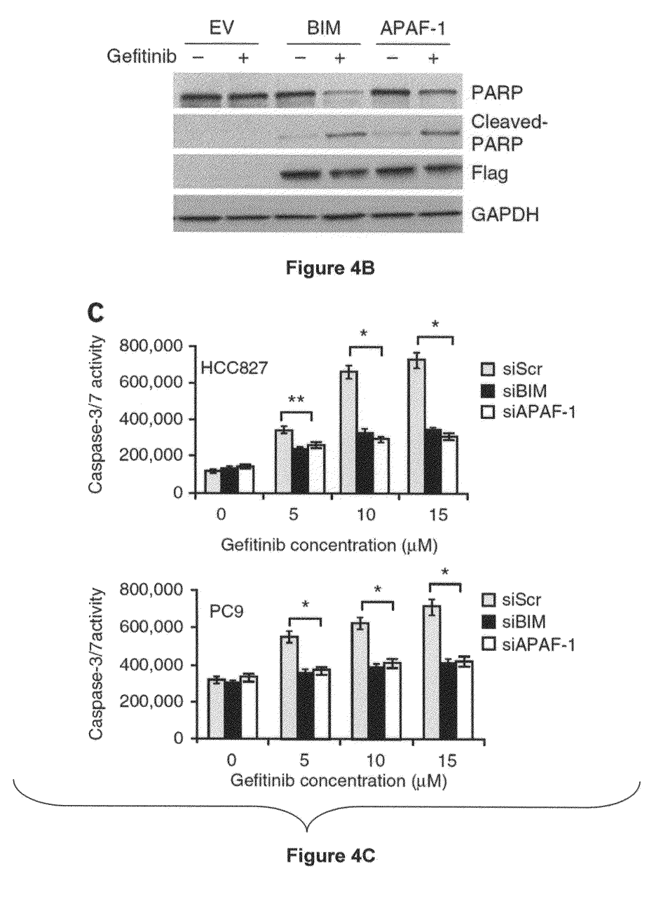

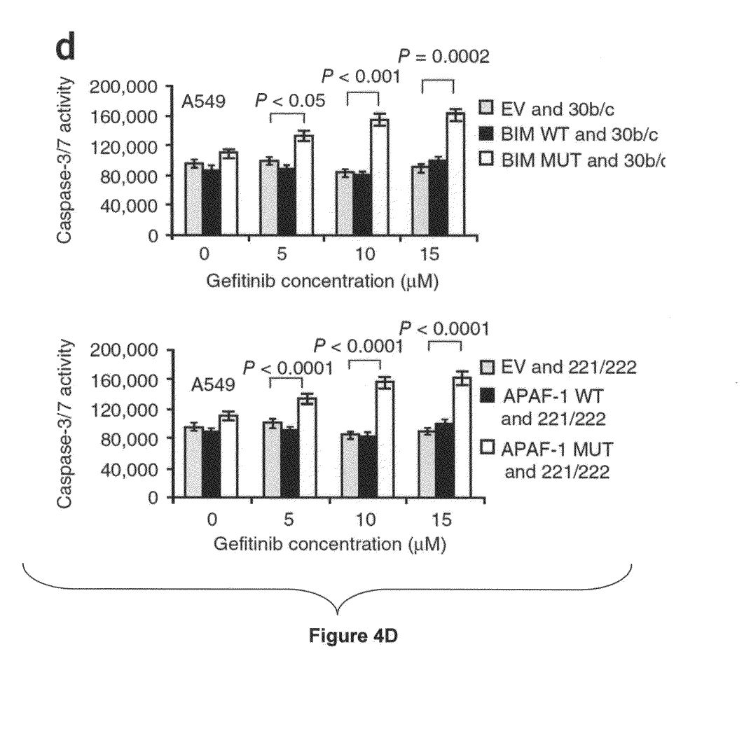

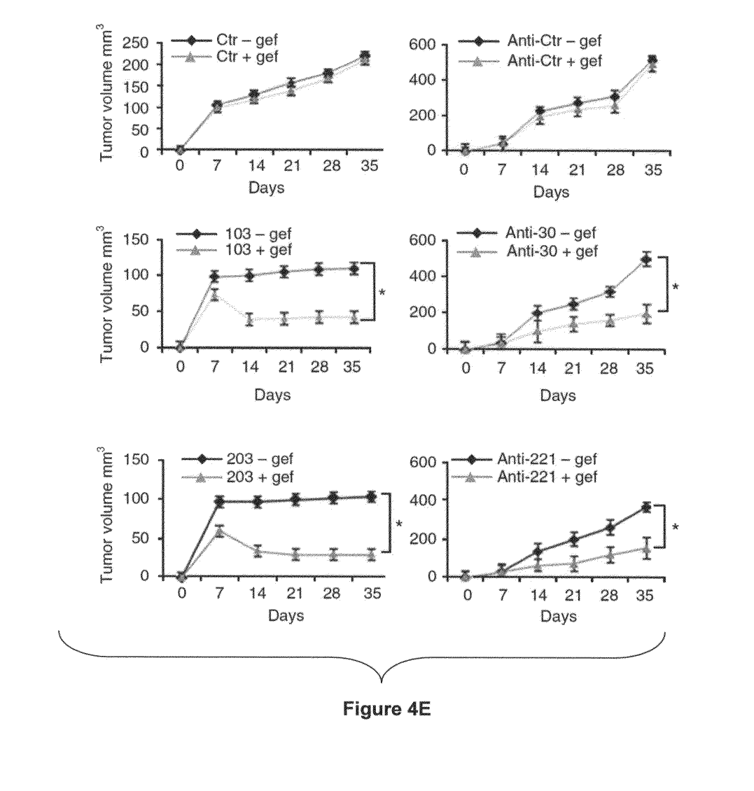

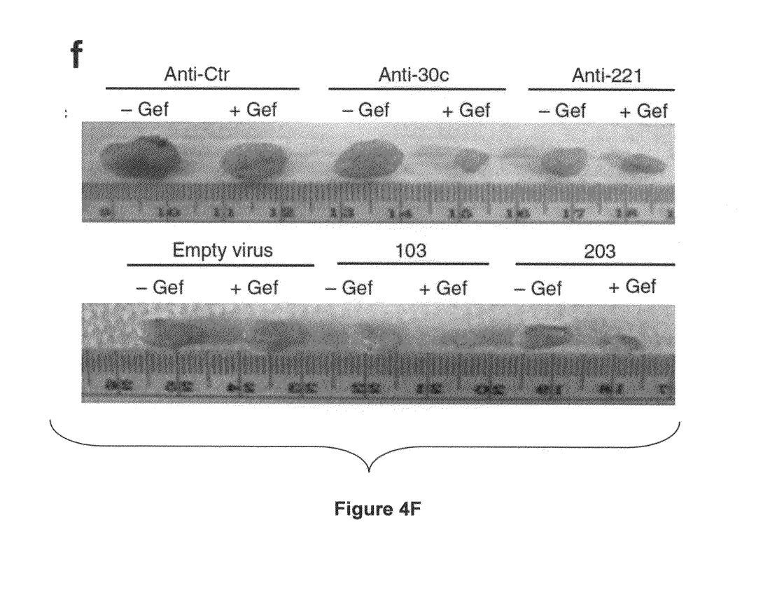

Of note, miR-30c, miR-221 and miR-222 overexpression in gefitinib-sensitive HCC827 and PC9 cells rendered these cells less responsive to treatment with gefitinib compared to parental PC9 and HCC827 cells (FIG. 4A and FIG. 13A), and knockdown of miR-30b, miR-30c, miR-221 and miR-222 led to increased gefitinib sensitivity in Calu-1, HCC827 GR and PC9 GR cells (FIG. 4A and FIG. 14B), showing that these miRNAs are key modulators of TKI resistance.

To investigate the contribution of APAF-1 and BIM downregulation mediated by miR-30b, miR-30c, miR-221 and miR-222 to the cellular TKI response, APAF-1 and BIM were overexpressed in A549 gefitinib-resistant cells. Gefitinib-induced poly-(ADP-ribose) polymerase (PARP) cleavage in cells was observed overexpressing BIM and APAF-1 but not in cells transfected with an empty vector plasmid (FIG. 4B). Conversely, the response to gefitinib was reduced by BIM and APAF-1 silencing in gefitinib-sensitive HCC827 and PC9 cells (FIG. 4C). Wild-type and mutated 3′ UTRs of BIM and APAF-1 (which we used for luciferase assays; FIG. 1E) downstream of BIM and APAF-1 coding sequences were cloned; and caspase-3/7 and viability assays were performed. There was no increase in cell death after treatment with gefitinib of A549 cells cotransfected mutations or deletions restored the apoptotic response to gefitinib, showing that the effects of both APAF-1 and BIM on gefitinib sensitivity were directly related to knockdown of these proteins mediated by miR-30b, miR-30c, miR-221 and miR-222 (FIG. 4D and FIG. 11C).

Because MET overexpression is associated with gefitinib resistance and because miR-30b, miR-30c, miR-221 and miR-222 are also regulated by MET, analytical results indicate that MET can mediate resistance to gefitinib treatment through the regulation of these miRNAs. Thus, the simultaneous inhibition of MET and EGFR can overcome gefitinib resistance in NSCLCs. Downregulation of miR-30b, miR-30c, miR-221 and miR-222 in Calu-1 and A549 cells overexpressing MET was observed after MET knockdown or treatment with the MET inhibitor SU11274 (FIGS. 14A, 14B).

In addition, there was increased caspase-3/7 activity and decreased cell viability in SU11274 treated Calu-1 and MET-KD Calu-1 cells that were exposed to different concentrations of gefitinib (FIGS. 14C, 14D). Taken together, these results show that MET overexpression induces resistance to gefitinib treatment in TKI-resistant Calu-1 cells through the upregulation of miR-30b, miR-30c, miR-221 and miR-222 and that inhibition of both EGFR and MET is needed to shut down these miRNAs and their survival effects.

Other miRNAs commonly deregulated by EGFR and MET, including miR-21, miR-29a, miR-29c and miR-100 (FIG. 1C), were downregulated in HCC827 and PC9 cells treated with gefitinib (FIG. 15A). Of note, downregulation of miR-21, miR-29a, miR-29c and miR-100 in HCC827 GR and PC9 GR cells after gefitinib treatment was not observed (FIG. 15B); however, enforced expression of miR-21, miR-29a, miR-29c and miR-100 increased gefitinib resistance in HCC827 and PC9 cells (FIG. 16A).

Thus, EGFR and MET control oncogenic signaling networks through common miRNAs. Whether miR-21 knockdown by oligonucleotide inhibitors of miRNAs could restore gefitinib sensitivity in NSCLC cells with de novo or acquired resistance was analyzed. miR-21 knockdown increased sensitivity to gefitinib-induced apoptosis in A549, HCC827GR and PC9GR cells, showing that this miRNA has a major role in the EGFR-MET signaling pathway (FIGS. 17A, 17B).

miR-103 and miR-203, which are strongly downregulated in MET-expressing Calu-1 cells, were also investigated (FIG. 1D). Treatment of Calu-1 cells with SU11274 increased (P<0.05) the expression of miR-103 and miR-203 (FIG. 18A).

Their targets, SRC and PKC-ε, exert pro-survival effects and contribute to gefitinib resistance by activating the AKT and ERK signaling pathways. Accordingly, overexpression of miR-103 and miR-203 in A549 cells was associated with reduced phosphorylation of AKT and its substrate glycogen synthase kinase 3 P (GSK3p) and reduced phosphorylation of the ERKs (FIG. 19A). MET induces gefitinib resistance through persistent PI3K-AKT and ERK signaling activation. These results show that MET overexpression controls gefitinib resistance through activation of the AKT-ERK pathways and is mediated, at least in part, by miR-103 and miR-203.

Enforced expression of miR-103 or miR-203 or silencing of PKC-ε and SRC increased the sensitivity of Calu-1 cells to gefitinib (as assessed by caspase-3/7 and viability assays; (FIGS. 19B, 19C). Notably, miR-103 and miR-203 expression decreased and SRC and PKC-ε expression consequently increased in HCC827 gefitinib-resistant cells with acquired MET amplification and gefitinib resistance compared to the HCC827 parental cells; thus showing that MET controls the response to TKIs, at least in part through miR-103, miR-203 and their respective targets (FIGS. 19D, 19E).

To analyze sensitivity to gefitinib in vivo, the inventors stably transfected A549 cells with GFP lentivirus constructs containing either full-length miR-103 or miR-203 or full-length inhibitors of miR-221 (anti-miR-221) and miR-30c (anti-miR-30c). Overexpression of miR-103 and miR-203 or knockdown of miR-221 and miR-30c resulted in marked inhibition of tumor growth and increased sensitivity to gefitinib-induced apoptosis in nude mice after 2 weeks of treatment (FIGS. 4E, 4F, and 20A). The downregulation of miR-221 and miR222 and the upregulation of miR-103 and miR-203 in the xenograft tumors by qRT-PCR (FIG. 20B).

MiR-103 and miR-203 Reduce NSCLC Cell Migration and Proliferation

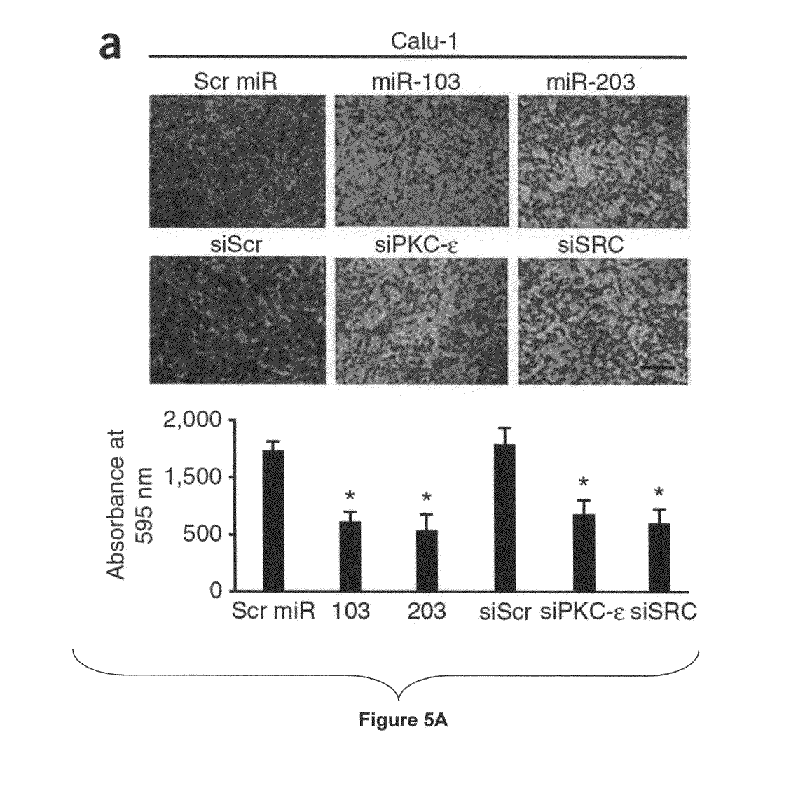

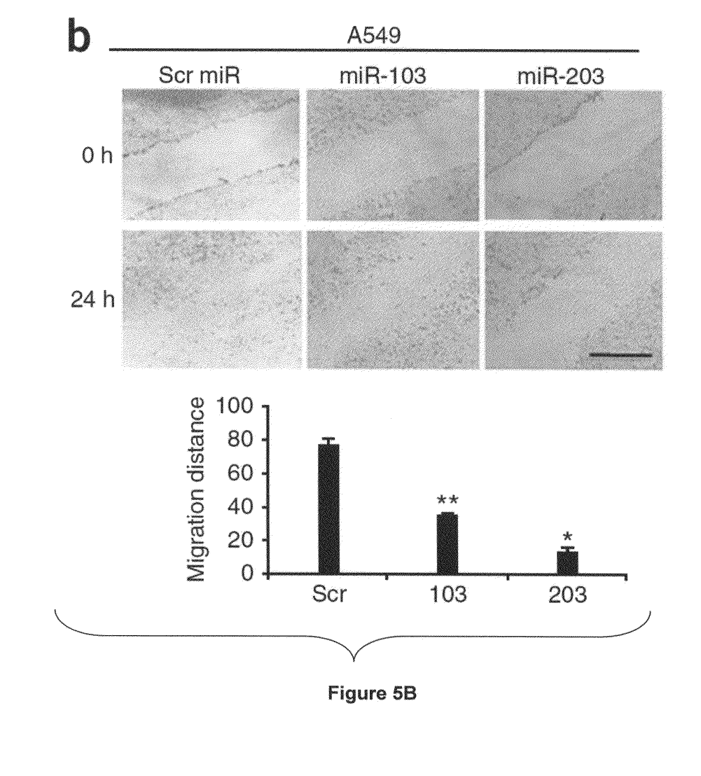

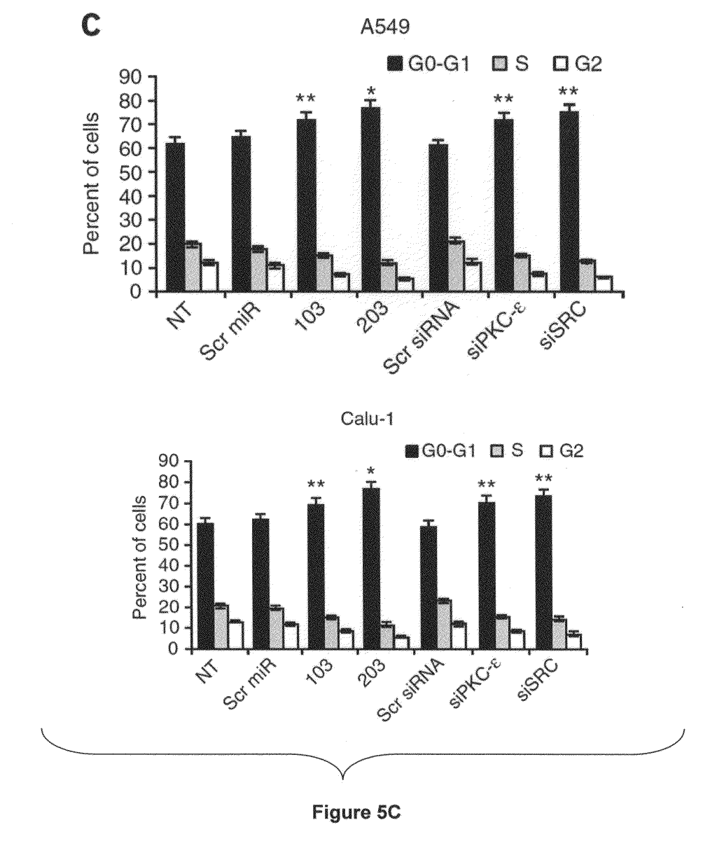

To further investigate the functional role of miR-103 and miR-203 in NSCLC tumorigenesis, the effects of miR-103 and miR-203 gain of function and the loss of PKC-ε and SRC on cell migration and cell cycle kinetics were assessed. Migration was reduced by about 60% compared to controls in cells with increased miR-103 and miR-203 expression or decreased PRKCE (PKC-ε) and SRC expression (FIG. 5A). These results were further confirmed using a wound-healing assay (FIG. 5B). In addition, A549 and Calu-1 cells transfected with miR-103, miR-203 or PRKCE (PKC-ε) and SRC siRNAs showed an increased G1 cell fraction and a corresponding decreased number of cells in the S and G2-M phases, with miR-203 and SRC siRNA having a slightly stronger effect as compared to miR-103 and PRKCE (PKC-ε) siRNA (FIG. 5C).

MiR-103 and miR-203 Promote the Mesenchymal-to-Epithelial Transition

There is an association between the epithelial-mesenchymal transition (EMT) and the development of chemoresistance, including resistance to EGFR-targeted therapy, that leads to recurrence of disease and metastasis. Although identifying the molecular events underlying EMT is an area under intense investigation, what triggers the onset of the EMT in tumor cells is unproven. It is now shown herein that there is a change in cellular shape of Calu-1 cells from a fibroblastoid morphology to an epithelial polarized phenotype after knockout of MET (FIG. 6A). It is now shown herein that this morphological change may be a result of a mesenchymal-to-epithelial transition. The expression of key EMT-associated markers was determined; and, in Calu-1 MET knockdown cells compared to Calu-1 Sh controls decreased expression of mesenchymal markers and increased E-cadherin expression (FIGS. 6B, 6C, 6D), strongly showing reversion of Calu-1 cells back to an epithelial phenotype after MET knockdown. Notably, in MET-KD cells, Snail protein expression was lower than in cells without MET knockdown, was localized to the cytoplasm (FIG. 6B), and the protein itself was presumably nonfunctional. There was no observed morphological change in EGFR-KD Calu-1 cells, in which miR-200c, miR-103 and miR-203 were not upregulated, as was the case in MET knockdown cells (FIG. 7A).

To determine whether miR-103 and miR-203 were involved in the mesenchymal-epithelial transition, these miRNAs were overexpressed in Calu-1 cells and observed downregulation of several mesenchymal markers and increased E-cadherin expression, indicating a role for these miRNAs in the mesenchymal-epithelial transition (FIGS. 6E, 6F, 6G and FIGS. 21A, 21B). In addition, silencing of PRKCE (PKC-ε) and SRC in Calu-1 cells increased the amount of E-cadherin and decreased the levels of SNAIL, ZEB1 (encoding zinc finger E-box binding 1), ZEB2 (encoding zinc finger E-box binding 2), vimentin and fibronectin mRNA compared to cells transfected with a siRNA control (FIG. 21C).

miR-103 targets Dicer; therefore, analytical investigation of the effects of Dicer knockdown on tumorigenesis and on gefitinib-induced apoptosis of NSCLCs was performed. Notably, near complete Dicer knockdown reduced not only gefitinib resistance but also the migration and expression of the mesenchymal markers of NSCLC cells, showing that miR-103 could also be involved in the mesenchymal-epithelial transition process through Dicer downregulation (Example 3 and FIG. 21).

By regulating the expression of specific miRNAs, MET orchestrates the convergence of several EMT-associated pathways, including the Dicer, SRC, PKC-ε and AKT pathways, supporting the possibility that MET targeting could be a strategy to control EMT and NSCLC progression.

Discussion of Example 1

EGFR and MET receptor tyrosine kinases, through regulation of expression of specific miRNAs, control the metastatic behavior and gefitinib resistance of NSCLCs.

MET is a regulator of miR-221 and miR-222 expression. To determine the pathway(s) involved in NSCLC tumorigenesis and drug resistance, we investigated miRNAs modulated by EGFR and MET tyrosine kinases. In particular, examined herein were miR-30b, miR-30c, miR-221 and miR-222, which are regulated by both EGFR and MET, and miR-103 and miR-203, which are regulated by MET only.

It is now shown herein that gefitinib treatment triggers programmed cell death through the downregulation of miR-30b, miR-30c, miR-221 and miR-222 and the consequent upregulation of APAF-1 and BIM in gefitinib-sensitive HCC827 and PC9 cells. Also, gefitinib treatment does not decrease miR-30b, miR-30c, miR-221 and miR-222 expression in gefitinib-resistant Calu-1, A549 and HCC827 GR cells as a result of MET overexpression. Therefore, EGFR inhibition alone in cells overexpressing MET is not sufficient to induce the downregulation of these miRNAs and, accordingly, cell death.

Also shown is that gefitinib resistance can be overcome by MET inhibitors, which downregulate miR-30b, miR-30c, miR-221 and miR-222 and sensitize NSCLCs to gefitinib or by anti-miR-221 anti-miR-222 and anti-miR-30c, which strongly increase gefitinib sensitivity in vitro and in xenograft mouse models in vivo. Taken together, these results show that the modulation of specific miRNAs, such as miR-30b, miR-30c, miR-221 and miR-222, have therapeutic applications to sensibilize lung tumors to TKI therapy.

PTEN loss, by partially uncoupling mutant EGFR from down-stream signaling and by activating EGFR, contributes to erlotinib resistance. PTEN is a miR-221 and miR-222 target. These two miRNAs have a role in the gefitinib resistance of NSCLC cells, not only through APAF-1 but also through PTEN regulation. Notably, overexpression of another miRNA targeting PTEN, miR-21, induced gefitinib resistance in HCC827 and PC9 gefitinib-sensitive cells.

Also shown herein is that miR-103 and miR-203, which are upregulated after MET silencing or treatment with the MET inhibitor SU11274, induce apoptosis in gefitinib-resistant NSCLCs, reduce mesenchymal markers and increase epithelial cell junction proteins compared to wild-type Calu-1 cells by downregulating the expression of PKC-ε, SRC and Dicer.

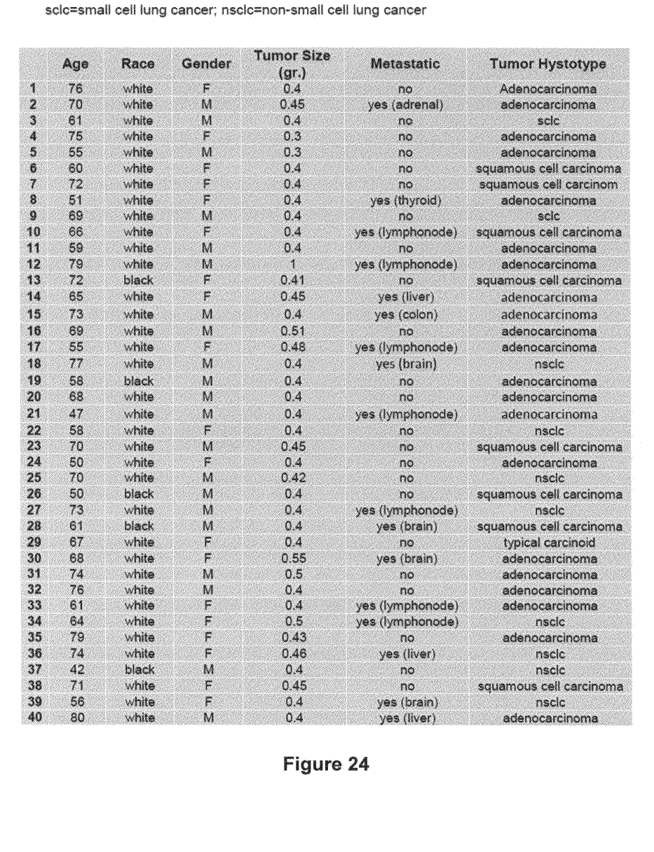

EMT as a role in acquired resistance to gefitinib in A549 cells, indicating that mesenchymal status is related to the ‘inherent resistance’ to gefitinib or erlotinib in NSCLCs. As shown in the model in FIG. 611, MET expression downregulates miR-103 and miR-203 and upregulates miR-221, miR-222, miR-30b and miR-30c, inducing gefitinib resistance, and epithelial-mesenchymal transition in NSCLCs. The identification of prognostic and predictive factors associated with sensitivity or resistance to anti-EGFR agents are important, and aberrant key signaling proteins, including RAS-MEK, AKT-mammalian target of rapamycin (mTOR) and MET kinase, are key targets.

As activation or amplification of MET signaling contributes to TKI resistance through multiple independent mechanisms and leads to the rapid evolution of drug resistance, stratifying NSCLCs based on MET expression or MET-regulated miRNAs, now allows for individualization of treatment. Such a stratification is useful to increase treatment efficacy by eliminating unnecessary side effects of a particular therapeutic regimen in NSCLC patients who would not benefit from that specific regimen.

In addition, the clinical validation studies on lung tumor specimens reveal that MET overexpression and the consequent absence of miR-103 and miR-203 are useful to identify primary lung tumors with metastatic capacity.

Further, reduced expression of miR-103 and miR-203 is predictive of more aggressive, early metastatic tumors.

Also, miRNAs combined with TKIs provides a new strategy to treat NSCLCs.

### Example 2

### TaqMan Array MicroRNA Cards

The TaqMan Array Human MicroRNA Card (Applied Biosystem) Set v3.0 is a two-card set containing a total of 384 TaqMan MicroRNA Assays per card that enables accurate quantification of 754 human miRNAs. Included on each array are three TaqMan MicroRNA Assays as endogenous controls to aid in data normalization and one TaqMan MicroRNA Assay not related to human as a negative control. An additional preamplification step was enabled by using Megaplex PreAmp Primers, Human Pool Set v3.0 for situations where sensitivity is of the utmost importance or where the sample is limiting.

In Vivo Experiments.

A549 cells were stably infected with a control miRNA, miR-103 and miR-203 or with control inhibitor of miRNA or a lentiviral inhibitor of miR-221 and miR-30c (SBI). We injected 5×10⁶ viable cells subcutaneously into the right flanks of 6-week-old male nude mice (Charles River Breeding Laboratories). Treatment was started 7 d after tumor cell inoculation. Gefitinib was administered Monday through Friday for 2 weeks as an oral gavage at concentrations of 200 mg per kg of body weight in 1% Tween 80 (Sigma) in sterile Milli-Q water (the vehicle control was 0.5% Tween 80 in sterile Milli-Q water). Tumor size was assessed twice per week using a digital caliper. Tumor volumes were determined by measuring the length (1) and the width (w) of the tumor and calculating the volume (V=lw²/2). We killed the mice 35 days after injection. Statistical significance between the control and treated mice was evaluated using a Student's t test. Mouse experiments were conducted after approval by the institutional animal care and use committee at Ohio State University.

Migration Assay.

Transwell insert chambers with an 8-μm porous membrane (Greiner Bio One) were used for the assay. Cells were washed three times with PBS and added to the top chamber in serum-free medium. The bottom chamber was filled with medium containing 10% FBS. Cells were incubated for 24 h at 37° C. in a 5% CO₂ humidified incubator. To quantify migrating cells, cells in the top chamber were removed by using a cotton-tipped swab, and the migrated cells were fixed in PBS, 25% glutaraldehyde and stained with crystal violet stain, visualized under a phase-contrast microscope and photographed. Crystalviolet-stained cells were then solubilized in acetic acid and methanol (1:1), and absorbance was measured at 595 nm.

Immunofluorescence.

Cells were grown on Lab-Tek II CC2 chamber slides (Nunc), fixed with 4% paraformaldehyde and permeabilized with 0.2% Triton X-100/PBS before blocking with 10% sheep serum (Caltag Laboratories). All the primary antibodies were from Abcam. Secondary antibodies were goat antibodies to mouse or rabbit coupled to Alexa 488 (Invitrogen). F-actin was stained by using a phalloidin reagent (Invitrogen). Cell nuclei were visualized with DAPI (Sigma). Slides were mounted with SlowFade Gold Antifade reagent (Invitrogen).

Cell Death and Cell Proliferation Quantification.

For detection of caspase 3/7 activity, cells were cultured in 96-well plates, in triplicate, treated with 5 μM, 10 μM or 15 μM gefitinig and analyzed using a Caspase-Glo 3/7 Assay kit (Promega) according to the manufacturer's instructions. Continuous variables are expressed as means±s.d. Cell viability was examined with 3-(4,5dimethylthiazol-2-yl)-2,5-dipheniltetrazolium bromide (MTS)-Cell Titer 96 AQueous One Solution Cell Proliferation Assay (Promega) according to the manufacturer's protocol. Metabolically active cells were detected by adding 20 μl of MTS to each well. After 1 h of incubation, the plates were analyzed in a Multilabel Counter (Bio-Rad Laboratories).

Statistical Analyses.

Student's t tests, one-way analysis of variance and Fisher's exact tests were used to determine statistical significance. A Pearson correlation coefficient was calculated to test the inverse relation between miR-103, miR-203, miR-221, miR-222, miR-30b and miR-30c and their putative targets and between MET and miR-103 and miR-203. Statistical significance for all the tests, assessed by calculating the P values, was defined as P<0.05.

### Example 3

### Depletion of Dicer by miR-103 Reduces Cell Migration and Promotes Gefitinib Sensitivity

Partial attenuation of Dicer by miR-103 fostered cell migration, while more complete Dicer knockdown impaired cell viability and reduced cell migration. There was a marked down-regulation of Dicer after MET silencing or miR-103 enforced expression (FIG. 22A), showing that the almost complete silencing of Dicer by miR-103 in this system can promote the reduction of cancer cell motility and induce programmed cell death. To address this experimentally, we transfected A549 and Calu-1 cells with Dicer siRNA, inducing a significant knockdown of Dicer (FIG. 22B) to levels similar to those achieved by miR-103 expression. Global attenuation of Dicer in A549 and Calu-1 cells had a significant effect on both cell migration and gefitinib resistance as compared to control cells (FIGS. 22C, 22D). Moreover, Dicer silencing reduced the expression of mesenchymal markers in Calu-1 cells and increased E-cadherin expression levels, showing that miR-103 induces mesenchymalepithelial transition not only through PKC-ε but also through Dicer downregulation (FIG. 22E).

Luciferase Assay

The 3′ UTRs of human APAF-1, BIM (BCL2L11, PKC-ε and SRC genes were PCT amplified using the following primers (SEQ ID NOS 1-12, respectively, in order of appearance):

APAF-1 Fw
5′ TCT AGA CTA ATG AAA CCC TGA TAT CAA C 3′
APAF-1 Rw
5′ TCT AGA ACTGCTACCCTGAGGCACAGCCT 3′
BIM FW:
5′ TCTAGACTGGATGGGACTACCTTTCTGTTC 3′
BIM RW:
5′ TCTAGACATAATCCTCTGAGAATAGGCCG 3'
PKC-ε FW D
5′ TCTAGAGTGACATGCAATGGCAACTCATGTGGAC 3′
PKC-ε RW D
5′ TCTAGAACAAAGAATCCCCAACACACCCCCCCAT 3′
PKC-ε FW S
5′ TCTAGATGATGCCCTGAGAGCCCACTGCAGTT 3′
PKC-ε RW S
5′ TCTAGATTGCTTCACTGCCAGGAGCCCCTGA 3′
SRC-1-21Fw
5′- GCT CTA GAG CGC AGC ACA AGG CCT TGC CTG
GCC TGA TGA T -3′
SRC-1-2RW:
5′- GCT CTA GAG CCA TGG CAG TGG GTA ACA CGT
CCT CTT TCA C -3′
SRC-3-4FW
5′- GCTCTAGATCCCTGTGTGTGTGTATGTGTGTGCATGTGTGCGT 3′
SRC-3-4Rw
5′- GCT CTA GAG CGG AGA GGG ATT TGA GAG CTC
GCT GGG GTG A -3′

and cloned downstream of the Renilla luciferase stop codon in pGL3 control vector (Promega). These constructs were used to generate, by inverse PCR, the p3″-UTRs-mutant-plasmids using the following primers (SEQ ID NOS 13-24, respectively, in order of appearance):

APAF-1 Mut FW
5′ GTGGTTGGATGAATAATATTAATCTCCTTTTTCCC 3′
APAF-1 Mut Rw
5′ GGGAAAAAGGAGATTAATATTATTCATCCAACCAC 3′
BIM MUT FW:
5′ GTGTAAGAATGGTGCAGTGTGTTTTCCCCCTC 3′
BIM MUT RW
5′ GAGGGGGAAAACACACTGCACCATTCTTACAC 3′
PKC-ε FW MUT 1
5′ GAGA TTTTTGTATA TAGTGTTAGGCCT GTGGAATTAA TTCG 3′
PKC-ε RW MUT 1
5′ CGAATTAATTCCACAGGCCTAACACTATATACAAAAATCTC 3′
PKC-ε FW MUT 2
5′ CGTTGCATATAGAGGTATCAATGTTCAGGCATATTATAAAAC 3′
PKC-ε RW MUT 2
5′ GTTTTATAATATGCCTGAACATTGATACCTCTATATGCAACG 3′
SRC-3° Mut Fw
5′ CCAAACATGTTGTACCATGGCCCCCTCATCATAG 3′
SRC-3° Mut Rw
5′ CTATGATGAGGGGGCCATGGTACAACATGTTTGG 3′
SRC-4° mut Fw
5′ GGCCAAGCAGTGCCTGCCTATGAACTTTTCCTTTCATACG 3′
SRC-4° mut RW
5′ CGTATGAAAGGAAAAGTTCATAGGCAGGCACTGCTTGGCC 3′

MeG01 cells were cotransfected with 1 μg of p3′UTR-APAF-1, p3′UTR-BIM, p3′UTR-PKCε, p3′UTR-SRC and with p3′UTRmut-APAF-1, p3′UTRmut-BIM, p3′UTRmut-PKCε, p3′UTRmut-SRC plasmids and 1 μg of a Renilla luciferase expression construct pRL-TK (Promega) by using Lipofectamine 2000 (Invitrogen). Cells were harvested 24 h post-transfection and assayed with Dual Luciferase Assay (Promega) according to the manufacturer's instructions. Three independent experiments were performed in triplicate.

Western Blot Analysis

Total proteins from NSCLC were extracted with radioimmunoprecipitation assay (PIRA) buffer (0.15 mM NaCl, 0.05 mM Tris-HCl, pH 7.5, 1% Triton, 0.1% SDS, 0.1% sodium deoxycholate and 1% Nonidet P40). Sample extract (50 μg) was resolved on 7.5-12% SDS-polyacriylamide gels (PAGS) using a mini-gel apparatus (Bio-Rad Laboratories) and transferred to Hybond-C extra nitrocellulose. Membranes were blocked for 1 h with 5% nonfat dry milk in Tris-buffered saline containing 0.05% Tween 20, incubated overnight with primary antibody, washed and incubated with secondary antibody, and visualized by chemiluminescence.

The following primary antibodies were used: Apaf-1, Snail, Slug (abcam), Src, Met, Dicer, Vimintin, E-cadherin, Zeb1, Zeb-2 (Santa Cruz), Bim, pErks, total Erks, pAkt, total Akt, GAPDH, Parp (cell signaling, Pkc-ε, (BD transduction lab), β-actin antibody, Fibronectin (Sigma). A secondary anti-rabbit or anti-mouse immunoglobulin G (IgG) antibody peroxidase conjugate (Chemicon) was used.

Real-Time PCR

Real-time PCR was performed using a standard TaqMan PCR Kit protocol on an Applied Biosystems 7900HT Sequence Detection System (Applied Biosystems). The 10 pl PCR reaction included 0.67 pl RT product, 1 pl TaqMan Universal PCR Master Mix (Applied Biosystems), 0.2 mM TaqMan probe, 1.5 mM forward primer and 0.7 mM reverse primer. The reactions were incubated in a 96-well plate at 95° C. for 10 mM, followed by 40 cycles of 95° C. for 15 s and 60° C. for 1 mM All reactions were run in triplicate. The threshold cycle (CT) is defined as the fractional cycle number at which the fluorescence passes the fixed threshold. The comparative CT method for relative quantization of gene expression (Applied Biosystems) was used to determine miRNA and genes expression levels. The y axis represents the 2^{(-ΔCT)}, or the relative expression of the different miRs and genes. MiRs expression was calculated relative to U44 and U48 rRNA (for microRNAs) and to GAPDH (for genes). Experiments were carried out in triplicate for each data point, and data analysis was performed by using software (Bio-Rad).

shRNA Lentiviral Particles Transduction

Cells were plated in a 12-well plate 24 hours prior to viral infection and incubated overnight with 1 ml of complete optimal medium (with serum and antibiotics). The day after the medium was removed and 1 ml of complete medium with Polybrene (5 μg/ml) was added. The day after, cells were infected by adding 50 μl of control shRNA, shEGFR, shMET Lentiviral Particles (Santa Cruz) to the cultures. Stable clones were selected via 1 pg/ml of Puromycin dihydrochloride.

RNA Extraction and Northern Blotting

Total RNA 1 5 was Zextracted with TRIzol ZLsolution (Invitrogen), according to the manufacturer's instructions and the integrity of RNA was assessed with an Agilent BioAnalizer 2100 (Agilent, Palo Alto, Calif., USA). Northern blotting was performed. The oligonucleotides used as probes (SEQ ID NOS 25-30, respectively, in order of appearance) were the complementary sequences of the mature miRNA (miRNA registry):

miR-103:
5′TCATAGCCCTGTACAATGCTGCT3′;
miR-203:
5′CTAGTGGTCCTAAACATTTCAC3′;
miR30b:
5′AGCTGAGTGTAGGATGTTTACA
miR30c:
5′GCTGAGAGTGTAGGATGTTTACA3′;
miR-221:
5′GAAACCCAGCAGACAATGTAGCT3′;
miR-221:
5′ACCCAGTAGCCAGATGTAGTAGCT3′

PKCε, SRC, BIM, APAF-1 siRNAs Transfection.

Cells were cultured to 50% confluence and transiently transfected using Lipofectamine 2000 with 100 nM anti-PKC-H, anti-SRC, anti-BIM anti-APAF-1 or control siRNAs (Santa Cruz), a pool of three target specific 20-25 nt siRNAs designed to knock down gene expression.

MiRNA Locked Nucleic Acid In Situ Hybridization of Formalin Fixed, Paraffin-Embedded Tissue Section.

In situ hybridization (ISH) was carried out on deparaffinized human lung tissues using a protocol, which includes a digestion in pepsin (1.3 mg/ml) for 30 minutes. The sequences of the probes containing the dispersed locked nucleic acid (LNA) modified bases with digoxigenin conjugated to the 5′ end were:

miR-222
(SEQ ID NO: 31)
(5′) ACCCAGTAGCCAGATGTAGCT;
miR103-
(SEQ ID NO: 32)
(5′) AGCAGCATTGTACAGGGCTATGA (3′);
miR-203-
(SEQ ID NO: 33)
(5′) CTAGTGGTCCTAAACATTTCAC

The probe cocktail and tissue miRNA were co-denatured at 60° C. for 5 minutes, followed by hybridization at 37° C. overnight and stringency wash in 0.2×SSC and 2% bovine serum albumin at 4° C. for 10 minutes. The probe-target complex was seen due to the action of alkaline phosphatase on the chromogen nitroblue tetrazolium and bromochloroindolyl phosphate (NBT/BCIP). Negative controls included the use of a probe which should yield a negative result in such tissues (scrambled miRNA). No counterstain was used, to facilitate co-labeling for PKC-ε, APAF-1, SRC, BIM and MET proteins.

After in situ hybridization for the miRNAs, the slides were analyzed for immunohistochemistry (IHC) using the optimal conditions for SRC (1:100, cell conditioning for 30 minutes), PKC-ε (1:10, protease digestion for 4 minutes) BIM (1:100, cell conditioning for 30 minutes), APAF-1 (1:25, cell conditioning for 30 minutes) and MET (1:50, cell conditioning for 30 minutes). The 30 independent tumor specimens were analyzed by IHC using the optimal condition for MET (1:50, cell conditioning for 30 minutes) and EGFR (1:100, cell conditioning for 30 minutes).

For the immunohistochemistry, the Ultrasensitive Universal Fast Red or DAB systems from Ventana Medical Systems was used. The percentage of tumor cells expressing PKC-ε, SRC, BIM, APAF-1, MET and miR-103, miR-203, miR-30c, miR-221/miR-222 was then analyzed with emphasis on co-localization of the respective targets. Co-expression analysis was done with the Nuance system (Cambridge Research Institute) per the manufacturer's recommendations.

Lung Cancer Samples and Cell Lines

110 cancer lung tissues were purchased from US Biomax, Inc. 40 lung tumor tissue samples were provided from the Department of Pathology, Ohio State University. All human tissues were obtained according to a protocol approved by the Ohio State Institutional Review Board. Human Calu-1 cell lines were grown in Dulbecco's modified Eagle's medium containing 10% heat-inactivated fetal bovine serum (FBS) and with 2 mM L-glutamine and 100 Uml-1 penicillin-streptomycin. A549, H460, H1299, H1573, H292, HCC827, PC9, HCC827 GR, PC9GR cell lines were grown in RPMI containing 10% heat-inactivated FBS and with 2 mM L-glutamine and 100 Uml-1 penicillin-streptomycin.

Bioinformatics Analysis

Bioinformatics analysis was performed by using these specific programs: Targetscan, Pictar, RNhybrid.

Generation of Stable Clones with miR-103 and miR-203 Overexpression and miR-221, miR-30c Downregulation

A549 cells were stably infected with the Human pre-microRNA Expression Construct Lenti-miR expression plasmid containing the full-length miR-103, miR-203 or the anti-miR-221, miR-30c and the GFP gene under the control of two different promoters (System Biosciences). An empty vector was used as control. Pre-miRs expression and control constructs were packaged with pPACKH1 Lentivector Packaging Plasmid mix (System Biosciences) in a 293TN packaging cell line. Viruses were concentrated using PEGit Virus Precipitation Solution, and titers were analyzed using the UltraRapid Lentiviral Titer Kit (System Biosciences). Infected cells were selected by FACS analysis (FACScalibur; BD Bioscience). Infection efficiency >90% was verified by fluorescent microscopy and confirmed by real-time PCR for miRs expression.

Generation of miR-30b/c- and 221/222-Insensitive BIM and APAF-1 cDNAs

Bim and APAF-1 WT and mutated 3′UTRs were amplified and cloned downstream of the APAF-1 and BIM coding sequences (Origene) by using the following primers (SEQ ID NOS 34-37, respectively, in order of appearance):

APAF-1 FW
5′ GGCCGGCC CTA ATG AAA CCC TGA TAT CAA C 3′
APAF-1 RW
5′ GGCCGGCC ACTGCTACCCTGAGGCACAGCCT 3′
BIM FW:
5′GGCCGGCCCTGGATGGGACTACCTTTCTGTTC 3′
BIM RW:
5′GGCCGGCGCATAATCCTCTGAGAATAGGCCC 3′

The constructs were then used to perform viability and caspase 3/7 assays. Experiments were performed at least three times in triplicate.

Scratch Assay

A549 cells were transfected with control miR, miR-103 or miR-203 for 72 h. 24 h after transfection cells were incubated with medium 5% FBS. Images were acquired directly after scratching (0 h) and after 24 h. Quantization of migration distance using Image J software. The distance covered was calculated by converting pixel to millimeters.

Cell-Cycle Analysis

For cell-cycle analysis, cells were plated in 6 cm dishes, transfected as indicated in the figures, trypsinized, washed in PBS, and fixed with ice-cold 70% ethanol while vortexing. Cells were rehydrated in PBS and stained 30 min at RT with propidium iodide (50 mg/ml PI, 0.5 mg/ml RNase in PBS) prior to flow-cytometric analysis. Every experiment was repeated 5 times independently, with two replicas for each sample.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

1. A composition comprising a nucleic acid selected from the group consisting of: isolated nucleotides 154 through 160 of the miR-221/222 binding site of APAF-1 (5′-ATGTAGC-3′); isolated nucleotides 288 through 294 of the miR-30b binding site of BIM (5′-TGTTTACA-3′); isolated nucleotides complementary to the 27 through 33 of the miR-103 binding site of PKC-ε (complement: 3′-ACGACG-5′); isolated nucleotides complementary to nucleotides 1517 through 1523 of the miR-103 binding site of PKC-ε (complement: 3′-ACGACG); isolated nucleotides complementary to nucleotides 1564 through 1570 of the miR-103 binding site of PKC-ε (complement: 3′-ACGACG-5′); isolated nucleotides complementary to nucleotides 656 through 662 of the miR-203 binding site of SRC (complement: 3′-UAAAGU-5′); isolated nucleotides complementary to nucleotides 1116 through 1122 of the miR-203 binding site of SRC (complement: 3′-UAAAGU-5′); isolated nucleotides complementary to nucleotides 1595 through 1601 of the miR-203 binding site of SRC (complement: 3′-UAAAGU-5′); and isolated nucleotides complementary to nucleotides 1706 through 1712 of the miR-203 binding site of SRC (complement: 3′-UAAAGU-5′).

2. An isolated nucleic acid comprising a nucleic acid selected from the group consisting of: 5′-ATGTAGC-3′; 5′-TGTTTACA-3′; 3′-ACGACG-5′; and 3′-UAAAGU-5′.

3. The isolated nucleic acid of claim 2, which further comprises an element selected from the group consisting of: promoter;
enhancer; repeat; marker; and reporter.

4. The isolated nucleic acid of claim 2, which is a probe, primer, miRNA, plasmid, vector, virus, cell, or modified organism.

5. A composition of matter comprising at least one miR selected from the group consisting of: miR-103; miR-203; anti-miR-30; and anti-miR-221.

6. The composition of claim 5, which comprises at least two miRNAs selected from the group consisting of: miR-103; miR-203; anti-miR-30; and anti-miR-221.

7. The composition of claim 5, which comprises at least three miRNAs selected from the group consisting of: miR-103; miR-203; anti-miR-30; and anti-miR-221.

8. The composition of claim 5, which comprises miR-103; miR-203; anti-miR-30; and anti-miR-221.

9. The composition of claim 1, which further comprises a chemotherapeutic treatment.

10. The composition of claim 1, which further comprises a lung cancer chemotherapeutic treatment.

11. The composition of claim 1, which further comprises an epidermal growth factor receptor (EGFR) inhibitor.

12. The composition of claim 1, which further comprises a tyrosine kinase inhibitor (TKI).

13. The composition of claim 1, which further comprises a monoclonal antibody selected from the group consisting of: cetuximab; panitumumab; zalutumumab; nimotuzmab; and matuzumab.

14. The composition of claim 1, which further comprises a small molecule selected from the group consisting of: gefitinib; erlotinib; lapatinib; AP26113; and potato carboxypeptidase inhibitor.

15. The composition of claim 1, which further comprises getifitinib.

16. The composition of claim 1, which further comprises a PKC-ε expression agonist.

17. The composition of claim 1, which further comprises a MET inhibitor.

18. The composition of claim 1, which further comprises SU11274.

19. The composition of claim 1, which further comprises a DICER inhibitor.

20. The composition of claim 1, which further comprises a E-cadherin expression agonist.

21. The composition of claim 1, which further comprises an adjuvant, excipient, or other pharmaceutically-acceptable compositions.

22. The composition of claim 1, formulated for injection, transfusion, ingestion, or transmembrane conveyance.

23. A method to downregulate DICER in a mammalian cell, comprising increasing miR-103 and/or miR-203 availability in a mammalian cell, and downregulating DICER in the mammalian cell.

24. A method to decrease migration in a mammalian cancer cell, comprising increasing miR-103 and/or miR-203 availability in a mammalian cancer cell, and decreasing migration of the mammalian cancer cell.

25. A method to decrease EGFR chemotherapy resistance of a mammalian cancer cell, comprising increasing miR-103 and/or miR-203 availability in a mammalian cancer cell, and decreasing EGFR chemotherapy resistance of the mammalian cancer cell.

26. A method to decrease gefitinib resistance of, or increase gefitinib sensitivity in, a mammalian cancer cell, comprising increasing miR-103 and/or miR-203 availability in a mammalian cancer cell, and decreasing gefitinib resistance of, or increasing gefitinib sensitivity in, the mammalian cancer cell.

27. A method to decrease expression of mesenchymal markers in a mammalian cancer cell, comprising increasing miR-103 and/or miR-203 availability in a mammalian cancer cell, and decreasing expression of mesenchymal markers in the mammalian cancer cell.

28. A method to increase expression of E-cadherin expression in a mammalian cancer cell, comprising increasing miR-103 and/or miR-203 availability in a mammalian cancer cell, and increasing expression of E-cadherin expression in the mammalian cancer cell.

29. A method to induce mesenchymal-epithelial transition in a mammalian cancer cell, comprising increasing miR-103 and/or miR-203 availability in a mammalian cancer cell, and inducing mesenchymal-epithelial transition in the mammalian cancer cell.

30. The method of claim 29, wherein the mesenchymal-epithelial transition is induced through PKC-ε and/or DICER.

31. A method to induce programmed cell death in a mammalian cancer cell, comprising increasing miR-103 and/or miR-203 availability in a mammalian cancer cell, and inducing programmed cell death of the mammalian cancer cell.

32. A method to downregulate AKT/ERK in a mammalian cancer cell, comprising increasing miR-103 and/or miR-203 availability in a mammalian cancer cell, and downregulating AKT/ERK in the mammalian cancer cell.

33. (canceled)

34. The method of claim 26, wherein the cancer cell is a lung cancer cell.

35. The method of claim 26, wherein the cancer cell is a non-small cell lung adenocarcinoma cell.

36. The method of claim 26, wherein the cancer cell is an epidermal carcinoma cell.

37. A method to inhibit tumor growth in a mammal in need of tumor growth inhibition, comprising administering a tumor growth-inhibiting amount of at least one nucleic acid selected from the group consisting of: isolated nucleotides 154 through 160 of the miR-221/222 binding site of APAF-1 (5′-ATGTAGC-3′); isolated nucleotides 288 through 294 of the miR-30b binding site of BIM (5′-TGTTTACA-3′); isolated nucleotides complementary to nucleotides 27 through 33 of the miR-103 binding site of PKC-ε (complement: 3′-ACGACG-5′); isolated nucleotides complementary to nucleotides 1517 through 1523 of the miR-103 binding site of PKC-ε (complement: 3′-ACGACG); isolated nucleotides complementary to nucleotides 1564 through 1570 of the miR-103 binding site of PKC-ε (complement: 3′-ACGACG-5′); isolated nucleotides complementary to nucleotides 656 through 662 of the miR-203 binding site of SRC (complement: 3′-UAAAGU-5′); isolated nucleotides complementary to nucleotides 1116 through 1122 of the miR-203 binding site of SRC (complement: 3′-UAAAGU-5′); isolated nucleotides complementary to nucleotides 1595 through 1601 of the miR-203 binding site of SRC (complement: 3′-UAAAGU-5′); and isolated nucleotides complementary to nucleotides 1706 through 1712 of the miR-203 binding site of SRC (complement: 3′-UAAAGU-5′).

38. A method to inhibit tumor growth in a mammal in need of tumor growth inhibition, comprising administering a tumor growth-inhibiting amount of a nucleic acid selected from the group consisting of: 5′-ATGTAGC-3′; 5′-TGTTTACA-3′; 3′-ACGACG-5′; and 3′-UAAAGU-5′.

39. A method to inhibit tumor growth in a mammal in need of tumor growth inhibition, comprising administering a tumor growth-inhibiting amount of at least one miR selected from the group consisting of: miR-103; miR-203; anti-miR-30; and anti-miR-221.

40-42. (canceled)

43. The method of claim 37, which further comprises administering a chemotherapeutic treatment.

44. The method of claim 37, which further comprises administering lung cancer chemotherapeutic treatment.

45. The method of claim 37, which further comprises administering an epidermal growth factor receptor (EGFR) inhibitor.

46. The method of claim 37, which further comprises administering a tyrosine kinase inhibitor (TKI).

47. The method of claim 37, which further comprises administering a monoclonal antibody selected from the group consisting of: cetuximab; panitumumab; zalutumumab; nimotuzmab; and matuzumab.

48. The method of claim 37, which further comprises administering a small molecule selected from the group consisting of: gefitinib; erlotinib; lapatinib; AP26113; and potato carboxypeptidase inhibitor.

49. The method of claim 37, which further comprises administering gefitinib.

50. The method of claim 37, which further comprises administering a PKC-ε expression agonist.

51. The method of claim 37, which further comprises administering a MET inhibitor.

52. The method of claim 37, which further comprises administering SU11274.

53. The method of claim 37, which further comprises administering a DICER inhibitor.

54. The method of claim 37, which further comprises administering an E-cadherin expression agonist.

55. The method of claim 37, which further comprises administering an adjuvant, excipient, or other pharmaceutically-acceptable compositions.

56. The method of claim 37, wherein administration is via injection, transfusion, ingestion, or transmembrane conveyance.

57. The method of claim 37, wherein the tumor is a lung tumor.

58. The method of claim 37, wherein the tumor is a lung carcinoma.

59. The method of claim 37, wherein the tumor is a lung adenocarcinoma.

60. The method of claim 37, wherein the tumor is non-small cell lung carcinoma.

61. The method of claim 37, wherein the tumor growth is reduced by at least 10%, at least 20% at least 30%, at least 40%, at least 50%, or at least 60% compared to control.

62. A method to promote wound healing in a mammal in need of wound healing promotion, comprising administering a wound healing-promoting amount of the composition of claim 1 and promoting wound healing the mammal.

63. A kit comprising the composition of claim 1.

64. A cell comprising the composition of claim 1.

65. A mouse comprising the composition of claim 1.
